(19) United States
(12) Patent Application Publication
(10) Pub. No.: US 2017/0218456 A1
(43) Pub. Date: Aug. 3, 2017

(54) Systems, Devices and Methods for Constructing and Using a Biomarker

(71) Applicant: ONTARIO INSTITUTE FOR CANCER RESEARCH, Toronto (CA)

(72) Inventors: John Bartlett, Toronto (CA); Paul Boutros, Toronto (CA); Victoria Sabine, Puslinch (CA); Syed Haider, West Midlands (GB); Maud H.W. Starmans, Kerkrade (NL); Cindy Qianli Yao, Toronto (CA); Jianxin Wang, Fort Erie (CA)

(21) Appl. No.: 15/328,108

(22) PCT Filed: Jul. 23, 2015
(86) PCT No.: PCT/CA2015/050692

§ 371 Date: Jan. 23, 2017

Related U.S. Application Data
(60) Provisional application No. 62/027,966, filed on Jul. 23, 2014.
(60) Provisional application No. 62/085,416, filed on Nov. 28, 2014.

(51) Int. Cl. C12Q 1/68; G06F 19/00; A61K 31/138; G06F 19/20; A61K 31/5685

(52) U.S. Cl. CPC C12Q 1/6886; G06F 19/20; A61K 31/5685; A61K 31/138; G06F 19/3431; C12Q 2600/158; C12Q 2600/118; C12Q 2600/166; C12Q 2600/106

(57) ABSTRACT

Methods, systems, devices and computer implemented methods of prognosing or classifying patients using a biomarker comprising a plurality of subnetwork modules are disclosed. In some embodiments, the method comprises determining an activity of a plurality of genes in a test sample of a patient, wherein the plurality of genes are associated with the plurality of subnetwork modules. An expression profile is constructed using the activity of the plurality of genes. The dysregulation of each of the plurality of subnetwork modules is determined by calculating a score proportional to a degree of dysregulation in each of the plurality of subnetwork modules from the expression profile. The patient is prognosed or classified by inputting each dysregulation score into a model for predicting patient outcomes for patients having a disease, and inputting a clinical indicator of the patient into the model, to obtain a risk associated with the disease.

### TECHNICAL FIELD

This disclosure relates generally to biomarkers, and more particularly to systems, devices, and methods for constructing and using biomarkers.

### BACKGROUND

The treatment of early luminal (estrogen receptor positive) breast cancer is both a major success story and an ongoing clinical challenge. Targeted anti-endocrine therapies have significantly reduced mortality over the last 30-40 years [1,2], but luminal disease still leads to the majority of deaths from early breast cancer. To address this urgent clinical need, research has focused on improving anti-endocrine therapies (e.g. third-generation aromatase inhibitors) [2] and on generating a plethora of “prognostic markers” to personalize risk stratification for luminal breast cancer patients [3]. These strategies have led to a statistically significant, but clinically modest, improvement in outcome [2,3].

More broadly, human disease is complex, caused by the interaction of genetic, epigenetic and environmental insults. These interactions allow a specific disease phenotype to arise in many different ways, with a far greater diversity of molecular underpinnings than phenotypic consequences. Molecular heterogeneity within a disease is believed to underlie poor clinical trial results for some therapies [43] and the poor performance of many genome-wide association studies [44-46].

A new solution is thus needed for overcoming the shortfalls of the solutions currently available in the market in respect of not just early luminal (estrogen receptor positive) breast cancer, but also a wider range of diseases and other phenotypes.

### SUMMARY

In an aspect, there is disclosed a method of prognosing or classifying a patient using a biomarker comprising a plurality of subnetwork modules, said method comprising: determining an activity of a plurality of genes in a test sample of the patient, said plurality of genes associated with the plurality of subnetwork modules; constructing an expression profile using the activity of the plurality of genes; determining dysregulation of each of the plurality of subnetwork modules by calculating a score proportional to a degree of dysregulation in each of the plurality of subnetwork modules from said expression profile; prognosing or classifying the patient by: inputting each dysregulation score into a model for predicting patient outcomes for patients having a disease, the model trained with a plurality of reference dysregulation scores and a plurality of reference clinical indicators; and inputting a clinical indicator of the patient into the model to obtain a risk associated with the disease.

In another aspect, there is disclosed a method of prognosing or classifying a patient comprising: determining mRNA abundance using a sample of a breast cancer tumour of the patient for the group of genes comprising: GSK3B, AKT1S1, RHEB, TSC1, TSC2, RPS6KB1, RPTOR, MTOR, RICTOR, ERBB2, MKI67, ESR1 and PGR, each of said genes associated with at least one node of the PIK3 cell signalling pathway; constructing an expression profile from the mRNA abundance; comparing said expression profile to a plurality of reference expression profiles and comparing clinical indicators of the patient to a plurality of reference clinical indicators, wherein the clinical indicators comprise N-stage and tumour size, and wherein each of the plurality of reference expression profiles and each of the reference clinical indicators are associated with a predetermined residual risk of breast cancer; and selecting the reference expression profile most similar to the expression profile and the reference clinical indicators most similar to the patient clinical indicators, to obtain a residual risk associated with breast cancer.

In yet another aspect, there is disclosed a computer-implemented method of prognosing or classifying a patient using a biomarker comprising a plurality of subnetwork modules, said method comprising: storing, in electronic memory, a model for predicting patient outcomes for patients having a disease, the model trained with a plurality of reference dysregulation scores and a plurality of reference clinical indicators; receiving, at at least one processor, data reflecting an activity of a plurality of genes in a test sample of the patient, said plurality of genes associated with the plurality of subnetwork modules; constructing, at the at least one processor, an expression profile using the data reflecting the activity of the plurality of genes; determining, at the at least one processor, dysregulation of each of the plurality of subnetwork modules by calculating a score proportional to a degree of dysregulation in each of the plurality of subnetwork modules from said expression profile; prognosing or classifying, at the at least one processor, the patient by: inputting each dysregulation score into the model; and inputting a clinical indicator of the patient into the model to obtain a risk associated with the disease.

In one aspect, there is disclosed a computer-implemented method of prognosing or classifying a patient, the method comprising: receiving, at at least one processor, data reflecting mRNA abundance determined using a sample of a breast cancer tumour of the patient for the group of genes comprising: GSK3B, AKT1S1, RHEB, TSC1, TSC2, RPS6KB1, RPTOR, MTOR, RICTOR, ERBB2, MKI67, ESR1 and PGR, each of said genes associated with at least one node of the PIK3 cell signalling pathway; constructing, at the at least one processor, an expression profile from the data reflecting mRNA abundance; comparing, at the at least one processor, said expression profile to a plurality of reference expression profiles and comparing clinical indicators of the patient to a plurality of reference clinical indicators, wherein the clinical indicators comprise N-stage and tumour size, and wherein each of the plurality of reference expression profiles and each of the reference clinical indicators are associated with a predetermined residual risk of breast cancer; and selecting, at the at least one processor, the reference expression profile most similar to the expression profile and the reference clinical indicators most similar to the patient clinical indicators, to obtain a residual risk associated with breast cancer.

In one aspect, there is disclosed a device for prognosing or classifying a patient using a biomarker comprising a plurality of subnetwork modules, the device comprising: at least one processor; and electronic memory in communication with the at least one processor, the electronic memory storing: a model for predicting patient outcomes for patients having a disease, the model trained with a plurality of reference dysregulation scores and a plurality of reference clinical indicators; and processor-executable code that, when executed at the at least one processor, causes the at least one processor to: receive data reflecting an activity of a plurality of genes in a test sample of the patient, said plurality of genes associated with the plurality of subnetwork modules; construct an expression profile using the data reflecting the activity of the plurality of genes; determine dysregulation of each of the plurality of subnetwork modules by calculating a score proportional to a degree of dysregulation in each of the plurality of subnetwork modules from said expression profile; prognose or classify the patient by: inputting each dysregulation score into the model; and inputting a clinical indicator of the patient into the model to obtain a risk associated with the disease.

In another aspect, there is disclosed a device for prognosing or classifying a patient, the device comprising: at least one processor; and electronic memory in communication with the at one processor, the electronic memory storing processor-executable code that, when executed at the at least one processor, causes the at least one processor to: receive data reflecting mRNA abundance determined using a sample of a breast cancer tumour of the patient for the group of genes comprising: GSK3B, AKT1S1, RHEB, TSC1, TSC2, RPS6KB1, RPTOR, MTOR, RICTOR, ERBB2, MKI67, ESR1 and PGR, each of said genes associated with at least one node of the PIK3 cell signalling pathway; construct an expression profile from the data reflecting mRNA abundance; compare said expression profile to a plurality of reference expression profiles and comparing clinical indicators of the patient to a plurality of reference clinical indicators, wherein the clinical indicators comprise N-stage and tumour size, and wherein each of the plurality of reference expression profiles and each of the reference clinical indicators are associated with a predetermined residual risk of breast cancer; and select the reference expression profile most similar to the expression profile and the reference clinical indicators most similar to the patient clinical indicators, to obtain a residual risk associated with breast cancer.

In another aspect, there is disclosed a method of treating a patient, comprising: determining the disease relapse risk of the patient according to the methods disclosed herein; and selecting a treatment based on the disease relapse risk, and preferably treating the patient according to the treatment.

In yet another aspect, there is disclosed a computer-implemented method of constructing a biomarker for a biological state of a given type, the method comprising: maintaining an electronic datastore storing: a plurality of subnetwork records, each comprising data reflecting one of a plurality of subnetwork modules of biological pathways; and a plurality of patient records, each comprising data reflecting molecular aberration measured for one of a plurality of patients of the biological state, and data reflecting a patient state for that patient; processing, at at least one processor, the subnetwork records and the patient records to assign, to each of the plurality of subnetwork modules, a score proportional to a degree of dysregulation in that subnetwork module; ranking, at the at least one processor, the plurality of subnetwork modules according to score assigned to each of the plurality of subnetwork modules; and upon said ranking, selecting, at the at least one processor, the biomarker as comprising a subset of the plurality of subnetwork modules.

In one aspect, there is disclosed a computer-implemented method of identifying a dysregulated subnetwork module of a biological pathway causing a biological state of a given type, the method comprising: maintaining an electronic datastore storing: a plurality of subnetwork records, each comprising data reflecting one of a plurality of subnetwork modules of biological pathways; and a plurality of patient records, each comprising data reflecting molecular aberration measured for one of a plurality of patients of the biological state, and data reflecting a patient state for that patient; processing, at at least one processor, the subnetwork records and the patient records to assign, to each of the plurality of subnetwork modules, a score proportional to a degree of dysregulation in that subnetwork module; identifying, at the at least one processor, from the scores, the dysregulated subnetwork module from amongst the plurality of subnetwork modules.

In yet another aspect, there is disclosed a device for constructing a biomarker for a biological state of a given type, the device comprising: at least one processor; and electronic memory in communication with the at least one processor, the electronic memory storing: a plurality of subnetwork records, each comprising data reflecting one of a plurality of subnetwork modules of biological pathways; a plurality of patient records, each comprising data reflecting molecular aberration measured for one of a plurality of patients of the biological state, and data reflecting a patient state for that patient; and processor-executable code that, when executed at the at least one processor, causes the at least one processor to: process the subnetwork records and the patient records to assign, to each of the plurality of subnetwork modules, a score proportional to a degree of dysregulation in that subnetwork module; rank the plurality of subnetwork modules according to score assigned to each of the plurality of subnetwork modules; and upon said ranking, select the biomarker as comprising a subset of the plurality of subnetwork modules.

In one aspect, there is disclosed a device for identifying a dysregulated subnetwork module of a biological pathway causing a biological state of a given type, the device comprising: at least one processor; and electronic memory in communication with the at least one processor, the electronic memory storing a plurality of subnetwork records, each comprising data reflecting one of a plurality of subnetwork modules of biological pathways; a plurality of patient records, each comprising data reflecting molecular aberration measured for one of a plurality of patients of the biological state, and data reflecting a patient state for that patient; and processor-executable code that, when executed at the at least one processor, causes the at least one processor to: process the subnetwork records and the patient records to assign, to each of the plurality of subnetwork modules, a score proportional to a degree of dysregulation in that subnetwork module; identify from the scores, the dysregulated subnetwork module from amongst the plurality of subnetwork modules.

In another aspect, there is disclosed a system comprising: a first device for prognosing or classifying a patient using a biomarker comprising a plurality of subnetwork modules; a second device for constructing a biomarker for a biological state of a given type, the device comprising; and wherein the biomarker of the first device is a biomarker constructed by the second device.

### BRIEF DESCRIPTION OF THE DRAWINGS

In the drawings, embodiments are illustrated by way of example. It is to be expressly understood that the description and drawings are only for the purpose of illustration and as an aid to understanding, and are not intended as a definition of the limits of the invention.

Embodiments will now be described, by way of example only, with reference to the attached figures, wherein:

FIG. 1 is a network diagram showing a biomarker construction/pathway identification device and a patient prognosis/classification device, interconnected by a computer network, exemplary of an embodiment;

FIG. 2 is a high-level schematic diagram of the hardware components of the biomarker construction/pathway identification device of FIG. 1;

FIG. 3 is a high-level schematic diagram of the software components of the biomarker construction/pathway identification device of FIG. 1, including a biomarker construction/pathway identification application, exemplary of an embodiment;

FIG. 4 is a high-level block diagram of the components of the biomarker construction/pathway identification application of FIG. 3;

FIG. 5 is a high-level schematic diagram of the hardware components of the patient prognosis/classification device of FIG. 1;

FIG. 6 is a high-level schematic diagram of the software components of the patient prognosis/classification of FIG. 1, including a patient prognosis/classification application, exemplary of an embodiment;

FIG. 7 is a high-level block diagram of the components of the patient prognosis/classification application of FIG. 6;

FIG. 8 shows heatmaps providing an overview of cohort and datasets of the PIK3 signalling pathway. Heatmaps show mRNA abundance for each gene in each module of the PI3K pathway as z-scores. Columns are patients, ordered by DRFS event status (top bar) with black representing an event and white representing no event. Univariate survival modelling in the training cohort for genes and clinical variables (HER2, age, grade, nodal status and pathological tumor size) is presented as forest plots (right; square represents hazard ratios; ends of the lines represent 95% confidence intervals). Mutational profiles of AKT1, PIK3CA and RAS (HRAS, KRAS, NRAS) were categorized into non-synonymous mutant and wild-type groups;

FIG. 9 provides prognostic and risk outcomes associated with IHC4-derived prognostic models. (A) Risk prediction by the IHC4 protein model in the validation cohort. Quartiles were defined in the training cohort and applied to the validation cohort. Quartiles Q2-Q4 were compared against Q1, with adjustment for age, Nodal status, tumor size and grade using Cox proportional hazards modelling and the log-rank test. (B) Comparison between predicted risk-scores of IHC4-mRNA and IHC4-protein models using Spearman's rank correlation, rho (p). Histograms show the distribution of risk scores derived using RNA (top) and protein (right) data respectively. (C) Validation of mRNA abundance-based multivariate prognostic model trained on ESR1, PGR, ERBB2 and MKI67 with statistical analysis as in (A);

FIG. 10 provides module dysregulation profiles associated with the PIK3 signalling pathway. (A) Correlation (Spearman's p) between per-patient MDSs in the training cohort. (B) Patient MDS stratified by AKT1 and PIK3CA mutation status. The boxplots show the distribution of MDS in wild-type AKT1 and PIK3CA (white boxes), and with either AKT1 mutation or PIK3CA mutations (black boxes). Statistical significance was estimated using a one-way ANOVA with correction for multiple comparisons using the Benjamini & Hochberg method. (C) A schematic view of the PI3K signalling pathway illustrating the key relationships between modules assessed in the current study. Modules 1-7 are highlighted with key signalling inter-relationships between genes illustrated;

FIG. 11 provides prognostic outcomes associated with the Modules-derived prognostic model of the present disclosure. (A) Independent validation of prognostic model trained on MDS and clinical covariates (N and tumor size). Risk score estimates were grouped into quartiles derived from the TEAM training cohort; each group was compared against Q1. Hazard ratios were estimated using Cox proportional hazards model and significance estimated using the log-rank test. (B) Independent validation of prognostic model in (A) stratified by PIK3CA mutations. Patients were classified into low- and high-risk groups, and these were then divided by PIK3CA mutant (+) and wild-type (−) mutation status. (C) Distribution of patient risk scores in the TEAM Validation cohort (top panel). Bottom panel shows the predicted 5-year recurrence probabilities (solid line) and 95% Cl (dashed lines) as a function of patient risk score. Vertical dashed black line indicates training set median risk score. (D) Comparison of MDS model, IHC4-mRNA and IHC4-protein models using area under the receiver operating characteristic (AUC) curve as performance indicator;

FIG. 12 shows power calculation methods in the TEAM cohort. Power calculation for hazard ratios (HR) ranging from 1 to 3 for complete TEAM cohort as well as Training and Validation cohorts separately. Dashed line (power=0.8) represents a threshold of minimum 80% power for each of the three cohort groups;

FIG. 13 is a schematic view of the PI3K signaling pathway illustrating some of the key relationships between modules assessed in the current disclosure;

FIG. 14 depicts preprocessing results associated with the TEAM cohort. (A) Density plots show the distribution of Spearman's rank correlation coefficients estimated for the RNA profiles grouped into pooled and clinical samples. The intra-pooled correlations (yellow distribution) indicate almost perfect correlation, reflecting minimal sample processing artefacts. (B) Heatmap shows ranking of preprocessing methods based on their ability to maximise molecular differences between HER2+ and HER2-profiles, while minimizing batch effects. For 252 combinations of preprocessing methods, two rankings were established as per above criteria, and subsequently aggregated using the rank product. The heatmap is sorted on the aggregate rank with the most effective preprocessing parameters at the top;

FIG. 15 shows mRNA abundance profiles of the TEAM cohort using heatmaps showing the normalized and scaled mRNA abundance profiles of the TEAM cohort, Training and Validation combined. Both patients (rows) and genes (columns) were clustered using 1-Pearson's correlation as the distance measure followed by Ward hierarchical clustering. Row covariates represent the HER2 status determined through IHC (green=positive, white=negative, gray=NA);

FIG. 16 provides data relating to IHC4-derived prognostic models. (A) Validation of IHC415 protein model using ER, PgR, HER2 (+/−) and Ki67 markers in TEAM Training cohort. IHC4 risk scores were classified into quartiles. Groups Q2-Q4 were compared against Q1, followed by adjustment for age, Nodal status, tumour size and grade. Hazard ratios were estimated using Cox proportional hazards modelling with significance evaluated using the log-rank test. (B) Comparison between predicted risk-scores of IHC4-mRNA and IHC4-protein models. Correlation rho (p) represents Spearman's rank correlation coefficient. Histograms show the distribution of risk scores derived using RNA (top) and protein (right) data respectively. (C) Prognostic assessment of mRNA abundance-based multivariate prognostic model trained on ESR1, PGR, ERBB2 and MKI67;

FIG. 17 demonstrates IHC4-RNA predicted risk scores. (A) Distribution of patient risk scores in the TEAM Training cohort (top panel). Bottom panel shows the predicted 5-year recurrence probabilities (solid lines) and 95% Cl (dashed lines) as a function of patient risk score. (B) Same as A except the risk scores shown are from the TEAM Validation cohort;

FIG. 18 provides data relating to Module dysregulation profiles. (A) Correlation (Spearman's Rho) between per-patient module dysregulation scores (MDS) in the TEAM Validation cohort. (B) Patient MDS stratified by AKT1 and PIK3CA mutation status. The boxplots show the distribution of MDS in wild-type AKT1 and PIK3CA (white boxes), and with either AKT1 mutation or PIK3CA mutations (black boxes). Statistical significance was estimated using a one-way ANOVA. P values were corrected for multiple comparisons using Benjamini & Hochberg method;

FIG. 19 is a representation of the outcomes associated with the Modules-derived prognostic model associated with the PIK3 signalling pathway. (A) Prognostic model trained on MDS and clinical covariates (N-stage and tumour size). Risk score estimates were grouped into quartiles; each group was compared against Q1. Hazard ratios were estimated using Cox proportional hazards model and significance estimated using the log-rank test. (B) Prognostic assessment of model in (A) stratified by PIK3CA mutations. Patients were classified into low- and high-risk groups, and each was further divided by PIK3CA mutant (+) and wild-type (−) status. (C, D) Prognostic assessment of model in (A) by median-dichotomizing predicted risk scores into low- and high-risk groups. (E) Distribution of patient risk scores in the TEAM Training cohort (top panel). Bottom panel shows the predicted 5-year recurrence probabilities (solid lines) and 95% Cl (dashed lines) as a function of patient risk score. Modules-derived prognostic model predicts higher likelihood of recurrence for patients with higher risk score. Vertical dashed black line indicates training set median risk score. (F, G) Same as E, however, with predicted 10-year recurrence probabilities. (H) Performance comparison of MDS model versus IHC4-RNA and IHC4-protein models using area under the receiver operating characteristic (ROC) curve (AUC) as performance indicator. AUC of MDS model significantly exceeded both IHC4-RNA and IHC4-protein models;

FIG. 20 is a schematic overview of SIMMS. Subnetwork modules are extracted from NCI-Nature/Biocarta/Reactome curated pathways by isolating protein-protein interaction networks within a pathway. Molecular profiles are systemised and split into independent training and validation sets. Each extracted subnetwork is scored (module-dysregulation score) using 3 different models and ranked. High-ranking subnetworks are used to compute a patient-wise risk-score. Most optimal combination of predictive subnetworks is selected using Backward elimination and Forward selection algorithms, resulting in a multivariate subnetwork-based classifier. The classifier is then tested on the validation sets independently as well as on combined validation set;

FIG. 21 depicts heatmaps which reveal co-regulated pathways. (A) Highly prognostic subnetwork markers in breast cancer. Kaplan-Meier analysis of risk groups determined by univariate analysis of per-patient MDS in the validation cohort. (B,C) Heatmap of correlation and cluster analysis of patient's MDS across top n_{Breast}=50, n_{NSCLC}=25 subnetwork markers. Red bars across the axes indicate highly correlated clusters of subnetwork modules;

FIG. 22 is a representation of the degree of overlap between cancer biomarkers. (A) Overlap of candidate subnetwork markers across breast, colon, NSCLC (non-small cell lung cancer) and ovarian cancers. (B) Univariate prognostic evaluation of overlapping modules within the validation cohorts of the respective cancer type. (C) Cross cancer correlation plot (Spearman) of subnetwork modules' performance of all sampled biomarkers (Methods). Correlation was estimated on the Cox proportional hazards model's coefficient (β) in absolute scale. (D) Performance of breast, colon, NSCLC and ovarian cancer candidate biomarkers represented as a function of size. These randomization results depict a range of prognostic performance between 75th and 95th percentiles at each marker size and were used as a guide to estimate the most optimal top n number of subnetwork modules required to establish a classifier for a given tumour type.

FIG. 23 shows mRNA-based biomarkers for multiple tumour types (A-D) Kaplan-Meier survival plots using Model N over the entire validation cohort with subnetwork module selection conducted using forward selection algorithm. Using AIC metric iteratively, the stepwise model selection resulted in 17/50, 8/75, 6/25 and 14/50 subnetwork modules for breast, colon, NSCLC and ovarian cancers respectively (Tables 18-21).

FIG. 24 is a clinical analysis of breast cancer biomarkers. (A) Heatmap of correlation and cluster analysis of patients' MDS profiles of top nBreast=50 subnetwork modules in the Metabric validation cohort. The covariates demonstrate PAM50-based molecular subtypes along with SIMMS predicted risk group. (B) Forest plot showing HR and 95% Cl (multivariate Cox proportional hazards model) of the analyses of Metabric dataset. Datasets originating from Illumina (ILMN) and Affymetrix (AFFY) were used for cross platform training and validation purposes. Due to limited availability of clinical annotations, only the Illumina dataset (Metabric) was used for subtype-specific models. For these, the Metabric-published training and validation cohorts were maintained, except for Her2-positive and Normal-like breast cancer subtypes where the Metabric training and validation cohorts were reversed due to relatively small number of patients in the training set. Numbers in parenthesis indicate the size of the validation cohort. Asterisks represent statistical significance of differential outcome between the predicted low- and high-risk groups (* p<0.05, ** p<0.01, *** p<0.001);

FIG. 25 shows multimodal prognostic biomarkers for breast and ovarian cancer. (A, B, C) Kaplan-Meier survival analysis of SIMMS predictions on the Metabric validation cohort. Using Metabric training cohort, three models were trained on CNA and mRNA profiles. As indicated in (C), CNA and mRNA profiles taken together better predicted patient prognosis compared to either of these modeled alone. (D) Permutation analysis of TOGA ovarian cancer dataset. The bar plot shows the mean of absolute hazard ratios (HR) in log₂-scale estimated over 1,000 iterations. For each permutation of training and validation datasets, 7 different classifiers were established using CNA, mRNA and DNA methylation profiles. Asterisks represent statistical significance of difference in the HRs between the models (*** p<0.001 for all comparisons indicated; Welch's unpaired t-test);

FIG. 26 are a set of graphs which show (a,b) the distribution of nodes and edges across all subnetwork modules extracted from NCI-Nature curated pathways;

FIG. 27 depicts the results of (a,b,c) a univariate Cox model that was fit to each gene in each study in the breast cancer cohort. Genes were ranked according to their p value (Wald-test), and a cumulative rank for all the genes was estimated using the rank product for each gene. The top ranked 100 (a), 500 (b) and 1,000 (c) genes were used to identify the study in which each gene was farthest away from the cumulative rank. The frequency of a study being farthest was recorded for each of the top ranked 100, 500 and 1,000 genes. Li and Loi datasets seem to be notable outliers. As the threshold is relaxed, Sabatier dataset also begins to show deviation compared to other datasets; (d) The heatmap shows a summary of barplots (a-c) of the top ranked (rank product) 100 to 2000 genes with the percentage measure as the frequency of each dataset being the farthest from the rank product of top n genes. The covariates represent different array platforms. These are: HG-U95AV2=purple, HTHG-U133A=green, HG-U133A=red, HG-U133-PLUS2=yellow; (e) 4-way Venn diagram representing overlap of genes across the four Affymetrix array platforms used in the 14 breast cancer datasets included in this study. Note that the Bild dataset (array platform: HG-U95AV2) has the least number of genes (8,260) with 8,052 genes that exist across all array platforms. The analysis in a-d was done on this common gene set only; (f,g,h) The gene ranks were transformed into percentile ranks within all studies. The rank product based top 100 (f), 500 (g), and 1,000 (h) genes shown in terms of their percentile rank within each study. Li, Loi and Chin datasets seem to cluster together and have lower percentile ranks compared to other datasets. However, Sabatier shows percentile ranks similar to other datasets thereby removing doubts of being an outlier; (i) Summary heatmap of percentile ranks across all studies, ordered by groups of genes common across studies, thereby maintaining coherent comparison of ranks; (j) Heatmap of Spearman correlation between patients' mRNA abundance profiles. Loi dataset quite clearly shows weak correlation with the other datasets, again reflecting unusual behaviour compared to other datasets; (k,l) Box-whisker plots of intra-(k) and inter-study (l) correlation between patients' mRNA abundance profiles. The results show distinctively strong correlation within Loi dataset (k) and weak correlation between Loi and other datasets (l); (m) Histogram of Spearman correlation of patients' mRNA abundance profiles. From left to right, the first peak represents correlation between Loi and other datasets. The second peak represents correlation between Bild and other datasets, while the third peak constitutes the correlation between the remaining datasets. The survival data of highly correlated profiles (zoomed in panel, 0.98≦ρ≦1.00) was further inspected, resulting in 22 patients that were found in both Sotiriou and Symmans (JBI) datasets having identical survival data. These were removed from Symmans (JBI) dataset for further analysis;

FIG. 28 shows the distribution of low- and high-scoring nodes (N_{LS}, N_{HS}) and edges (E_{LS}, E_{HS}) in top n (n_{Breast}=50, n_{Colon}=75, n_{NSCLC}=25 and n_{Ovarian}=50) subnetworks using MDS of Model N. The significance of difference between each set of nodes (N_{LS} & N_{HS}) and edges (E_{LS} & E_{HS}) was computed using bootstrapping with 100,000 iterations (P<10⁻³ for all eight pairs);

FIG. 29 shows the hazard ratios of gene signatures as a function of signature size across breast cancer, colon cancer, ovarian cancer and NSCLC. Jackknifing was performed over the subnetwork marker space for various tumour types. Ten million unique markers (200,000 for each marker size n=5, 10, 15, . . . , 250) were randomly sampled using all 500 subnetworks. The prognostic performance of each candidate biomarker was measured by taking the absolute value of the log₂-transformed hazard ratio estimated with a multivariate Cox proportional hazards model using each of the three module scoring methods implemented by SIMMS (Model N, Model E and Model N+E). Each panel shows the range of hazard ratios between the 75th and 95th percentiles at each marker size for the four tumour types, along with the hazard ratios of the subnetwork markers chosen by the SIMMS feature selection algorithms (backward elimination and forward selection);

FIG. 30 depicts the null distribution of SIMMS's Model N for selected signature sizes of (a) n=25, (b) n=50 and (c) n=75. Ten million random permutations of subnetworks were generated (n₂₅=4 million, n₅₀=4 million and n₇₅=2 million). Prognostic classifiers of breast, colon, NSCLC and ovarian were created for each permutation. The prognostic performance of these classifiers was measured by taking the absolute value of the log₂-transformed hazard ratio estimated using a multivariate Cox proportional hazards model (forward selection);

FIG. 31 shows (a) Box-Whisker plots of p-values (Wald test) for each of the three models. Pair-wise comparison for significance of difference was done using Wilcoxon rank-sum test. (b) Box-Whisker plots of bootstrap analysis (n=10,000) for each of the three subnetwork models (N, E, and N+E) followed by training prognostic models using forward selection algorithm (Methods). The results compared here are the estimated hazard ratios between the SIMMS's predicted risk groups in the independent validation cohort;

FIG. 32 depicts volcano plots of hazard ratios (with 95% Cl) for each of the top n subnetwork modules following Cox proportional hazards model fitted to dichotomous risk scores across the entire validation cohort. The asymmetric nature of the volcano plots is a property of modelling MDS as a magnitude of gene's predictive estimate (HR).

FIG. 33 is a Venn diagram showing overlapping genes between subnetwork modules derived from the pathways of Aurora A signaling (module 1), Aurora B signaling (module 1) and PLK1 signaling events (module 1). The single gene common across all three pathways was AURKA. The module number corresponds to the subnetwork number of a given pathway

FIG. 34 is a heatmap of correlation and cluster analysis of patients' MDS across top ranked 75 subnetwork markers of colon cancer (validation datasets only). Red bars across the axes indicate highly correlated clusters of subnetwork modules;

FIG. 35 is a heatmap of correlation and cluster analysis of patients' MDS across top ranked 50 subnetwork markers of ovarian cancer (validation datasets only). Red bars across the axes indicate highly correlated clusters of subnetwork modules;

FIG. 36 shows the performance of each of Models N, E and N+E using backward elimination and forward selection. Patients were dichotomized into naïve low- and high-risk groups by using 8, 6, 3 and 3 years survival status as cut-off for breast, colon, NSCLC and ovarian cancers respectively. The naïve grouping was compared to SIMMS's predicted risk groups to compute confusion table and percentage prediction accuracy. Both feature selection approaches suggest similar accuracy implying SIMMS's insensitivity towards these two feature selection algorithms;

FIG. 37 shows Kaplan-Meier survival plots using SIMMS's Model N on 6 breast cancer validation sets (Table 10) individually (10-year survival truncation) with subnetwork module selection conducted using forward selection (top two rows) and backward elimination (bottom two rows) algorithm. Both feature selection algorithms were initialized with the top ranked 50 subnetwork markers. The results of the two feature selection approaches were found fairly consistent;

FIG. 38 shows Kaplan-Meier survival plots using SIMMS's Model N on 2 colon cancer validation sets (Table 11) individually (6-year survival truncation) with subnetwork module selection conducted using forward selection (top row) and backward elimination (bottom row) algorithm. Both feature selection algorithms were initialized with the top ranked 75 subnetwork markers;

FIG. 39 shows Kaplan-Meier survival plots using SIMMS's Model N on 6 NSCLC cancer validation sets (Table 12) individually (5-year survival truncation) with subnetwork module selection conducted using forward selection (top two rows) and backward elimination (bottom two rows). Both feature selection algorithms were initialized with the top ranked 25 subnetwork markers;

FIG. 40 shows Kaplan-Meier survival plots using SIMMS's Model N on 3 ovarian cancer validation sets (Table 13) individually (5-year survival truncation) with subnetwork module selection conducted using forward selection (top row) and backward elimination (bottom row). Both feature selection algorithms were initialized with the top ranked 50 subnetwork markers;

FIG. 41 shows Kaplan-Meier survival plots using Model N over the entire validation cohort with subnetwork module selection conducted using backward elimination;

FIG. 42 shows Kaplan-Meier survival plots of SIMMS's Model N based predictions on the Metabric validation cohort. The classifiers were established using the Affymetrix based breast cancer training cohort (Table 10) as well as Illumina based breast cancer cohort (Metabric training set). Both classifiers were applied to predict risk group in the Metabric validation cohort, which were assessed for survival association using Kaplan-Meier survival analysis.

### DETAILED DESCRIPTION

As a consequence of the complexity of human disease, disease researchers face two pressing challenges. First, molecular markers are needed to personalize and optimize treatment decisions by predicting patient outcome (prognosis) and response to therapy. Second, the clinical heterogeneity in patient outcome needs to be molecularly rationalized to allow direct targeting of the mechanistic underpinnings of disease. For example, if a single pathway is being dysregulated in multiple ways, drugs targeting that pathway as a whole could be developed. Further, there is a need for improved ways to detect or predict various other aspects of patient state such as disease type, disease subtype, cancer type, cancer subtype, disease state, or the like.

Conventionally, most validated multigene tests for residual risk prediction in breast cancer were generated using genome-wide analysis of mRNA data and are strongly driven by proliferation [5]. They provide similar and modest clinical utility [6, 7], do not identify key pathways for targeted therapeutics and do not inform patients or clinicians on the optimal therapeutic approach. One alternative is to use key signaling pathways to improve the accuracy of multi-parameter tests for residual risk prediction and to stratify patients into trials of targeted molecular therapeutics. The PIK3CA signalling pathway represents a robust candidate for this approach as it is frequently dysregulated in multiple cancer types [8], including breast cancer [9-12]. Mutations in PIK3CA are present in almost 40% of luminal breast cancers [8, 9, 13, 14] and drugging of the PIK3CA/mTOR pathway is a promising approach for advanced breast cancer [15]. Nonetheless, to date mutational analysis of the PIK3CA pathway has not enabled molecular targeting of existing agents, nor have key mechanistic events been identified in primary patients to focus drug development on specific pathway components [16-19].

In an aspect, this disclosure provides novel molecular markers and methods of prognosing or classifying a patient using such molecular markers.

For example, targeted molecular profiling was performed of the PIK3CA pathway in a multinational phase III clinical trial. These data allowed for the development and validation of a novel residual risk signature that out-performs a clinically-validated test.

In other aspects, the residual risk signature and associated methods developed in respect of breast cancer may be modified to provide prognostic signatures for a multitude of diseases, including colon, ovarian and lung cancers, and other biological states.

In another aspect, this disclosure also provides methods of using the novel breast cancer signature to stratify patients for trials targeting PIK3CA signaling nodes. More generally, this disclosure provides methods of using the signatures detailed herein to stratify patients for particular trials/treatments that target particular pathways and/or particular nodes/edges of those pathways.

In a further aspect, a subnetwork-based approach is provided that can use arbitrary molecular data types to identify one or more dysregulated pathways and to create functional biomarkers for a variety of biological states (e.g., phenotypes, diseases of a given type, cancers of a given type, etc.).

In a yet further aspect, a subnetwork-based approach is used to identify one or more dysregulated pathways in order to stratify patients for trials/treatments that target those pathways or particular nodes/edges of those pathways.

In this disclosure, the terms “pathways” and “biological pathways” are used broadly to refer to cellular signaling pathways, extra-cellular signaling pathways, or other biological functional units such as protein complexes. “Pathways” or “biological pathways” may also refer to interaction amongst or between intra-cellular and/or extra-cellular molecules.

While there are several well-studied complex diseases, including Alzheimer's, schizophrenia and diabetes, examples are provided herein for cancer, as it is among the most heterogeneous complex disease [63, 64]. Patients with the same cancer type have highly variable outcome [65], response to therapy [66] and mutational profiles [67, 68]. Studies across multiple cancer types provide strong evidence that cancer mutations are often exclusive: exactly one gene in a pathway is dysregulated, leading to a common phenotype [69]. We validate the ability of our approach, called SIMMS, by using it to create prognostic models in cohorts of 4,096 breast, 517 colon, 749 lung and 1,303 ovarian cancer patients profiled with a diverse range of molecular assays.

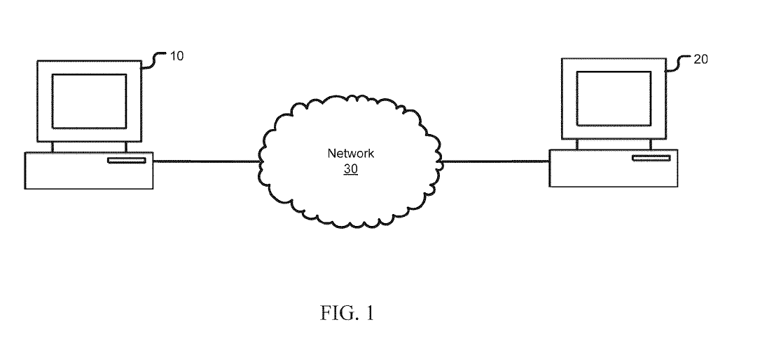

FIG. 1 depicts a system including a biomarker construction/pathway identification device 10 and a patient prognosis/classification device 20, exemplary of an embodiment. As will be detailed herein, biomarker/pathway identification device 10 is configured to construct biomarkers for given biological states. Biomarker construction/pathway identification device 10 may also be configured to identify a dysregulated cell signaling pathway resulting in given biological states. As will also be detailed herein, patient prognosis/classification device 20 is configured to perform prognosis and/or classification of patients using a biomarker (e.g., a disease).

As depicted, device 10 and device 20 may be interconnected by a network 30. When so interconnected, these devices may operate in concert to construct a biomarker for a given biological state, and then use that biomarker to perform prognosis and/or classifications of patients. In particular, biomarkers constructed by device 10 may be transferred to device 20, and used at device 20 to perform prognosis/classification in manners detailed herein. Of course, biomarkers constructed by device 10 may also be transferred to device 20 in other ways, e.g., by way of suitable computer storage/transport media (e.g., disks).

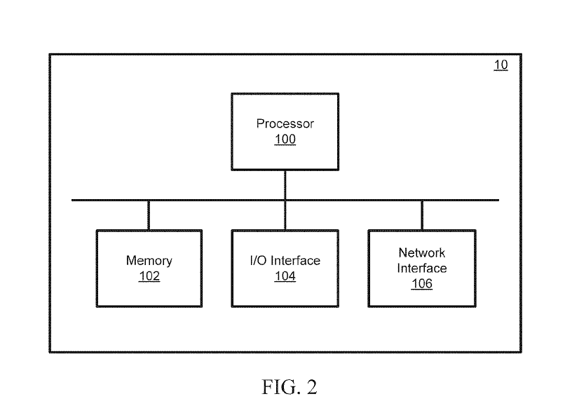

FIG. 2 depicts the hardware components of biomarker construction/pathway identification device 10, in accordance with an example embodiment. As depicted, device 10 includes at least one processor 100, memory 102, at least one I/O interface 104, and at least one network interface 106.

Processor 100 may be any type of processor, such as, for example, any type of general-purpose microprocessor or microcontroller (e.g., an Intel™ x86, PowerPC™, ARM™ processor, or the like), a digital signal processing (DSP) processor, an integrated circuit, a field programmable gate array (FPGA), or any combination thereof.

Memory 102 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), or the like. Portions of memory 102 may be organized using a conventional filesystem, controlled and administered by an operating system governing overall operation of device 10.

I/O interfaces 104 enable device 10 to interconnect with input and output devices. For example, I/O interfaces 104 may enable device 10 to interconnect with other input/output devices such as a keyboard, mouse, display, storage device, or the like.

Network interfaces 106 enable device 10 to communicate with other devices by connecting to one or more networks such as network 30 (FIG. 1).

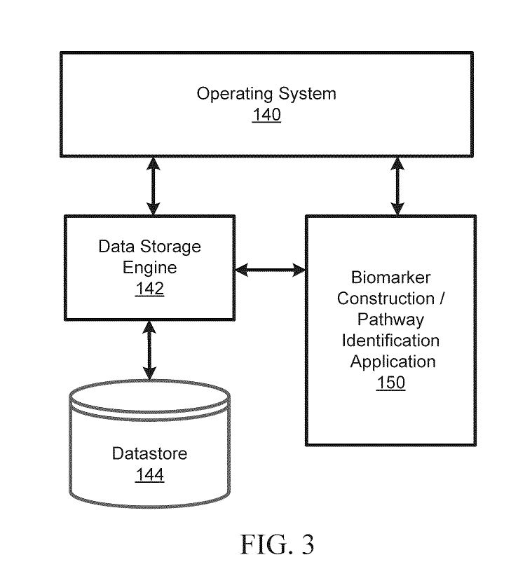

FIG. 3 depicts the software components of biomarker construction/pathway identification device 10, in accordance with an example embodiment. As depicted, device 10 includes an operating system 140, a data storage engine 142, a datastore 144, and a biomarker construction/pathway identification application 150. These software components may be stored in memory 102, and executed at processor(s) 100.

Operating system 140 may be a conventional operating system. For example, operating system 140 may be a Microsoft Windows™, Unix™, Linux™, OSX™ operating system or the like. Operating system 140 allows patient prognosis/classification application 150 and other applications at device 10 to access the hardware components of device 10 (e.g., processors 100, memory 102, I/O interfaces 104, network interfaces 106).

Data storage engine 142 allows operating system 140 and applications at device 10 to read from and write to datastore 144. Datastore 144 may be a conventional relational database such as a MySQL™, Microsoft™ SQL, Oracle™ database, or the like. So, data storage engine 142 may be a conventional relational database engine. Datastore 144 may also be another type of database such as, for example, an objected-oriented database or a NoSQL database, and data storage engine 142 may be a database engine adapted to read from and write to such other types of databases. Datastore 144 may reside in memory 102.

In some embodiments, datastore 144 may also simply be a collection of files stored and organized in memory 102. In such embodiments, data storage engine 142 may be omitted.

Datastore 144 may store a plurality of subnetwork records, each including data reflecting one of a plurality of subnetwork modules of one or more biological pathways.

Datastore 144 may also store a plurality of patient records, each including data reflecting molecular aberration measured for one of a plurality of patients of a biological state of a given type. The molecular aberration may include at least one of genomic aberration, epigenomic aberration, transcriptomic aberration, proteomic aberration, and metabolic aberration. More specifically, the molecular aberration may include at least one of somatic point mutation, small indel, mRNA abundance, somatic or germline copy-number status, somatic or germline genomic rearrangements, metabolite abundance, protein abundance, and DNA methylation.

Datastore 144 may also store a plurality of pathway records, each identifying a biological pathway associated with one of the plurality of subnetwork modules.

The records of datastore 144 may be populated by data retrieved from data repositories interconnected to device 10 by way of network interface 106, or by data inputted at device 10 through one of I/O interfaces 104.

As detailed herein, biomarker/pathway identification application 150 may be configured to implement the SIMMS approach detailed herein. As such, application 150 may also be referred to as “SIMMS” herein, or an application implementing “SIMMS”.

So, application 150 may be configured to implement methods of constructing a biomarker for a biological state of a given type, where the biomarker is selected as including a subset of a plurality of subnetwork modules. Application 150 may be also configured to implement methods of identifying a dysregulated subnetwork module of a biological pathway causing a biological state of a given type.

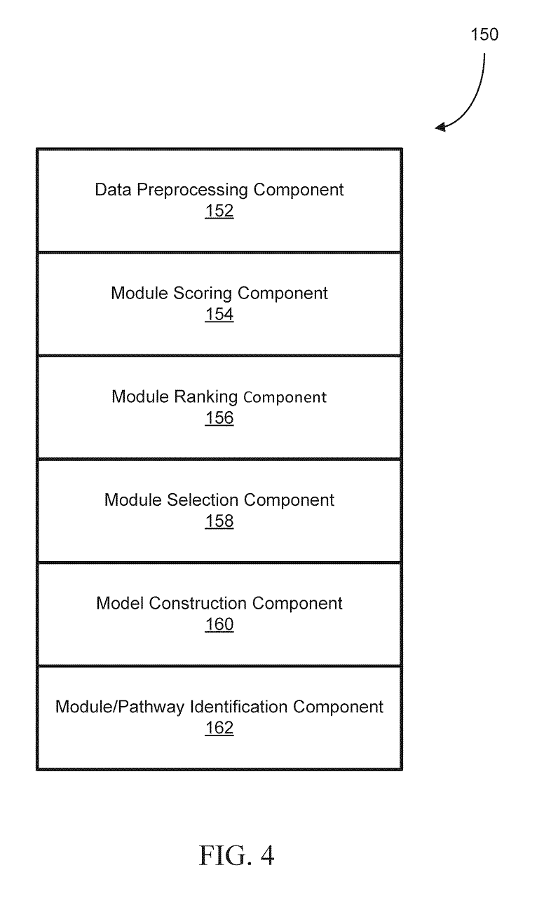

FIG. 4 depicts components of application 150, in accordance with an example embodiment. As depicted, application 150 includes a data preprocessing component 152, a module scoring component 154, a module ranking component 156, a module selection component 158, a model construction component 160, and a module/pathway identification component 162.

Each of these components may be implemented in a high-level programming language (e.g., a procedural language, an object-oriented language, a scripting language, or any combination thereof). For example, each of these components may be implemented using C, C++, C#, Perl, Java, or the like. Each of these components may also be implemented in assembly or machine language. Each of the components may be in the form of an executable program, a script, a statically linkable library, or a dynamically linkable library.

In a particular embodiment, one or more of the components of application 150 may be implemented in the R programming language.

Data preprocessing component 152 is configured to preprocess (e.g. normalize) data reflecting measurements of molecular aberrations. Data may be normalized by one or more of a plurality of methods, including using algorithmic controls or experimental controls. For example, with respect to experimental controls, data may be normalized with reference to corresponding data collected from a patient or a plurality of patients and stored in datastore 144. For example, mRNA abundance of a given set of genes of a patient may be normalized with reference to mRNA abundance of the same set of genes obtained from a sample of one or more different samples of the patient, or alternatively samples obtained from one or more different patients. mRNA abundance for a patient may also be normalized with reference to mRNA abundance of one or more specific control genes (i.e., reference genes) of the same patient, or one or more different patients (i.e., a reference patient), said control genes may be different to those being assessed for purposes of constructing a biomarker or prognosing/classifying a patient. Alternatively, the data may be normalized using an algorithmic control to mathematically manipulate data to remove noise, reduce variance and make data comparable across multiple experimental cohorts. Algorithmic controls may also enable normalization with reference to external data sets.

Module scoring component 154 is configured to process the subnetwork records and the patient records in datastore 144 to assign, to each of the subnetwork modules, a score proportional to a degree of dysregulation in that subnetwork module.

Module ranking component 156 is configured to rank the subnetwork modules according to their assigned scores.

Module selection component 158 is configured to select, as a biomarker, a subset of the subnetwork modules.

As detailed in the examples below, module selection component 158 may be configured to perform this selection by applying backward variable elimination. Module selection component 158 may also be configured to perform this selection by applying forward variable selection.

In some embodiments, module selection component 158 may be configured to select the biomarker such that the subnetwork modules in the subset of the plurality of subnetwork modules belong to one biological pathway.

Model construction component 160 is configured to a construct model for predicting patient states, where the model includes a selected subset of subnetwork modules.

In the examples detailed below, a Cox proportional hazards model is constructed by model construction component 160. However, model construction component 160 may also be configured to construct other types of models for predicting patient state, such as, a general linear model, a random forest model, a support vector machine model, a k-nearest neighbour model, a naïve Bayes model, or the like.

Module/pathway identification component 162 is configured to identify from the calculated scores a dysregulated subnetwork module.

These components of application 150 (or a subset thereof) may cooperate to implement methods detailed herein.

In particular, they may implement a method of constructing a biomarker for a biological state of a given type. The method including: maintaining an electronic datastore (e.g., datastore 144) storing: a plurality of subnetwork records, each comprising data reflecting one of a plurality of subnetwork modules of biological pathways; and a plurality of patient records, each comprising data reflecting molecular aberration measured for one of a plurality of patients of the biological state, and data reflecting a patient state for that patient. The method also includes processing (e.g., by module scoring component 154), at least one processor (e.g., processors 100), the subnetwork records and the patient records to assign, to each of the plurality of subnetwork modules, a score proportional to a degree of dysregulation in that subnetwork module. The method also includes ranking (e.g., by module ranking component 156), at the at least one processor, the plurality of subnetwork modules according to score assigned to each of the plurality of subnetwork modules; and upon said ranking, selecting (e.g., by module selection component 158), at the at least one processor, the biomarker as comprising a subset of the plurality of subnetwork modules.

The method may also include constructing (e.g., by model construction component 160), at the at least one processor, a model for predicting patient states for patients of the biological state, the model comprising the selected subset of the plurality of subnetwork modules.

The method may also include preprocessing (e.g., by data preprocessing component 152) the data reflecting molecular aberration, e.g., to normalize the data.

The components of application 150 (or a subset thereof) may also cooperate to implement a method of identifying a dysregulated subnetwork module of a biological pathway causing a biological state of a given type. The method including: maintaining an electronic datastore (e.g., datastore 144) storing: a plurality of subnetwork records, each comprising data reflecting one of a plurality of subnetwork modules of biological pathways; and a plurality of patient records, each comprising data reflecting molecular aberration measured for one of a plurality of patients of the biological state, and data reflecting a patient state for that patient. The method also includes processing (e.g., by module scoring component 154), at at least one processor, the subnetwork records and the patient records to assign, to each of the plurality of subnetwork modules, a score proportional to a degree of dysregulation in that subnetwork module. The method also includes identifying (e.g., by module/pathway identification component 162), at the at least one processor, from the scores, the dysregulated subnetwork module from amongst the plurality of subnetwork modules.

In some embodiments, said identifying comprises identifying a plurality of dysregulated subnetwork modules from amongst the plurality of subnetwork modules.

The method may also include maintaining in the electronic datastore a plurality of pathway records, each identifying a biological pathway associated with one of the plurality of subnetwork modules, and processing (e.g., by module/pathway identification component 162), at the at least one processor, the pathway records to identify a biological pathway associated with the dysregulated subnetwork module.

The method may also include preprocessing (e.g., by data preprocessing component 152) the data reflecting molecular aberration, e.g., to normalize the data.

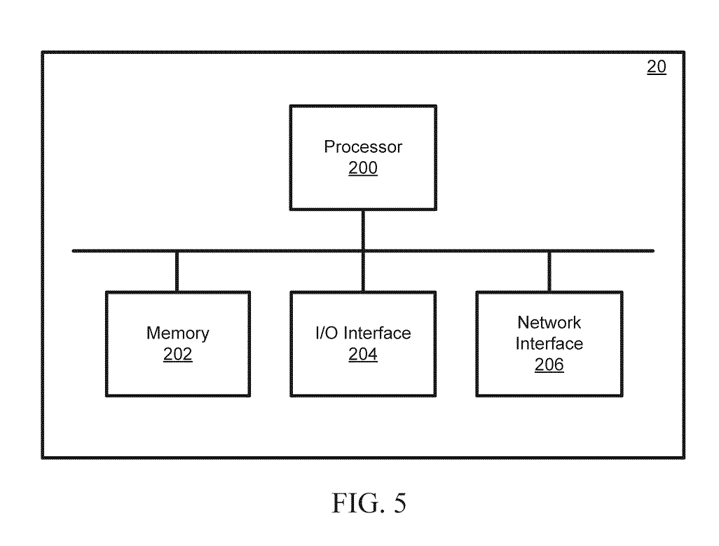

FIG. 5 depicts the hardware components of patient prognosis/classification device 20, in accordance with an example embodiment. As depicted, device 20 includes at least one processor 200, memory 202, at least one I/O interface 204, and at least one network interface 206. Processors 200 may be substantially similar to processors 100, memory 202 may be substantially similar to memory 102, I/O interfaces 204 may be substantially similar to I/O interfaces 104, and network interfaces 206 may be substantially similar to network interfaces 106.

I/O interfaces 204 enable device 20 to interconnect with input and output devices. For example, device 20 may be configured to receive patient data (e.g., mRNA abundance data) from an interconnected assay device, for example a gel electrophoresis device configured for northern blotting, a device configured for quantitative polymerase chain reaction (qPCR) or reverse transcriptase quantitative polymerase chain reaction (RT-qPCR), a hybridization microarray, a device configured for serial analysis of gene expression (SAGE), or a device configured for RNA Seq or Whole Transcriptome Shotgun Sequencing (WTSS), by way of I/O interface 204. I/O interfaces 204 also enable device 20 to interconnect with other input/output devices such as a keyboard, mouse, display, or the like.

Network interfaces 206 enable device 20 to communicate with other devices by connecting to one or more networks such as network 30 (FIG. 1).

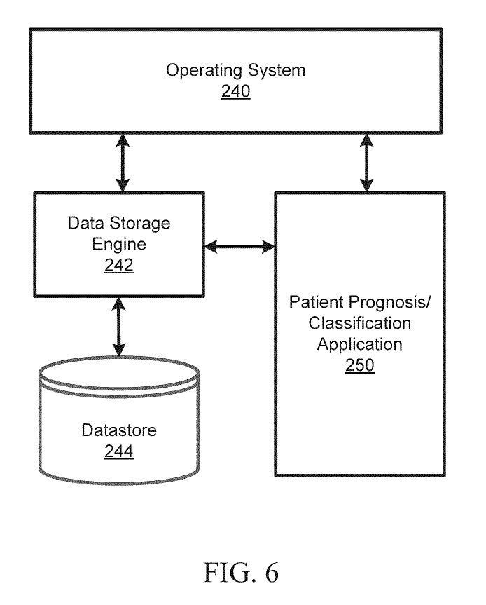

FIG. 6 depicts the software components of patient prognosis/classification 20, in accordance with an example embodiment. As depicted, device 20 includes an operating system 240, a data storage engine 242, a datastore 244, and a patient prognosis/classification application 250. These software components may be stored in memory 202, and executed at processor(s) 200.

Operating system 240 may be substantially similar to operating system 140. Operating system 240 allows biomarker/pathway identification application 250 and other applications at device 20 to access the hardware components of device 20 (e.g., processors 200, memory 202, I/O interfaces 204, network interfaces 206).

Data storage engine 242 may be substantially similar to data storage engine 142. Data storage engine 242 allows operating system 240 and applications at device 20 to read from and write to datastore 244.

Datastore 244 may store data reflective of measurements of molecular aberrations (e.g., mRNA abundance) obtained from a test sample, to be processed by application 150 in manners detailed below. Datastore 244 may also store one or more biomarkers to be used by application 250 in manners detailed below. Such biomarkers may be biomarkers constructed by biomarker construction/pathway identification device 10, and received therefrom.

The records of datastore 244 may be populated by data retrieved from data repositories interconnected to device 20 by way of network interface 206, or by data inputted at device 20 through one of I/O interfaces 204.

As detailed herein, patient prognosis/classification application 250 may be configured to perform prognosis and/or classification of patients using a biomarker for a given biological state, where the biomarker comprises a plurality of subnetwork modules.

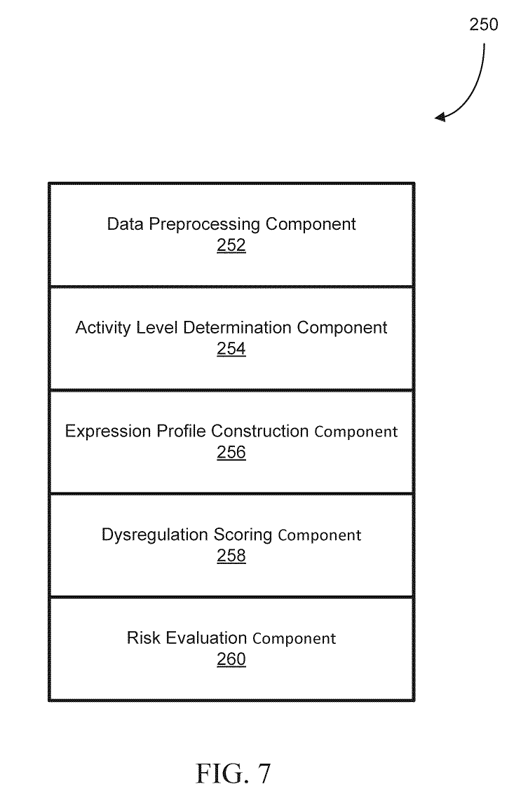

FIG. 7 depicts components of application 250, in accordance with an example embodiment. As depicted, application 250 includes a data preprocessing component 252, an activity level determination component 254, an expression profile construction component 256, a dysregulation scoring component 258, and a risk evaluation component 260.

Each of these components may be implemented in any of the manners and take any of the forms described above for the components of application 150.

Data preprocessing component 252 is configured to perform preprocessing (e.g., normalization) on data reflecting activity of a plurality of genes obtained from a test sample.

Activity level determination component 254 is configured to determine an activity of a plurality of genes in a test sample of the patient.

Expression profile construction component 256 is configured to construct an expression profile by processing the data reflecting activity of a plurality of genes.

Dysregulation scoring component 258 is configured to process an expression profile to calculate scores proportional to a degree of dysregulation in a given subnetwork module.

Risk evaluation component 260 is configured to process a clinical indicator of the patient to determine a risk associated with the disease. Risk evaluation component 260 may use a model for predicting patient outcomes for patients having a disease, the model trained with a plurality of reference dysregulation scores and a plurality of reference clinical indicators. A trained model may be constructed at device 20 in the manners described herein for model construction component 160. A trained model may also be received at device 20 from device 10.

These components of application 250 (or a subset thereof) may cooperate to implement methods detailed herein.

In particular, they may implement a method of prognosing or classifying a patient using a biomarker comprising a plurality of subnetwork modules. The method including: determining (e.g., by activity level determination component 254), an activity of a plurality of genes in a test sample of the patient, said plurality of genes associated with the plurality of subnetwork modules; constructing (e.g., by expression profile construction component 256) an expression profile using the activity of the plurality of genes; determining (e.g., by dysregulation scoring component 258), dysregulation of each of the plurality of subnetwork modules by calculating a score proportional to a degree of dysregulation in each of the plurality of subnetwork modules from said expression profile; prognosing or classifying (e.g., by risk evaluation component 260) the patient by: inputting each dysregulation score into a model for predicting patient outcomes for patients having a disease, the model trained with a plurality of reference dysregulation scores and a plurality of reference clinical indicators; and inputting a clinical indicator of the patient into the model to obtain a risk associated with the disease.

The method may also include normalizing the activity of the plurality of genes using at least one control by, for example, data preprocessing component 252, in substantially the same manner as data preprocessing component 152, described above.

A risk associated with the disease may refer to the probability or expected probability of a disease occurring or reoccurring in a given patient. This, for example in the context of cancer, may be expressed as distant recurrence free survival or distant metastasis free survival (DRFS), or the length of time after primary treatment ends for a cancer that the patient survives without any signs or symptoms of that cancer, or before death of that patient for any cause. Examples of primary cancer treatments include, but are not limited to, endocrine therapy, chemotherapy, radiotherapy, hormone therapy, surgery, gene therapy, thermal therapy, and ultrasound therapy. However, risk may be associated with diseases other than cancer, and therefore other metrics of risk may be used. For example, risk may be expressed as overall survival (OS), which represents the length of time from either the date of diagnosis or the start of treatment for a disease that patients diagnosed with the disease are still alive.

Alternatively, the risk associated with the disease may be expressed as either a low, medium, and/or high risk of disease relapse, and for example, may correspond to a standard or commonly used risk scoring system, for example the Oncotype DX risk score in respect of cancer. For example, if risk is expressed as either a high or low risk, an Oncotype DX score of under 24.5 for a patient may be designated as low risk for relapse, while a patient's score greater than 24.5 may be designated as high risk for relapse. Low or high risk thresholds may also be modified in accordance with any other standard disease relapse risk scoring system in order to accommodate specific risks associated with any one disease. For example, the risk may also correspond with specific values associated with the MammaPrint gene signature risk scoring system.

Clinical indicators may be any measured or observed pathological or clinical metric of a patient, a patient's tumour, or a metric relating to a molecular marker associated with the patient. Clinical indicators may, in respect of cancer for example, comprise the TNM Classification of Malignant Tumours (TNM), wherein the size and growth of a tumour (T), whether cancer has spread to lymph nodes (N) and whether cancer has spread to different parts of the body (M), is determined and scored. Each of or all of these indicators may be relevant as part of a biomarker. Other cancers may have their own classification systems, or may have different relevant metrics. For example, prostate cancer may be scored using a Gleason score, while lymphoma may be staged using the Ann Arbor staging system. Additional clinical indicators may, for example, be tumour size, tumour location, cancerous cell type (for example, squamous cell or adenocarcinoma in the case of esophageal cancers), or may be levels of a specific molecule (i.e., prostate specific antigen in respect of prostate cancer) measured in, for example, the blood or serum of a patient.

The components of application 250 (or a subset thereof) may also cooperate to implement a method of prognosing or classifying a patient comprising: determining (e.g., by activity level determination component 254) mRNA abundance using a sample of a breast cancer tumour of the patient for the group of genes comprising: GSK3B, AKT1S1, RHEB, TSC1, TSC2, RPS6KB1, RPTOR, MTOR, RICTOR, ERBB2, MKI67, ESR1 and PGR, each of said genes associated with at least one node of the PIK3 cell signalling pathway; constructing (e.g., by expression profile construction component 256) an expression profile from the normalized mRNA abundance; comparing (e.g., by risk evaluation component 260) said expression profile to a plurality of reference expression profiles and comparing clinical indicators of the patient to a plurality of reference clinical indicators, wherein the clinical indicators comprise N-stage and tumour size, and wherein each of the plurality of reference expression profiles and each of the reference clinical indicators are associated with a predetermined residual risk of breast cancer; and selecting the reference expression profile most similar to the expression profile and the reference clinical indicators most similar to the patient clinical indicators, to obtain a residual risk associated with breast cancer.

The method may also include normalizing the activity of the plurality of genes using at least one control by, for example, data preprocessing component 252, in substantially the same manner as data preprocessing component 152, described above.

As used herein, “residual risk” refers to the probability or risk of cancer recurrence in breast cancer patients after primary treatment. Residual risk may, for example, be expressed as distant recurrence free survival or distant metastasis free survival (DRFS), or the length of time in, for example, days, months or years, after primary treatment ends for a cancer that the patient survives without any signs or symptoms of that cancer or before death of that patient for any cause. Examples of primary cancer treatments include, but are not limited to, endocrine therapy, chemotherapy, radiotherapy, hormone therapy, surgery, gene therapy, thermal therapy, and ultrasound therapy.

Referring again to FIG. 1, as noted, patient prognosis/classification device 10 and biomarker/pathway identification device 20 may be interconnected by a network 30. Network 30 may be any network capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

### Breast Cancer Prognostic Biomarker: Examples

Biomarker construction/pathway identification device 10 and patient prognosis/classification device 20 are further described with reference to constructing and using an example biomarker for breast cancer. For this example biomarker, each subnetwork module corresponds to a node of a signaling pathway, namely the PIK3CA pathway.

First, biomarker/pathway identification device 10 is configured and operated to construct the breast cancer biomarker. Then, patient prognosis/classification device 20 is configured and operated to use the breast cancer biomarker to perform patient prognosis and classification.

### Materials & Methods

### Study Population

The TEAM trial is a multinational, randomised, open-label, phase III trial in which postmenopausal women with hormone receptor-positive luminal [20] early breast cancer were randomly assigned to receive exemestane (25 mg), once daily or tamoxifen (20 mg) once daily for the first 2.5-3 years followed by exemestane (total of 5 years treatment). This study complied with the Declaration of Helsinki, individual ethics committee guidelines, and the International Conference on Harmonisation and Good Clinical Practice guidelines; all patients provided informed consent. Distant metastasis free survival (DRFS) was defined as time from randomisation to distant relapse or death from breast cancer [20].

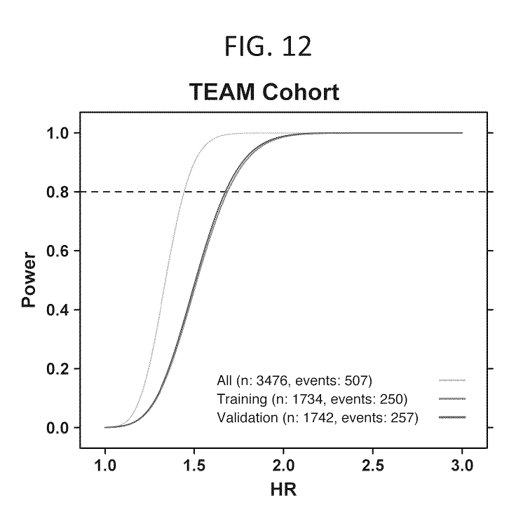

The TEAM trial included a well-powered pathology research study of over 4,500 patients from five countries (FIG. 12). Power analysis was performed to confirm the study size is adequate to detect a HR of at least 3. After mRNA extraction and Nanostring analysis 3,476 samples were available. Patients were randomly assigned to either a training cohort (n=1,734) or the validation cohort (n=1,742) by randomly splitting the 297 NanoString nCounter cartridges into two groups. The training and validation cohorts are statistically indistinguishable from one another and from the overall trial cohort (Table 1) [21, 22].

TABLE 1
Patient demographics: Distribution of patients' tumour and clinical
characteristics in randomly assigned Training and Validation cohorts.
Numbers in the parentheses indicate relative proportion within each
group. Unequal distribution of patient characteristics across randomly
assigned Training and Validation cohorts was tested using Fisher's exact
test followed by adjustment for multiple comparisons (Benjamini &
Hochberg). Patients within the pathology research study were well
matched to the overall TEAM trial cohort see Bartlett et al. (Benjamini
Y, Hochberg Y. Controlling the false discovery rate: a practical and
powerful approach to multiple testing. J Roy Statist Soc Ser B
(Methodological) 1995; 57:289-300 and Bartlett JMS, Brookes CL,
Robson T et al. Estrogen Receptor and Progesterone Receptor As
Predictive Biomarkers of Response to Endocrine Therapy: A
Prospectively Powered Pathology Study in the Tamoxifen and
Exemestane Adjuvant Multinational Trial. Journal of Clinical Oncology
2011;29(12):1531-1538).

| | | | | | P |
|---|---|---|---|---|---|
| | | | Training | Validation | (Training vs. |
| | | Overall | Cohort | Cohort | Validation) |
| Samples | | 3476 | 1734 | 1742 | |
| Age | | | | | 0.88 |
| ≧55 | | 3020 (87%) | 1505 (87%) | 1515 (87%) | |
| <55 | | 455 (13%) | 229 (13%) | 226 (13%) | |
| Grade | | | | | 0.18 |
| 1 | | 351 (11%) | 159 (10%) | 192 (12%) | |
| 2 | | 1769 (53%) | 913 (55%) | 856 (52%) | |
| 3 | | 1196 (36% | 586 (35%) | 610 (37%) | |
| Number of | | | | | 0.88 |
| positive nodes | | | | | |
| 0 | | 1334 (39%) | 669 (40%) | 665 (39%) | |
| 1-3 | | 1493 (44%) | 731 (43%) | 762 (45%) | |
| 4-9 | | 389 (11%) | 196 (12%) | 193 (11%) | |
| 10+ | | 182 (5%) | 96 (6%) | 86 (5%) | |
| Tumour Size | | | | | 0.25 |
| ≦2 | cm | 1593 (46%) | 770 (44%) | 823 (47%) | |
| >2 ≦ 5 | cm | 1671 (48%) | 847 (49%) | 824 (47%) | |
| >5 | cm | 212 (6%) | 117 (7%) | 95 (5%) | |

| | | | | |
|---|---|---|---|---|
| HER2 | | | | 0.18 |
| Negative | 2907 (87%) | 1427 (85%) | 1480 (88%) | |
| Positive | 451 (13%) | 244 (15%) | 207 (12%) | |

At device 10, datastore 144 was populated with patient records created for patients of the TEAM trial cohort.

### RNA Extraction

Five 4 μm formalin-fixed paraffin-embedded (FFPE) sections per case were deparaffinised, tumor areas were macro-dissected and RNA extracted according to Ambion® Recoverall™ Total Nucleic Acid Isolation Kit-RNA extraction protocol (Life Technologies™, Ontario, Canada) except for one change: samples were incubated in protease for 3 hours instead of 15 minutes. RNA samples were eluted and quantified using a Nanodrop-8000 spectrophometer (Delaware, USA). Samples, where necessary, underwent sodium-acetate/ethanol re-precipitation. RNAs extracted from 3,476 samples were successfully analysed.

mRNA Abundance Analysis

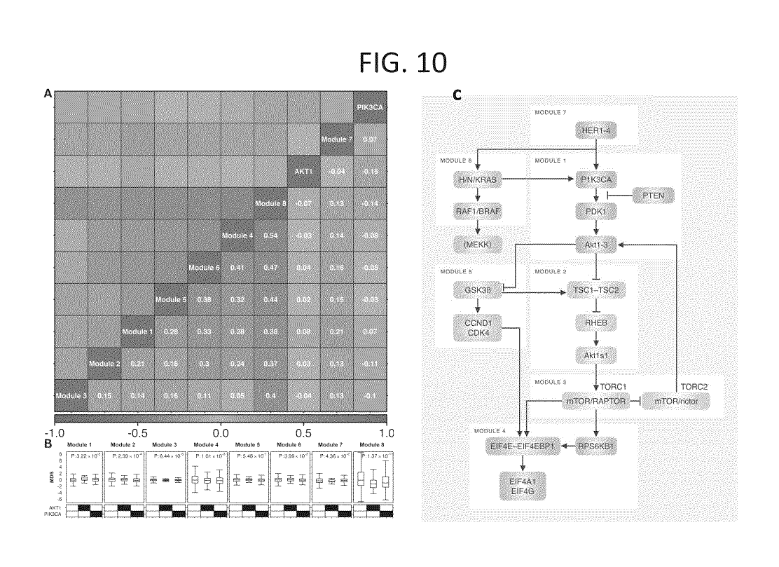

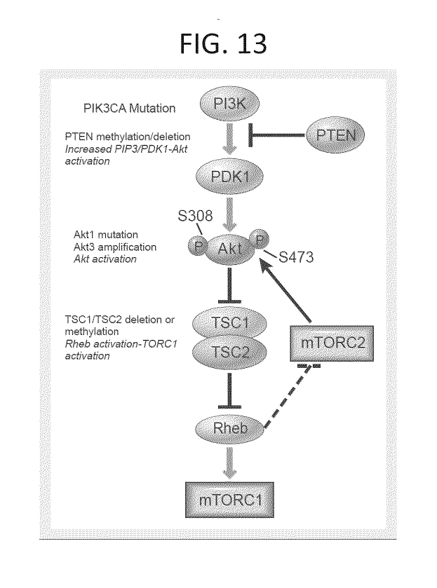

Thirty-three genes of interest were selected from the PIK3CA signalling pathway and 6 reference genes. Genes of interest were selected specifically to interrogate key functional nodes within the PIK3CA signalling pathway [24, 25] as shown in FIG. 10C, FIG. 13 and Table 2.

TABLE 2
PIK3CA pathway modules: List of PIK3CA pathway modules and
corresponding genes. Modules were derived on the basis of underlying
biological functionality.

| | Module Name | Genes |
|---|---|---|
| Module 1 | PIK3CA/AKT | AKT1, AKT2, AKT3, PDK1, PIK3CA, |
| | signalling | PTEN |
| Module 2 | Rheb activation | GSK3B, AKT1S1, TSC1, TSC2, RHEB |
| Module 3 | mTOR signalling | RPS6KB1, RAPTOR, RICTOR, mTOR |
| Module 4 | Protein translation | EIF4EBP1, EIF4G1, GSK3B, EIF4E, |
| | | EIF4A1, RPS6KB1 |
| Module 5 | GSK3B signalling | GSK3B, CDK4, CCND1 |
| Module 6 | RAS | KRAS, HRAS, NRAS, RAF1, BRAF |
| Module 7 | ERBB | ERBB2, EGFR, ERBB3, ERBB4 |
| Module 8 | IHC4 biomarker | MKI67, ERBB2, ESR1, PGR |

Probes for each gene were designed and synthesised at NanoString® Technologies (Washington, USA). RNA samples (400 ng; 5 μL of 80 ng/4) were hybridised, processed and analysed using the NanoString® nCounter® Analysis System, according to NanoString® Technologies protocols.

### Data Pre-Processing

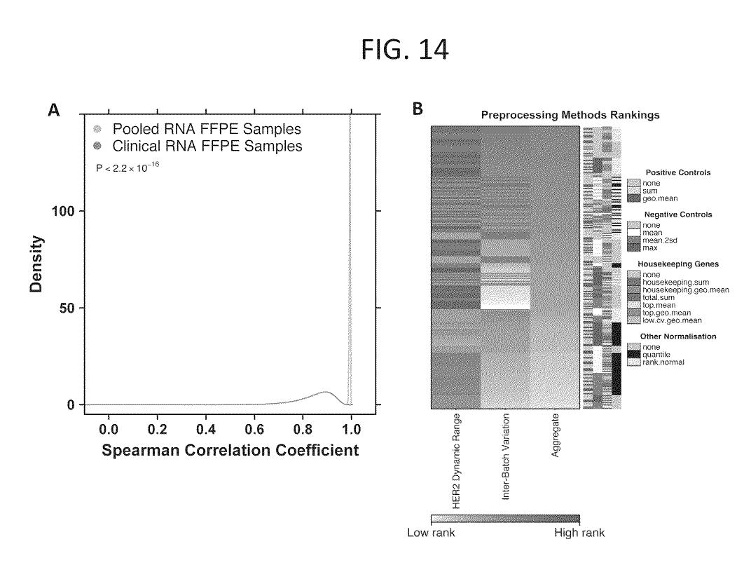

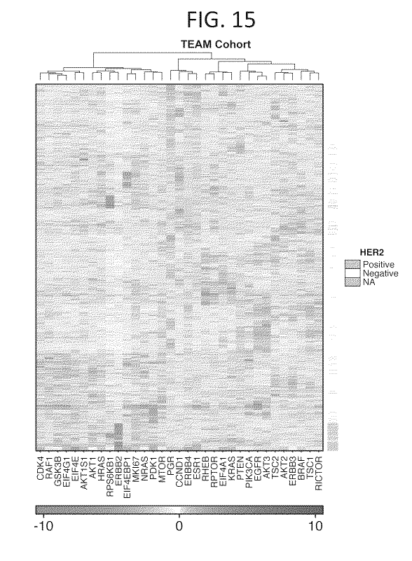

At device 10, raw mRNA abundance counts data were pre-processed by data preprocessing component 152, which incorporated the R package NanoStringNorm [26] (v1.1.16), as further detailed below. A range of pre-processing schemes was assessed to identify the most optimal normalisation parameters. (FIGS. 14 and 15).

### Survival Modelling

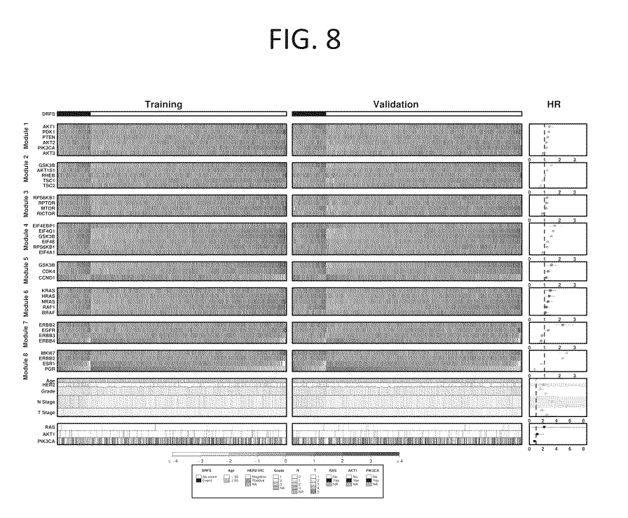

Univariate survival analysis of processed mRNA abundance data was performed by median-dichotomizing patients into high- and low-risk groups, except for ERBB2 (FIG. 8; Table 3) where risk groups were determined via expectation-maximization clustering (k=2) because of the existence of two discrete populations of ERBB2 expressing cancers and the small proportion (<15%) of HER2/ERBB2 positive tumors [27, 28]. Survival analysis of clinical variables was performed by modelling age as binary variable (dichotomized at age 55), while grade, nodal status and tumor size were modelled as ordinal variables (Table 4). For mRNA and IHC4 models, tumor size was treated as a continuous variable. Univariate survival analysis of mutational profiles (AKT1, PIK3CA and RAS [12]; Table 4) was performed by dichotomizing patients into mutant and wild-type groups.

TABLE 3
Univariate Gene-Wise Analyses: Univariate prognostic assessment of mRNA abundance profiles.
For both TEAM Training and Validation cohorts, patients were median-dichotomized into low-
and high-risk groups except for ERBB2 (HER2). ERBB2 dichotomization was performed using
Expectation-maximization clustering. DRFS was used as the survival end point. Cox proportional
hazards model was used to estimate the Hazard ratios followed by the Wald-test for the significance
of difference between the risk groups. P values were corrected for multiple comparisons
using Benjamini & Hochberg method. The varying n within Training and Validation cohorts
is an artefact of rank normalisation resulting in NA for some patients.

| | Training Cohort | | | | Validation Cohort | | | |
|---|---|---|---|---|---|---|---|---|
| | | | Wald | | | | Wald | |
| Gene | HR | 95% CI | P_{adjusted} | N | HR | 95% CI | P_{adjusted} | N |
| PgR | 0.347 | 0.263-0.459 | 2.82 × 10⁻¹² | 1734 | 0.441 | 0.338-0.575 | 2.42 × 10⁻⁸ | 1740 |
| Ki67 | 2.472 | 1.888-3.238 | 8.31 × 10⁻¹⁰ | 1733 | 2.837 | 2.197-3.664 | 4.53 × 10⁻¹⁴ | 1740 |
| HER2 | 2.208 | 1.646-2.961 | 1.44 × 10⁻⁶ | 1734 | 1.82 | 1.323-2.504 | 0.000882857 | 1741 |
| 4EBP1 | 1.673 | 1.297-2.158 | 0.000627917 | 1734 | 1.957 | 1.526-2.509 | 1.35 × 10⁻⁶ | 1742 |
| E1F4G | 1.57 | 1.218-2.024 | 0.003385337 | 1734 | 1.61 | 1.26-2.057 | 0.000669264 | 1741 |
| GSK3B | 1.462 | 1.137-1.88 | 0.017501496 | 1734 | 1.751 | 1.371-2.238 | 5.05 × 10⁻⁵ | 1741 |
| KRAS | 1.391 | 1.082-1.788 | 0.048135757 | 1734 | 1.554 | 1.216-1.986 | 0.001444643 | 1742 |
| TSC2 | 0.733 | 0.57-0.942 | 0.064128252 | 1734 | 0.817 | 0.636-1.05 | 0.176433949 | 1741 |
| AKT1 | 1.326 | 1.033-1.703 | 0.101980935 | 1734 | 1.462 | 1.144-1.868 | 0.006199282 | 1742 |
| HRAS | 1.317 | 1.026-1.69 | 0.105060417 | 1733 | 1.802 | 1.41-2.303 | 2.18 × 10⁻⁵ | 1741 |
| HER4 | 0.775 | 0.604-0.995 | 0.128940064 | 1732 | 0.622 | 0.484-0.799 | 0.000868759 | 1742 |
| PDK1 | 1.295 | 1.009-1.662 | 0.128940064 | 1734 | 1.636 | 1.281-2.09 | 0.00045264 | 1741 |
| ERa | 0.797 | 0.621-1.023 | 0.187982965 | 1734 | 0.958 | 0.749-1.225 | 0.753696978 | 1741 |
| HER1 | 1.252 | 0.976-1.607 | 0.187982965 | 1734 | 0.817 | 0.637-1.048 | 0.176433949 | 1740 |
| CDK4 | 1.238 | 0.965-1.589 | 0.201385334 | 1731 | 1.102 | 0.858-1.415 | 0.525586912 | 1742 |
| NRAS | 1.236 | 0.964-1.586 | 0.201385334 | 1734 | 1.272 | 0.992-1.63 | 0.09829097 | 1742 |
| PTEN | 1.216 | 0.948-1.559 | 0.248438794 | 1734 | 1.136 | 0.887-1.455 | 0.392313002 | 1742 |
| E1F4E | 1.205 | 0.939-1.545 | 0.267517742 | 1734 | 1.444 | 1.127-1.849 | 0.008931455 | 1742 |
| HER3 | 0.833 | 0.649-1.068 | 0.267517742 | 1734 | 0.92 | 0.716-1.181 | 0.580481046 | 1741 |
| PRAS40 | 1.185 | 0.924-1.519 | 0.308813806 | 1734 | 0.926 | 0.717-1.195 | 0.6074361 | 1741 |
| p70S6K | 1.166 | 0.909-1.495 | 0.366803317 | 1734 | 1.271 | 0.993-1.628 | 0.09829097 | 1741 |
| RICTOR | 0.866 | 0.675-1.11 | 0.393871202 | 1733 | 0.749 | 0.581-0.967 | 0.052496355 | 1740 |
| RAPTOR | 1.14 | 0.889-1.461 | 0.446892152 | 1734 | 1.176 | 0.92-1.502 | 0.276433869 | 1741 |
| AKT2 | 1.122 | 0.875-1.438 | 0.449568658 | 1734 | 1.021 | 0.795-1.31 | 0.873231577 | 1742 |
| AKT3 | 0.898 | 0.701-1.151 | 0.449568658 | 1734 | 0.823 | 0.642-1.055 | 0.182793196 | 1742 |
| CCND1 | 1.115 | 0.87-1.429 | 0.449568658 | 1734 | 1.362 | 1.066-1.74 | 0.028490089 | 1741 |
| E1F4A | 0.895 | 0.698-1.147 | 0.449568658 | 1734 | 1.142 | 0.892-1.462 | 0.381943628 | 1742 |
| PI3KCA | 1.12 | 0.874-1.436 | 0.449568658 | 1734 | 1.498 | 1.172-1.915 | 0.003704662 | 1742 |
| RAF1 | 1.123 | 0.876-1.44 | 0.449568658 | 1733 | 1.389 | 1.085-1.777 | 0.02075063 | 1742 |
| TSC1 | 0.883 | 0.688-1.131 | 0.449568658 | 1733 | 0.774 | 0.598-1.002 | 0.097049395 | 1740 |
| mTOR | 1.1 | 0.858-1.409 | 0.497211439 | 1734 | 1.069 | 0.828-1.38 | 0.647254297 | 1742 |
| BRAF | 1.056 | 0.824-1.354 | 0.70666752 | 1734 | 0.895 | 0.691-1.158 | 0.483448043 | 1741 |
| RHEB | 1.025 | 0.8-1.314 | 0.870767566 | 1733 | 1.497 | 1.171-1.915 | 0.003704662 | 1741 |
| RHEB/ | 0.986 | 0.77-1.264 | 0.913378512 | 1734 | 0.862 | 0.665-1.117 | 0.353719924 | 1741 |
| RHEBP1 | | | | | | | | |

TABLE 4
Univariate prognostic assessment of clinical variables and mutational profiles. DRFS was
used as the survival end point. Cox proportional hazards model was used to estimate the
Hazard ratios. The significance of association between DRFS and dichotomous variables (Age,
HER2 Status, and mutational profiles) was assessed using the Wald-test. However, Log-rank
test was used for multi-category variables (grade, T-stage and N-stage). Prognostic assessment
of grade and stage was conducted such that the grade 2 and 3 patients were compared against
the baseline grade 1; N Stage 1, 2 and 3 were compared against N Stage 0 (node-negative);
and T Stage 2 and 3 were compared against the baseline T Stage 1.

| | Training | | | | Validation | | | |
|---|---|---|---|---|---|---|---|---|
| Variable | HR | 95% CI | P value | N | HR | 95% CI | P value | N |
| Age | 0.964 | 0.67-1.38 | 0.84 | 1734 | 1.190 | 0.81-1.74 | 0.37 | 1741 |
| Grade | | | | | | | | |
| 1 vs 2 | 1.583 | 0.89-2.80 | 0.84 | 1658 | 2.537 | 1.37-4.70 | 0.003 | 1658 |
| 1 vs 3 | 2.450 | 1.38-4.35 | 0.002 | | 3.499 | 1.88-6.50 | 7.28 × 10⁻⁵ | |
| Nodal status | | | | | | | | |
| 0 vs 1-3 | 1.183 | 0.86-1.63 | 0.31 | 1692 | 1.422 | 1.04-1.94 | 0.026 | 1706 |
| 0 vs 4-9 | 3.377 | 2.36-4.82 | 2.19 × 10⁻¹¹ | | 3.050 | 2.11-4.40 | 2.55 × 10⁻⁹ | |
| 0 vs 10+? | 5.604 | 3.79-8.28 | 0? | | 5.422 | 3.56-8.25 | 2.89 × 10⁻¹⁵ | |
| Tumour | | | | | | | | |
| Size | | | | | | | | |
| <2 vs ≧2 | 1.86 | 1.41-2.46 | 1.02 × 10⁻⁵ | 1731 | 1.601 | 1.23-2.09 | 0.0005 | 1738 |
| <2 vs ≧5 | 2.64 | 1.70-4.09 | 1.47 × 10⁻⁵ | | 3.174 | 2.08-4.85 | 9.2 × 10⁻⁸ | |
| HER2 | 2.104 | 1.57-2.82 | 7.45 × 10⁻⁷ | 1671 | 1.486 | 1.06-2.09 | 0.02 | 1738 |
| PIK3CA | 0.750 | 0.57-0.98 | 0.08 | 1670 | 0.814 | 0.63-1.05 | 0.19 | 1674 |
| AKT1 | 1.165 | 0.62-2.19 | 0.64 | 1670 | 0.892 | 0.42-1.89 | 0.76 | 1674 |
| RAS | 2.191 | 0.31-15.6 | 0.43 | 1670 | 0.617 | 0.09-4.40 | 0.63 | 1674 |

### IHC4 Model

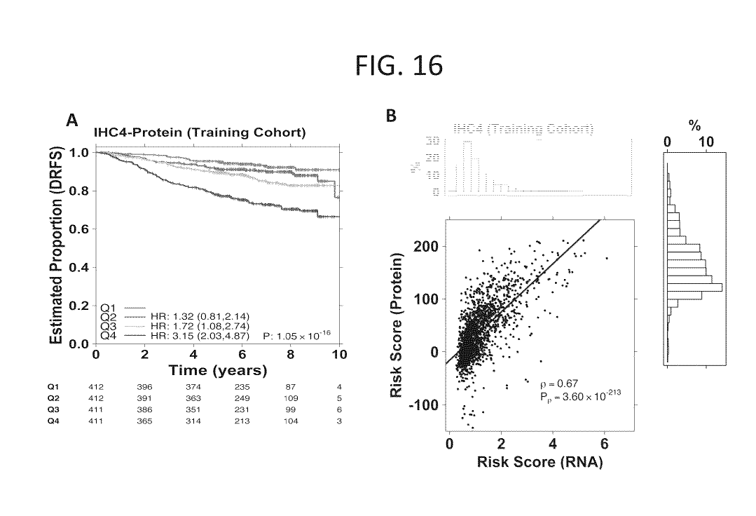

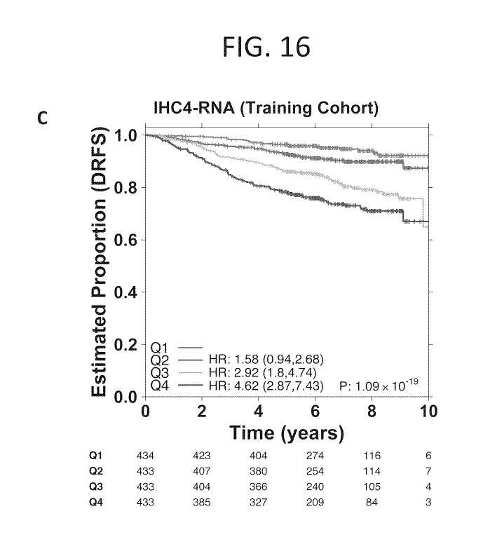

IHC4-protein model risk scores were calculated as described by Cuzick et al. and further adjusted for clinical covariates. An IHC4-mRNA model was trained on mRNA abundance profiles of ESR1, PGR, ERBB2 and MKI67 in the training cohort using multivariate Cox proportional hazards modelling (Table 5). Model predictions (continuous risk scores) were grouped into quartiles (FIG. 16) and analysed using Kaplan-Meier analysis and multivariate Cox proportional hazards model adjusted for clinical variables as above.

TABLE 5
Multivariate prognostic model using mRNA abundance profiles
(TEAM Training cohort) of IHC4 marker genes; ESR1, PGR, ERBB2
and MKI67. Model parameters were estimated using Cox proportional
hazards model, and subsequently used to predict patient risk score
(risk.score) in the TEAM Training and Validation cohorts. Survival
differences between the median-dichotomized risk scores (risk.group)
as well as quartiles (risk.group.quartiles) of the risk score were
assessed using Kaplan-Meier analysis.

| | coef | exp(coef) | se(coef) | z | Pr(>\|z\|) |
|---|---|---|---|---|---|
| ESR1 | −0.008204 | 0.991829 | 0.053632 | −0.153 | 0.87842 |
| PGR | −0.303747 | 0.738047 | 0.069218 | −4.388 | 1.14 × 10⁻⁵ |
| ERBB2 | 0.156425 | 1.169324 | 0.053275 | 2.936 | 0.00332 |
| MKI67 | 0.297402 | 1.346357 | 0.0729 | 4.08 | 4.51 × 10⁻⁵ |

mRNA Network Analysis

The 33 genes were derived from 8 functionally-related modules (FIGS. 8, 9C, 10C and 13).

Datastore 144 was populated with subnetwork records created for each of these 8 modules.

At device 10, for each functional module, module scoring component 154 calculated a ‘module-dysregulation score’ (MDS). Module-specific MDSs were subsequently used in multivariate Cox proportional hazards modelling by model construction component 160, adjusted for clinical covariates as above. All models were trained in the training cohort and validated in the fully-independent validation cohort (Table 1) using DRFS truncated to 10 years as an end-point. Recurrence probabilities were estimated as described below. All survival modelling was performed on distant metastasis free survival (DRFS), in the R statistical environment with the survival package (v2.37-4) and model performance compared through area under the receiver operating characteristic (ROC) curve (see below).

### TEAM Cohort Power Calculations

Power calculations were performed on complete TEAM cohort (n=3,476; events=507) and for each of the training (n=1,734; events=250) and validation (n=1,742; events=257) subsets separately. Power estimates representing the likelihood of observing a specific HR against the above-mentioned events, (assuming equal-sized patient groups) were derived using the following formula [41]:

$$\begin{matrix}{z}_{power} = \frac{\sqrt{E} \times ln ( HR )}{2} - z ( 1 - \frac{α}{2} )\end{matrix} \qquad ( 1 )$$

where E represents the total number of events (DRFS) and a represents the significance level which was set to 10⁻³. z_{power} was calculated for HR ranging from 1 to 3 with steps of 0.01.

mRNA Abundance Data Processing

As noted, raw mRNA abundance counts data were preprocessed by data preprocessing component 152 incorporating the R package NanoStringNorm [15] (v1.1.16). In total, 252 preprocessing schemes were evaluated; spanning normalization with respect to six positive controls, eight negative controls and six housekeeping genes (GUSB, PUM1, SF3A1, TBP, TFRC and TMED10) followed by global normalization (FIGS. 14 and 15). To identify the optimal preprocessing parameters, two criteria were defined. First, each of the 252 preprocessing schemes was ranked based on their ability to maximize Euclidean distance of ERBB2 mRNA abundance between HER2-positive and HER2-negative samples. The process was repeated for 1000 random subsets of HER2-positive and HER2-negative samples for each of the preprocessing schemes. Second, using 37 replicates of an RNA pool extracted from 4 randomly selected anonymized FFPE breast tumor samples, preprocessing schemes were ranked based on inter-batch variation. To this end, mixed effects linear models were used and residual estimates were used as a measure of inter-batch variation (R package: nlme v3.1-113). Cumulative ranks based on these two criteria were estimated using RankProduct [16] resulting in selection of an optimal pre-processing scheme of normalisation to the geometric mean derived from all genes followed by rank normalisation (FIG. 15). Samples with RNA content |z-score|>6 were discarded as being potential outliers. Only one sample was removed from the top preprocessing scheme. Six samples were run in duplicates, and their raw counts were averaged and subsequently treated as a single sample. Training and validation cohorts were created by randomly splitting 297 NanoString nCounter cartridges into two groups (Table 1), which ensures that there are no batch-effects shared between the two cohorts.

Patient records in datastore 144 were updated to reflect the data, as preprocessed by data processing component 152.

As will be appreciated, in some embodiments, raw measurements may be used to calculate MDS, and preprocessing may be avoided.

### Module Dysregulation Score

At device 10, predefined functional modules reflected in the subnetwork records in datastore 144 were scored by module scoring component 154 using a two-step process. First, weights (β) of all the genes were estimated by fitting a univariate Cox proportional hazards model (Training cohort only). Second, these weights were applied to scaled mRNA abundance profiles to estimate per-patient module dysregulation score using the following equation:

$$\begin{matrix}MDS = {\sum }_{i = 1}^{n} β {X}_{i}\end{matrix} \qquad ( 2 )$$

where n represents the number of genes in a given module and Xᵢ is the scaled (z-score) abundance of gene i. MDS was subsequently used in the multivariate Cox proportional hazards model alongside clinical covariates.

### Survival Modelling

Univariate survival analysis of mRNA abundance data was performed by median-dichotomizing patients into high- and low-risk groups, except for ERBB2 (Table 3). ERBB2 risk groups were determined with expectation-maximization clustering (k=2) using R package mclust (v4.2). Univariate survival analysis of clinical variables was performed by modelling age as binary variable (dichotomized at age≧55), while grade, N-stage and T-stage were modelled as ordinal variables (Table 4). Univariate survival analysis of mutational profiles (AKT1, PIK3CA and RAS; Table 4) was performed by dichotomizing patients into mutant and wild-type groups.

At device 10, MDS profiles (equation 2) of patients in the Training cohort were used to fit a multivariate Cox proportional hazards model alongside clinical variables by processing the patient records and subnetwork records in datastore 144. Through a backwards step-wise refinement algorithm implemented in module selection component 158 following ranking of the modules by module ranking component 156, a module-based risk model containing selected subnetwork modules was created by model construction component 160 (Table 7). The parameters estimated by the multivariate model were applied to the MDS and clinical profiles of patients in the Validation cohort to generate per-patient risk score. These risk scores (continuous) were grouped into quartiles using the thresholds derived from the Training cohort, and resulting groups were subsequently evaluated through Kaplan-Meier analysis.

TABLE 7
Multivariate Modules-derived prognostic model. Model parameters
were estimated using a multivariate Cox proportional hazards model
initialized with eight mRNA modules (FIG. 1), age, grade,
pathological size and N-stage. Model was further refined using
backwards elimination resulting in the variables presented in the first
table. The refined model was subsequently used to predict patient
risk score (risk.score) in the TEAM Training and Validation cohorts.
Survival differences between the median-dichotomized risk scores
(risk.group) as well as quartiles (risk.group.quartiles) of the risk
scores were assessed using Kaplan-Meier analysis.

| analysis. | coef | exp(coef) | se(coef) | z | Pr(>\|z\|) |
|---|---|---|---|---|---|
| Module 2 | 0.11349 | 1.12018 | 0.08892 | 1.276 | 2.02 10⁻¹ |
| Module 3 | −0.25609 | 0.77407 | 0.17452 | −1.467 | 0.14228 |
| Module 7 | −0.09618 | 0.9083 | 0.05698 | −1.688 | 9.14 × 10⁻² |
| Module 8 | 0.20169 | 1.22346 | 0.03316 | 6.083 | 1.18 × 10⁻⁹ |
| N Stage-1 | 0.32735 | 1.38729 | 0.16815 | 1.947 | 5.16 × 10⁻² |
| N Stage-2 | 1.24807 | 3.48361 | 0.18991 | 6.572 | 4.97 × 10⁻¹¹ |
| N Stage-3 | 1.41443 | 4.11412 | 0.21555 | 6.562 | 5.31 × 10⁻¹¹ |
| Pathological | 0.14558 | 1.15671 | 0.04274 | 3.406 | 0.00066 |
| Size | | | | | |

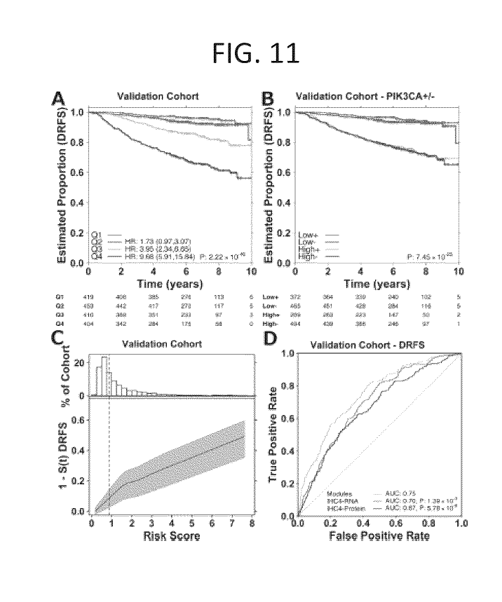

At device 20, the biomarker comprising the selected subnetwork modules may be used by patient prognosis/classification application to perform patient prognosis/classification. In particular, application 250 may use the model generated by model construction component 160 to predict patient outcomes. For example, for a given patient with mRNA abundance profile of genes underlying modules in Table 7, MDS can be calculated (equation 2) by dysregulation scoring component 258, then a risk score estimate can be generated by risk evaluation component 260 from the MDS and clinical data to predict the likelihood of relapse using the model in FIG. 11.

More generally, application 250 may implement methods to determine (e.g., by activity level determination component 254), an activity of a plurality of genes in a test sample of the patient, said plurality of genes associated with the plurality of predetermined subnetwork modules. Activity of the genes contained in the biomarker, as described above, may be determined, for example, using mRNA abundance of the genes. mRNA abundance may, for example, be measured using a qPCR or RT-qPCR device which may be interconnected with device 20 by way of an I/O interface 204.

Application 250 may also implement methods to construct (e.g., by expression profile construction component 256) an expression profile of the patient using the determined activity of the plurality of genes. The expression profile may be a data structure, said structure comprising entries, wherein each entry comprises the mRNA abundance data of each of the genes comprising the biomarker for the patient. However, the expression profile may alternatively comprise data corresponding to activity measured, for example, according to one or more of somatic point mutation, small indel, somatic copy-number status, germline copy-number status, somatic genomic rearrangements, germline genomic rearrangements, metabolite abundances, protein abundances and DNA methylation.

The dysregulation of each of the plurality of subnetwork modules for the patient may be calculated by dysregulation scoring component 258 in substantially the same fashion as module scoring component 154, assigning to each of the plurality of subnetwork modules a score proportional to a degree of dysregulation in that subnetwork module based on the patient's expression profile.

Prognosing or classifying the patient may be performed by risk evaluation component 260 implementing the following: inputting each dysregulation score into a model for predicting patient outcomes for patients having a disease, the model trained with a plurality of reference dysregulation scores and a plurality of reference clinical indicators; and inputting a clinical indicator of the patient into the model to obtain a risk associated with the disease, which is described in more detail above.

The IHC4-RNA model was trained on mRNA abundance profiles of ESR1, PGR, ERBB2 and MKI67 in the Training cohort using a multivariate Cox proportional hazards model (Table 5). The model parameters learnt through fitting the multivariate Cox proportional hazards model were subsequently applied to the mRNA abundance profiles of the above-mentioned four genes in the Validation cohort to generate per-patient risk score. These risk scores (continuous) were grouped into quartiles. These groups were evaluated using Kaplan-Meier analysis and multivariate Cox proportional hazards model adjusted for age (binary variable dichotomized at age 55), N-stage (ordinal), tumour size (continuous variable) and grade (ordinal variable). The IHC4-protein model was calculated as described by Cuzick et al [42]. All models were trained and validated using DRFS truncated to 10 years as an end-point.

Recurrence probabilities at 5 years were estimated by binning the predicted risk-scores in 25 equal groups. For each group, recurrence probability R₍ₜ₎ was estimated as 1-S₍ₜ₎, where S₍ₜ₎ is the Kaplan-Meier survival estimate at year 5. The R₍ₜ₎ estimates of 25 groups were smoothed using local polynomial regression fit. The predicted estimates were plotted against the median risk score of each group except the first and last group, where the lowest risk score and 99th percentile were used, respectively. All survival modelling was performed in the R statistical environment (R package: survival v2.37-4).

### Performance Assessment

Performance of survival models was compared through area under the receiver operating characteristic (ROC) curve. Significance of difference between the ROC curves was assessed through permutation analysis (10,000 permutations by shuffling the risk scores while maintaining the order of survival objects). Patients censored before 5 years (Training cohort: n=192, Validation cohort: n=181) were eliminated from sampling. ROC analysis was implemented using R packages pROC (v1.6.0.1) and survivalROC (v1.0.3).

### Visualization

mRNA abundance data shown in the heatmaps (FIG. 8) were scaled to z-scores. Within each module, patients were further sorted by the column sums. Patients with no known information in all clinical covariates were excluded from visualization. In MDS correlation heatmap (FIG. 10A), to circumvent over-estimates between modules sharing genes (GSK3B: Modules 2, 4 and 5; RPS6KB1: 3 and 4; ERBB2: Modules 7 and 8), these genes were removed from the correlation analysis. In FIG. 10B, there was only one patient with double mutant profile, and hence not shown in the figure. Risk score plots were right-truncated at the 99^{th} percentile, however, 5-year recurrence probability of the patients in the right tail of the distribution is shown in the range displayed. Data visualization was performed using lattice (v0.20-24) and latticeExtra (v0.6-26) packages from R statistical environment (v3.0.1 and 3.0.2).

### Results

mRNA abundance profiles of 33 genes were available for 3,476 patients and complete mutational data was available for 3,353 patients [12]. Outcome data were available for 3,343 patients (FIG. 8, Table 1). Patients were randomly divided into a 1,734-patient training cohort (250 events) and a 1,742-patient validation cohort (257 events). Median follow-up [28] in each cohort was 6.7 and 6.8 years respectively.

Univariate mRNA Expression

Tumors from patients who subsequently progressed to metastatic breast cancer showed markedly different mRNA abundance profiles relative to tumors from patients who did not progress during follow up (FIG. 8). Seven genes were univariately prognostic (p_{adjusted}<0.05; PGR, MKI67, ERBB2, EIF4EBP1, EIF4G1, GSK3B and KRAS; Table 3) in the training cohort, of which three are in Module 4 (EIF4EBP1, GSK3B & EIF4G1) and three are in Module 8 (MKI67, ERBB2 & PGR). All seven genes were significantly associated with patient survival in the same direction in the validation cohort. Tumor grade of 3, nodal status, tumor size and HER2 status were univariately prognostic (p<0.01), while PIK3CA mutations were marginally univariately significant [13] (p<0.05; Table 4).

IHC4—mRNA Based Assessment of a Conventional Risk Score

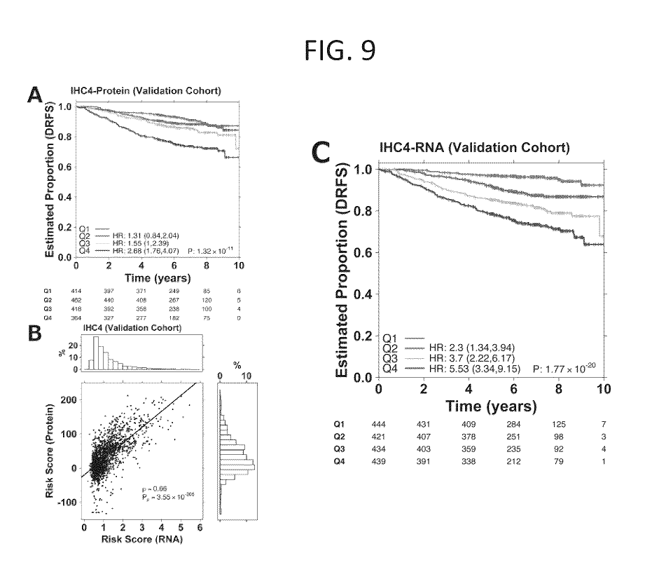

The ability of a protein-based residual risk classifier, IHC4, was evaluated to predict outcome in this large, well-powered cohort (FIG. 12). Using existing data from the TEAM study [29] we determined protein-based IHC4 scores using IHC measurements of ER, PgR, Ki67 and HER2 and tested residual risk prediction following adjustment for age, nodal status, grade and size in both the training (p=1.05×10⁻¹⁶; FIG. 16A) and validation (p=1.32×10⁻¹¹, FIG. 9A) cohorts.

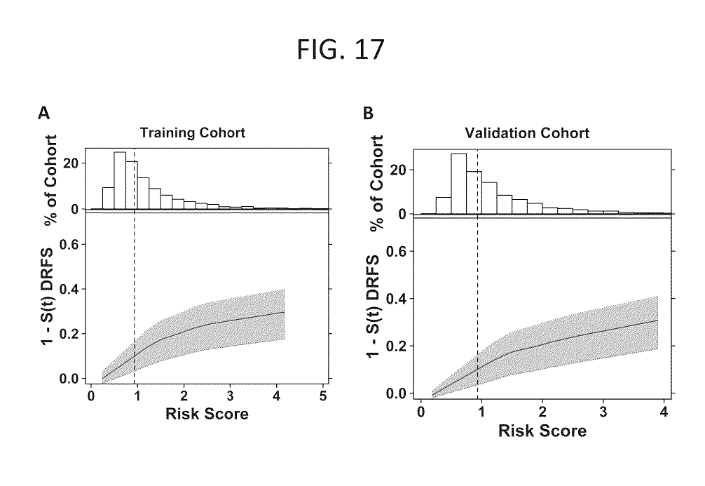

A prognostic model was generated using the mRNA abundances of the IHC4 markers, which we call IHC4-mRNA (Table 5). IHC4-protein and IHC4-mRNA risk scores were well-correlated (p=0.66, p=3.55×10⁻²⁰⁵, FIGS. 9B and 16B), suggesting the mRNA abundance-based classifier can serve as a proxy for the protein-based model. Further, IHC4-mRNA was superior to IHC4-protein in stratifying patients into groups with differential outcome. Comparing the lowest and highest-risk quartiles of patients, IHC4-mRNA provided robust separation (HR=5.53; 95% C1=3.34-9.15; p=1.77×10⁻²⁰, FIGS. 13C, 16C and 17A-B) compared to more modest separation by IHC4-protein (FIG. 9A; HR=2.68; p_{AUC}=0.048, comparing the two models in the validation cohort). These data indicate that IHC4-protein may be substituted by an RNA classifier from the same genes (ESR1, PGR, MKI67 & ERBB2).

### PI3K Signaling Modules Univariately Predict Risk

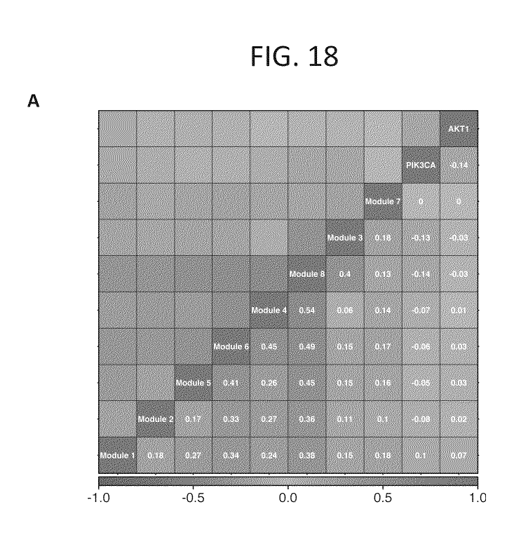

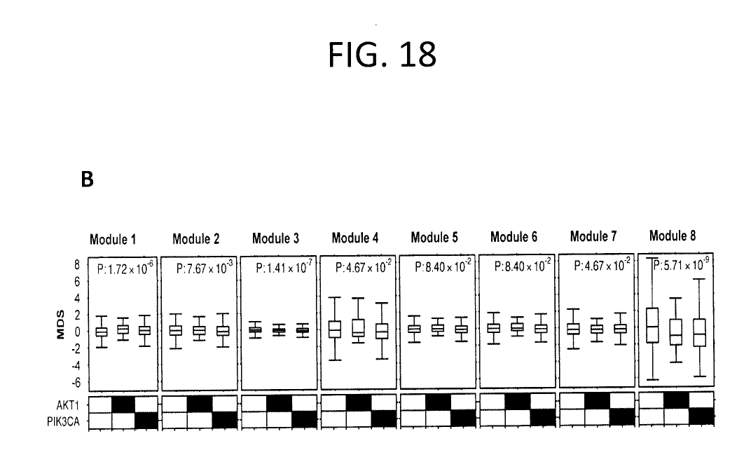

The 33 PI3K pathway genes were aggregated into 8 modules representing different nodes of the pathway. mRNA abundance data within each module was collapsed into a single per patient Module Dysregulation Score (MDS) to enable comparisons between modules and to determine module co-expression. All 8 modules were univariately associated with patient outcome in the training cohort (p<0.05, Table 6). Given that only 7 genes were univariately prognostic (FIG. 8), this provides strong support for the value of pathway-level integration. The independence of these 8 modules was analyzed by calculating the correlations of per-patient MDS for each pair of modules, excluding genes present in multiple modules (FIG. 10A, training cohort; FIG. 18A, validation cohort). Moderate correlations (˜0.45) were observed between somesome module pairs (e.g. Module 8 and Module 4), but most showed weak correlations, suggesting independent prognostic capacity. Finally, per-module dysregulation was compared to the previously determined mutational status of PIK3CA and AKT1 [13]. Modules 1, 2, 3, 4, 6, 7 & 8 showed significant associations with mutation status (one-way ANOVA; p_{adjusted}<0.05; FIGS. 10B and 18B).

TABLE 6
Univariate prognostic assessment of median-dichotomised module-dysregulation
scores (MDS). DRFS was used as the survival end point. Cox proportional
hazards model was used to estimate the Hazard ratios.

| | Training | | | | Validation | | | |
|---|---|---|---|---|---|---|---|---|
| | HR | 95% CI | P value | N | HR | 95% CI | P value | N |
| Module.1 | 1.619 | 1.26-2.09 | 1.95 × 10⁻⁵ | 1734 | 1.759 | 1.37-2.26 | 1.14 × 10⁻⁵ | 1742 |
| Module.2 | 1.735 | 1.34-2.24 | 2.45 × 10⁻⁵ | 1734 | 1.556 | 1.21-2.00 | 5.11 × 10⁻⁴ | 1742 |
| Module.3 | 1.298 | 1.01-1.67 | 0.04 | 1734 | 1.298 | 1.02-1.66 | 0.04 | 1742 |
| Module.4 | 1.991 | 1.53-2.59 | 2.32 × 10⁻⁷ | 1734 | 2.099 | 1.62-2.71 | 1.57 × 10⁻⁸ | 1742 |
| Module.5 | 1.647 | 1.28-2.13 | 1.20 × 10⁻⁴ | 1734 | 1.915 | 1.49-2.47 | 5.63 × 10⁻⁷ | 1742 |
| Module.6 | 1.488 | 1.16-1.91 | 0.002 | 1734 | 2.15 | 1.66-2.79 | 7.83 × 10⁻⁹ | 1742 |
| Module.7 | 1.400 | 1.09-1.80 | 0.009 | 1734 | 1.217 | 0.95-1.56 | 0.18 | 1742 |
| Module.8 | 3.088 | 2.33-4.09 | 4.11 × 10⁻¹⁵ | 1734 | 3.099 | 2.35-4.09 | 1.78 × 10⁻¹⁵ | 1742 |

### Construction of a PIK3CA Signaling Module Residual Risk Signature

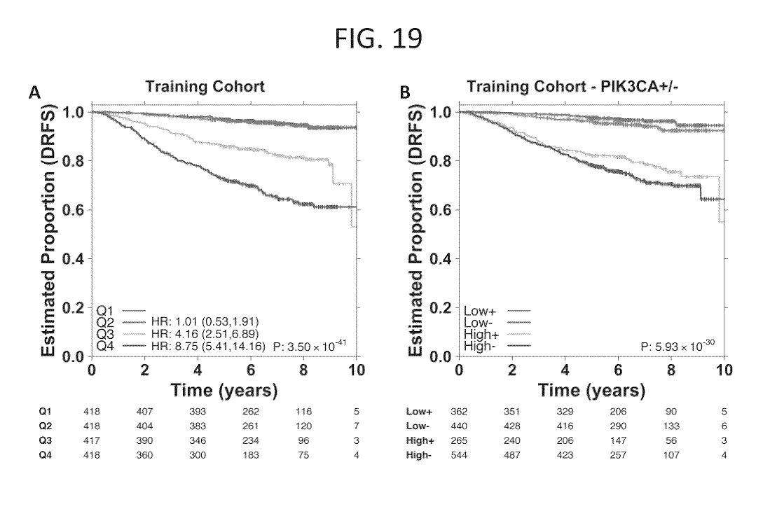

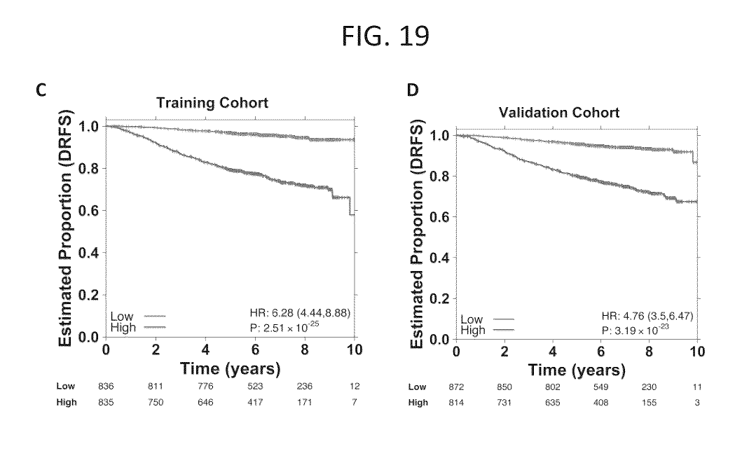

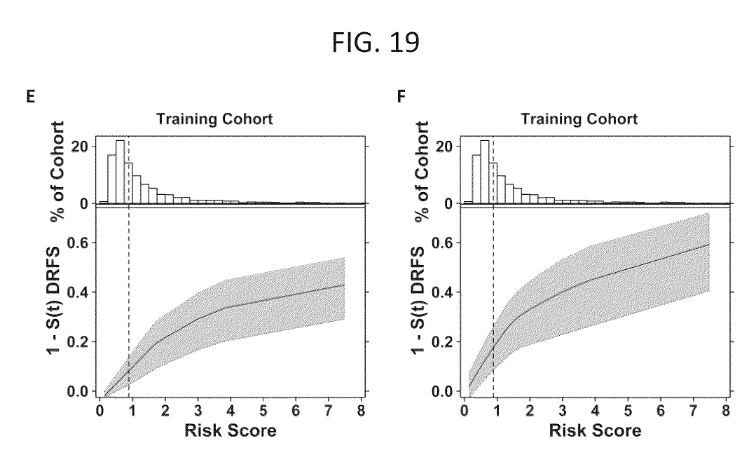

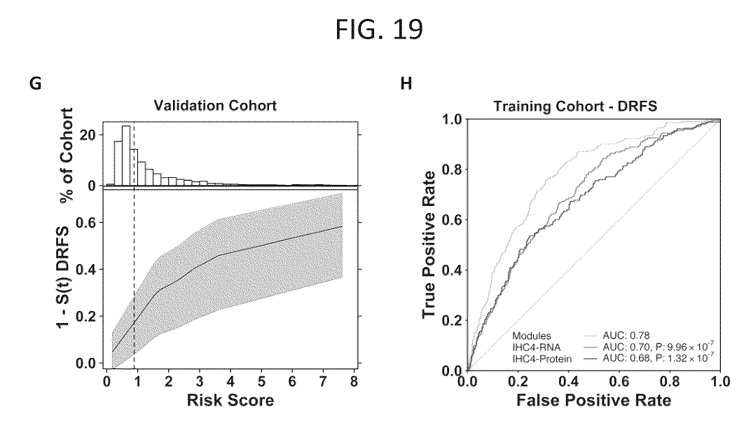

A residual risk model was generated by biomarker construction/pathway identification application 150 in the training cohort. The final signature contained four modules (i.e. modules 2, 3, 7 & 8), N-Stage and tumor size (Table 7; FIG. 19A). This signature was a robust predictor of distant metastasis in the validation cohort (FIG. 11A; Q4 vs. Q1 HR=9.68, 95% Cl: 5.91-15.84; p=2.22×10⁻⁴⁰). The signature was also effective when simply median-dichotomising predicted risk scores into low- and high-risk groups (HR=4.76; 95% Cl=3.50-6.47, p=3.19×10⁻²³, validation cohort, FIGS. 19C-D). The signature was independent of PIK3CA point-mutation data, with no change in survival curves between low and high risk groups with vs. without PIK3CA mutations (FIG. 11B; p_{Low+/-}=0.22, p_{High+/-}=0.81 FIG. 19B). Risk scores from this signature were directly correlated with the likelihood of recurrence at five years, with a higher risk score associated with a higher likelihood of metastatic event (FIGS. 11C and 19E-G).

### PIK3CA Signalling Modules Outperform Existing Markers

Finally, we compared the prognostic ability of the clinically-validated IHC4-protein model to those of our new IHC4-mRNA and PI3K signalling module models. We used the area under the receiver operating characteristic curve as a performance indicator. The PI3K pathway-based MDS model (AUC=0.75) was significantly superior to both the IHC4-mRNA (AUC=0.70; p=1.39×10⁻³) and IHC-protein (AUC=0.67; p=5.78×10⁻⁶) models (FIGS. 11D and 19H).

### DISCUSSION

By profiling key signalling nodes within the PIK3CA signalling pathway, a sixteen-gene residual risk signature adapted for theranostic use in association with early luminal breast cancer (FIG. 11A) was identified. This signature exhibits a clinically relevant and statistically significant improvement upon existing risk stratification tools, with an improved AUC from 0.67 to 0.75 (FIG. 11D) when compared with IHC4 as a benchmark.

The residual risk signature was derived using the key signalling modules in the PIK3CA signalling pathways and integration with known prognostic markers (Ki67, ER, PgR, HER2) and type I receptor tyrosine kinase signalling (EGFR, ERBB2-4). The “IHC4” markers, which assess proliferation, ER and HER2 signalling, represent a strong component of existing residual risk signatures [6].

This result establishes that molecular profiling of signalling pathways may be used for risk stratification of cancer and for patient stratification. Both the IHC4 and type I receptor tyrosine kinase modules have extensive clinical and pre-clinical data validating their utility in early breast cancer [5, 30-32]. In addition, two key nodes within the PIK3CA pathway identify TSC1/TSC2/Rheb (Module 2) and Raptor/Rictor/mTOR (Module 3) signalling nodes as of pivotal prognostic importance in early breast cancer.

Targeted therapies directed against Rheb/mTOR signalling may be of value in treatment of early luminal breast cancers. Strikingly, the collective impact of these two modules outweighed individual gene contributions from the EIF4 gene family, mediators of protein translation through CCND1/GSK3B/4EBP1 signalling, which are also associated with poor outcome in luminal cancers [33-35]. Univariate analysis of individual genes (see Table 3) indicate additional candidates for theranostic intervention in this pivotal pathway including Harvey and Kirsten RAS, PDK1 and PIK3CA itself. The documented effects of PIK3CA pathway inhibitors in advanced breast cancer, if appropriately targeted using theranostic gene/drug partnerships, may be translated into significant improvements in survival in early breast cancer. Despite the high frequency of PIK3CA mutations in this dataset [13], no prognostic impact was observed. Nor did we find any evidence that either PTEN or AKT expression, across all 3 isoforms, was important in residual risk prediction [36, 37].

### Biomarker Discovery: Additional Examples

Biomarker construction/pathway identification device 10 and patient prognosis/classification device 20 are further described with reference to further example biomarker for breast cancer, colon cancer, NSCLC cancer, and ovarian cancer. In these examples, each subnetwork module corresponds to a signaling pathway.

These example biomarkers are listed in Appendix A, and include:
(i) biomarker for breast cancer created using forward selection;
(ii) biomarker for breast cancer created using backward selection;
(iii) biomarker for colon cancer created using forward selection;
(iv) biomarker for colon cancer created using backward selection;
(v) biomarker for NSCLC cancer created using forward selection;
(vi) biomarker for NSCLC cancer created using backward selection;
(vii) biomarker for ovarian cancer created using forward selection; and
(viii) biomarker for ovarian cancer created using backward selection.

First, biomarker/pathway identification device 10 is configured and operated to construct the biomarker for the particular cancer type. Then, patient prognosis/classification device 20 is configured and operated to use the constructed biomarker to perform patient prognosis and classification for patients of the particular cancer type.

### Materials and Methods

mRNA Abundance Data Pre-Processing

As before, pre-processing was performed at biomarker construction/pathway identification device 10 by data preprocessing component 152 incorporating an R statistical environment (v2.13.0). Raw datasets from breast, colon, NSCLC and ovarian cancer studies (Tables 10-13) were normalized using RMA algorithm [70] (R package: affy v1.28.0) except for two colon cancer datasets (TOGA and Loboda dataset) which were used in their original pre-normalized and log-transformed format. ProbeSet annotation to Entrez IDs was done using custom CDFs [71] (R packages: hgu133ahsentrezgcdf v12.1.0, hgu133bhsentrezgcdf v12.1.0, hgu133plus2hsentrezgcdf v12.1.0, hthgu133ahsentrezgcdf v12.1.0, hgu95av2hsentrezgcdf v12.1.0 for breast cancer datasets. hgu133ahsentrezgcdf v14.0.0, hgu133bhsentrezgcdf v14.0.0, hgu133plus2hsentrezgcdf v14.0.0, hthgu133ahsentrezgcdf v14.0.0, hgu95av2hsentrezgcdf v14.0.0 and hu6800hsentrezgcdf v14.0.0 for the respective colon, NSCLC and ovarian cancer datasets). The Metabric breast cancer dataset was preprocessed, summarized and quantile-normalized from the raw expression files generated by Illumina BeadStudio. (R packages: beadarray v2.4.2 and illuminaHuman v3.db_1.12.2). Raw Metabric files were downloaded from European genome-phenome archive (EGA) (Study ID: EGAS00000000083). Data files of one Metabric sample were not available at the time of our analysis, and were therefore excluded. All datasets were normalized independently. Raw CEL files for mRNA abundance of TOGA ovarian cancer (Broad institute cohort) were downloaded from the TOGA data matrix (http://tcga-data.nci.nih.gov/). These were normalized using RMA (R package: affy v1.28.0) and ProbeSets were annotated to Entrez Gene IDs using custom CDF (R package: hthgu133ahsentrezgcdf v14.1.0). Pre-normalized ovarian cancer copy-number aberration and DNA methylation data was downloaded from cBio cancer genomics portal at: http://cbio.mskcc.org/cancergenomics/ov/.

For each of breast, colon, NSCLC and ovarian cancer studies, datastore 144 was populated with patient records for patients from those studies with data in the patient records normalized by data preprocessing component 152.

### Pathways Data-Preprocessing

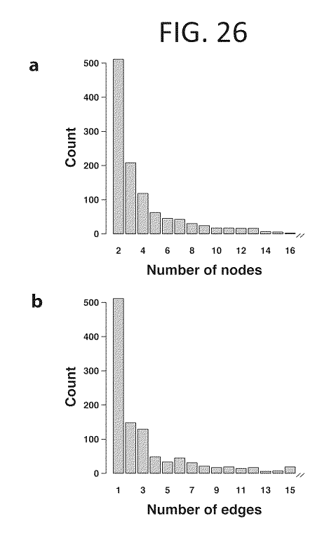

The pathway dataset was downloaded from the NCI-Nature Pathway Interaction database [72] in PID-XML format (Table 9). The XML dataset was parsed to extract protein-protein interactions from all the pathways using custom Perl (v5.8.8) scripts. The protein identifiers extracted from the XML dataset were further mapped to Entrez gene identifiers using Ensembl BioMart (version 62). Whereever annotations referred to a class of proteins, all members of the class were included in the pathway, in some case using additional annotations from Reactome and Uniprot databases. The protein-protein interactions, once mapped to the Entrez gene identifiers, were grouped under respective pathways for subsequent processing. The initial dataset contained 1,159 variable size subnetwork modules (FIGS. 26A and 26B). In order to identify redundant subnetwork modules, the overlap between all pairs of subnetwork modules was tested. When a pair of subnetwork modules had a two-way overlap above 80% (if two modules shared over 80% their network components; nodes and edges), we eliminated the smaller module. Additionally, all subnetworks modules containing less than 3 edges were excluded. In total, these criteria removed 659 subnetwork modules, resulting in 500 subnetwork modules.

TABLE 9
Overview of pathways extracted from NCI-Nature pathway interaction
database, which is an amalgamation of NCI-curated, Reactome
and BioCarta pathways databases. Protein-protein interaction
subnetworks were extracted and subsequently used to project
molecular profiles of cancer patients.

| Source | Pathways | Freeze |
|---|---|---|
| NCI-Nature curated pathways (PID) | 127 | May-11 |
| BioCarta/Reactome (PID) | 322 | May-11 |

At device 10, datastore 144 was populated with subnetwork records created for each of these 500 subnetwork modules.

### Univariate Data Analyses

In order to avoid dataset-specific bias, all included studies were analyzed independently (Table 10). First, each dataset was pre-processed independently by data preprocessing component 152, as described in the ThRNA abundance data pre-processing′ section above. Next, genes across all the datasets were evaluated for their prognostic power using a univariate Cox proportional hazards model followed by the Wald-test (R package: survival v2.36-9). Overall survival (OS) was used as the survival time variable; for the studies that do not report OS, the closest alternative endpoint available in that study was used (e.g. disease-specific survival or distant metastasis-free survival). All the genes were subsequently ranked by the Wald-test p-value within each study. The top genes across all studies were compared on multiple criterion:

### 1—Rank Product

The Rank Product [73] of each gene was computed as:

$$R {P}_{g} = {\sum }_{i = 1}^{k} {log ( {r}_{gi} )}^{\frac{1}{k}} \qquad ( 1 )$$

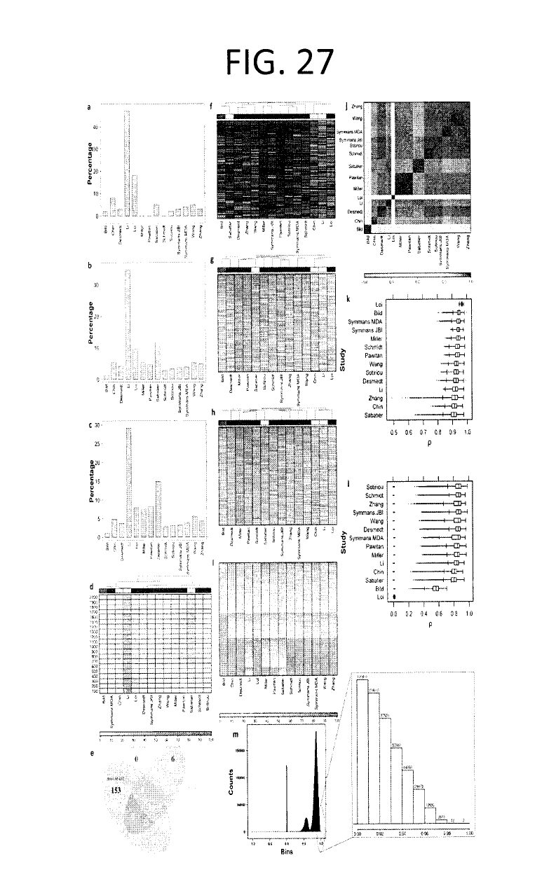

Here k represents the number of studies which had the mRNA abundance measure available for gene g. rᵢ is the rank of gene g in study i. The overall ranking table was used as a benchmark to identify datasets in which a given gene was ranked farthest when its rank product was compared to studywise ranks. The farthest dataset count was computed for the overall top ranked (100, 200, 300, . . . , 1000, 2000) genes (FIGS. 27A-E).

### 2—Percentile Ranks

The p-value (Wald-test) based ranking was transformed into percentile ranks within each study. These ranks were used as a measure of gene's position with reference to the benchmark rank derived in the step 1 to evaluate deviation of genes' ranks for each study (FIGS. 27F-L).

TABLE 10
List of breast cancer studies included in preliminary analysis
[114-126]. Li et al. and Loi et al. were regarded as outliers
following univariate analyses (FIG. 27), and subsequently removed
from further analyses. The remaining studies were divided into
two groups to keep a modest balance in the size and array platform
distribution for training and testing of prognostic models.

| | Patients | | | | |
|---|---|---|---|---|---|
| | with | | | | |
| | Survival | | Array | Analysis | |
| Study | Data | Genes | Platform | Group | Year |
| Bild et al. | 158 | 8260 | HG-U95AV2 | Validation | 2006 |
| Chin et al. | 129 | 11972 | HTHG-U133A | Validation | 2006 |
| Desmedt et al. | 198 | 11979 | HG-U133A | Training | 2007 |
| Li et al. | 115 | 17788 | HG-U133- | Excluded | 2010 |
| | | | PLUS2 | | |
| Loi et al. | 77 | 11979 | HG-U133A | Excluded | 2008 |
| Miller et al. | 236 | 16600 | HG-U133A/B | Validation | 2005 |
| Pawitan et al. | 159 | 16600 | HG-U133A/B | Training | 2005 |
| Sabatier et al. | 252 | 17788 | HG-U133- | Training | 2010 |
| | | | PLUS2 | | |
| Schmidt et al. | 200 | 11979 | HG-U133A | Training | 2008 |
| Sotiriou et al. | 94 | 11979 | HG-U133A | Validation | 2006 |
| Symmans et al. | 65 | 11979 | HG-U133A | Training | 2010 |
| (JBI) | | | | | |
| Symmans et al. | 195 | 11979 | HG-U133A | Validation | 2010 |
| (MDA) | | | | | |
| Wang et al. | 286 | 11979 | HG-U133A | Validation | 2005 |
| Zhang et al. | 136 | 11979 | HG-U133A | Training | 2009 |

### 3—Intra- and Inter-Study Correlation

The mRNA abundance profiles of common genes across all studies were extracted and patient wise Spearman rank correlation coefficient was estimated (R package: stats v2.13.0). The correlation coefficient was used to further analyze intra- and inter-study correlation in order to identify any outlier studies (FIGS. 27J-L).

Eliminating Redundant mRNA Profiles (Breast Cancer Data)

The Spearman rank correlation coefficient was also used to establish a non-redundant set of patients. This is important not only to identify any patients that might have participated in more than one study or duplicate data used in multiple papers, but also to train a robust model thereby preventing model over-fitting. The survival data of patients with high correlation coefficient (ρ≧0.98) was matched, and 22 samples [65, 74] having identical survival time and status were found. These patients were removed from further analyses (FIG. 27M).

Correspondingly, patient records in datastore 144 were updated to remove records for redundant patients.

### Meta-Analysis

Following univariate analyses and elimination of redundant patients, the remaining studies were divided into two sets, training and validation (Tables 10-13). The RMA normalized mRNA abundance measures were median scaled within the scope of each dataset (R package: stats v2.13.0) by data preprocessing component 152.

### 1—Gene Hazard Ratio

At device 10, models were fitted to the patient records by model construction component 160. The hazard ratio for all the genes by combining samples from all the training datasets was estimated using the univariate Cox proportional hazards model. The Cox model was fit to the median dichotomized grouping of mRNA abundance profiles of the samples as opposed to continuous measure of mRNA abundance.

### 2—Interaction Hazard Ratio

The hazard ratio for all the protein-protein interactions gathered from the NCI-Nature pathway interaction database were estimated using a multivariate Cox proportional hazards model. A Cox model, shown below, was fit to median dichotomized patient grouping of each of the interacting gene pairs:

h(t)=h₀(t)exp(β₁X_{G1}+β₂X_{G2}/β₃X_{G1.G2}) (2)

where X_{G1} and X_{G2} represent patient's group for gene 1 and gene 2. X_{G1.G2} represents patient's binary interaction measure between the gene 1 and gene 2, as shown below:

X_{G1.G2}=(G̅1̅⊕̅G̅2̅) (3)

where ⊕ represents exclusive disjunction between the grouping of each gene. The expression encodes XNOR boolean function emulating true (1) whenever both the interacting genes belong to the same group.

### Subnetwork Module-Dysregulation Score (MDS)

At device 10, module scoring component 154 processed patient records and subnetwork records stored in datastore 144 to score each of the modules. In particular, the pathway-based subnetwork modules were scored using three different models. These models compute a module-dysregulation score (MDS) by incorporating the hazard ratio of nodes and edges that form the subnetwork:

$$\begin{matrix}1 \text{-} Nodes + Edges & \\ MDS = {\sum }_{i = 1}^{n} \vert {log}_{2} {HR}_{i} \vert + {\sum }_{j = 1}^{e} \vert {log}_{2} {HR}_{j} \vert & ( 4 ) \\ 2 \text{-} Nodes only & \\ MDS = {\sum }_{i = 1}^{n} \vert {log}_{2} {HR}_{i} \vert & ( 5 ) \\ 3 \text{-} Edges only & \\ MDS = {\sum }_{j = 1}^{e} \vert {log}_{2} {HR}_{j} \vert & ( 6 )\end{matrix}$$

where n and e represent total number of nodes (genes) and edges (interactions) in a subnetwork module respectively. HR represents the hazard ratios of genes and the protein-protein interactions in a subnetwork module (section: Meta-analysis). The subnetworks were ranked by module ranking component 156 according to their MDS, thereby identifying candidate prognostic features.

### Patient Risk Score

The subnetwork MDS was used to draw a list of the top n subnetwork features for each of the three models (see section: Subnetwork module-dysregulation score). These features were subsequently used to estimate patient risk scores using Model N+E, N and E. The patient risk score for each of the subnetwork modules (risk_{SN}) was expressed using the following models constructed by model construction component 160:

$$\begin{matrix}1 \text{-} Nodes + Edges & \\ {risk}_{SN} = {\sum }_{i = 1}^{n} ( {log}_{2} {HR}_{i} ) {ω}_{i} + {\sum }_{j = 1}^{e} ( {log}_{2} {HR}_{j} ) {ω}_{{j}_{x}} {ω}_{{j}_{y}} & ( 7 ) \\ 2 \text{-} Nodes only & \\ {risk}_{SN} = {\sum }_{i = 1}^{n} ( {log}_{2} {HR}_{i} ) {ω}_{i} & ( 8 ) \\ 3 \text{-} Edges only & \\ {risk}_{SN} = {\sum }_{j = 1}^{e} ( {log}_{2} {HR}_{j} ) {ω}_{{j}_{x}} {ω}_{{j}_{y}} & ( 9 )\end{matrix}$$

where n and e represent the total number of nodes (genes) and edges (interactions) in a subnetwork module (SN), respectively. HR is the hazard ratio of genes and the protein-protein interactions (section: Meta-analysis) in a subnetwork module. x and y are the two nodes connected by an edge eⱼ and ω is the scaled intensity of an arbitrary molecular profile (e.g. mRNA abundance, copy number aberrations, DNA methylation beta values etc).

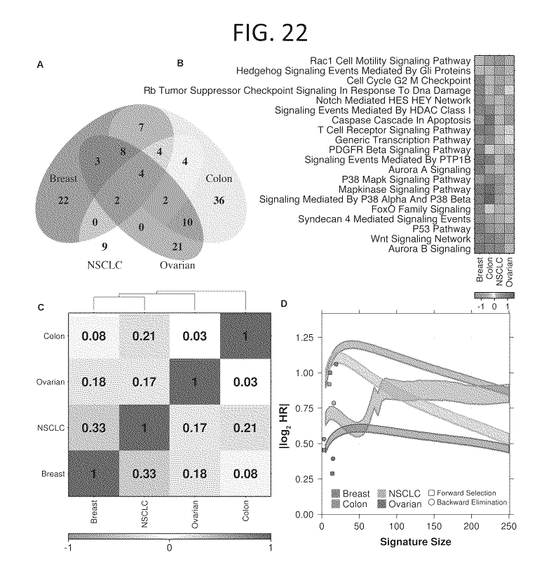

A univariate Cox proportional hazards model was fitted to the training set by model construction component 160, and applied to the validation set for each of the subnetwork modules. The prognostic power of all three models was compared using non-parametric two sample Wilcoxon rank-sum test (R package: stats v2.13.0) (FIGS. 22C and 22D).

### Subnetwork Feature Selection

In order to narrow down the size of subnetwork features in each of the three models yet maintaining the prognostic power, backward variable elimination and forward variable selection algorithms was applied by module selection component 158. The backward elimination algorithm starts with a model having a complete feature set and attempts to remove the least informative features one by one, as long as the overall performance is not compromised. Conversely, the forward selection algorithm starts with the most prognostic feature and expands the model by adding one feature at a time. Both models terminate as soon as the overall performance is locally maximized. Following every addition or deletion, the model re-computes the goodness of fit, called Akaike information criterion (AIC). The AIC measure guides the model on the statistical significance of a feature/variable in consideration. The selection/elimination trace was tracked from the beginning to the convergence point and, at each iteration, the prognostic power for that particular state of the model was evaluated (R package: MASS v7.3-12). The evaluation was conducted by fitting a multivariate Cox proportional hazards model on the training set. The coefficients (β) estimated by the fit were subsequently used to compute an overall measure of per patient risk score for the validation set using the following formula:

$${risk}_{i} = {\sum }_{j = 1}^{m} {β}_{j} ( {Y}_{ij} ) \qquad ( 10 )$$

where Yᵢⱼ is the i^{th} patient's risk score for subnetwork module j. The training set HRs of the nodes and edges were used to compute Yᵢⱼ (see section: Patient risk score). Next, the validation cohort was median dichotomized into low- and high-risk patients using the median risk score estimated on the training set. The risk group classification was assessed for potential association with patient survival data using Cox proportional hazards model and Kaplan-Meier survival analysis.

The biomarker is the selected subset of the subnetwork modules following backward variable elimination/forward variable selection.

### Model Comparison

The performance comparison of all three models was conducted by bootstrapping training set samples 10,000 times. Each model was tested on the validation set samples. Validation results of Model N+E, N, and E were compared using Tukey HSD test (R package: stats v2.13.0).

### Randomization of Candidate Subnetwork Markers

Jackknifing was performed over the subnetwork marker space for four tumour types; breast, colon, NSCLC and ovarian. Ten million prognostic classifiers (200,000 for each size n=5, 10, 15, . . . , 250; where n represents the number of subnetworks) were randomly sampled using all 500 subnetworks. The predictive performance of each random classifier was measured as the absolute value of the log₂-transformed hazard ratio obtained by fitting a multivariate Cox proportional hazards model using Model N.

### Visualizations

All plots were created in the R statistical environment (v2.13.0). Forest plots were generated using rmeta package (v2.16), all others were created using lattice (v0.19-28), latticeExtra (v0.6-16) and VennDiagram (v1.0.0) packages.

### Univariate Analyses Reveal Outliers and Duplicate Profiles

At device 10, 14 mRNA abundance breast cancer datasets were collated (Table 10). Since these datasets originate from different studies and array platforms, comprehensive univariate analyses were conducted to identify outlier datasets and to find patients duplicated across datasets. Two studies were identified as outliers and 22 redundant patients having identical survival data (FIG. 27). Outlier detection was grounded on inter-study expression correlation and prognostic ranking of genes, while the redundant samples were common donors between studies. These were removed from further processing, leaving 12 cohorts with 2,108 patients. These were divided into training (6 studies, 1,010 patients) and testing sets (6 studies, 1,098 patients). The testing set is fully independent and does not overlap with the training set. Cohorts of primary colon, lung and ovarian cancer patient mRNA profiles were assembled in similar ways, however, without outlier detection due to relatively small number of publicly available datasets (Tables 11-13).

Comparison with Colon, NSCLC and Ovarian Cancer Prognostic Biomarkers

In order to compare the performance of SIMMS's with existing gene expression-based colon [99, 100], NSCLC [101-105] and ovarian [106-109] cancer prognostic biomarkers, we limited our search to the studies which shared the validation datasets with those included in our analysis as validation datasets too. This selection criterion enabled unbiased comparison of hazard ratios and P-values between published markers and those identified by SIMMS for the same set of patients unless specified otherwise. To maintain parity, strictly gene expression-based predictors with dichotomous output were considered for performance evaluation. These results are presented in Table 26. To test the colon cancer 34-gene signature [100] on TCGA cohort, this signature was re-implemented following the original protocol. Briefly, VMC and Moffitt sub-cohorts were treated as training and validation sets respectively. The validation results on the Moffitt cohort and TCGA cohort are shown in Table 26.

Comparison with Oncotype DX and MammaPrint

Oncotype DX is an RT-PCR 21-gene signature having 5 normalization genes and 16 predictor genes [110]. Of the 16 predictor genes, Entrez gene 2944 was missing from all validation datasets and Entrez gene 57758 was missing from the Bild dataset. Entrez gene 6175 was missing from the normalization genes. These missing genes were assigned zero score. The mRNA profiles of the predictor genes were normalized by subtracting the mean of normalization gene set. The original Oncotype DX protocol was implemented using R package genefu (v1.2.1) [111]. The Oncotype DX protocol offers 3 risk groups; low (risk score<18), intermediate (18 risk score<31) and high 31). To make it comparable with SIMMS, the intermediate risk group patients was split into low- and high-risk groups at the median of risk score guide for the intermediate group (24.5). The dichotomized groups across all validation datasets were further analyzed using Cox proportional hazards model followed by Kaplan-Meier analysis (Table 8).

TABLE 8
Comparison of SIMMS (Model N) with clinically validated biomarkers for 10-year survival.
The Cox proportional hazard model's p (Wald-test) was used as an indicator of performance
comparison across all validation studies independently as well as combined validation cohort.
The p-values and HR for SIMMS (top n_{Breast} = 50) are reported for comparison.
Oncotype DX and MammaPrint classifiers were applied to the patients in SIMMS validation
cohorts, and corresponding p-values and HR are presented here.

| | SIMMS | | |
|---|---|---|---|
| | (Model N, n = 50) | OncotypeDX | |
| Study | Backward | Cutoff score = | |
| (Patients) | elimination | 24.5 | MammaPrint |
| Bild et al. (158) | 0.08 (1.69) | 1 (NA) | 0.33 (2.65) |
| Chin et al. (129) | 0.008 (2.36) | 0.32 (2.06) | 0.23 (1.70) |
| Miller et al. (236) | 9.52 × 10⁻⁴ (2.65) | 0.14 (2.15) | 0.001 (5.30) |
| Sotiriou et al. (94) | 0.02 (3.08) | 0.16 (4.20) | 1 (NA) |
| Symmans et al. | 1.35 × 10⁻⁴ (3.75) | 0.31 (2.08) | 0.2 (2.14) |
| (MDA) (195) | | | |
| Wang et al. (286) | 0.02 (1.58) | 0.01 (4.34) | 0.002 (2.61) |
| Curtis et al. - Metabric | 2.05 × 10⁻⁶ (1.43) | 4.32 × 10⁻¹⁰ (1.75) | 5.82 × 10⁻⁶ (1.66) |
| cohort (1988) | | | |

MammaPrint is a microarray based 70-gene signature [112]. Of the 70 genes, we were unable to map 7 genes to Entrez ids in our validation cohort, namely Contig32125_RC, Contig20217_RC, Contig24252_RC, Contig40831_RC, Contig35251_RC, AA555029_RC and Contig63649_RC. We set the corresponding mRNA abundance score of these genes to zero. The gene signature implementation was done using R package genefu (v1.2.1) [111]. The risk scores were dichotomized by using two different thresholds; default (0.3) and median risk score (Table 8).

For both Oncotype DX and MammaPrint, due to limited clinical annotations for

Affymetrix based datasets, we used all patients. However, for Metabric (Illumina dataset), Oncotype DX was applied to preselected Stage [0,1,2,3], ER positive, lymph node negative and HER2 negative patients only. Similarly MammaPrint was applied to Stage [0,1,2], lymph node negative patients having tumour size<5 cm.

Overall, SIMMS performance was at least as good as MammaPrint and better than Oncotype DX across the studies in validation cohort, independently as well as combined.

### Integrating Multiple Datatypes of TOGA Ovarian Cancer

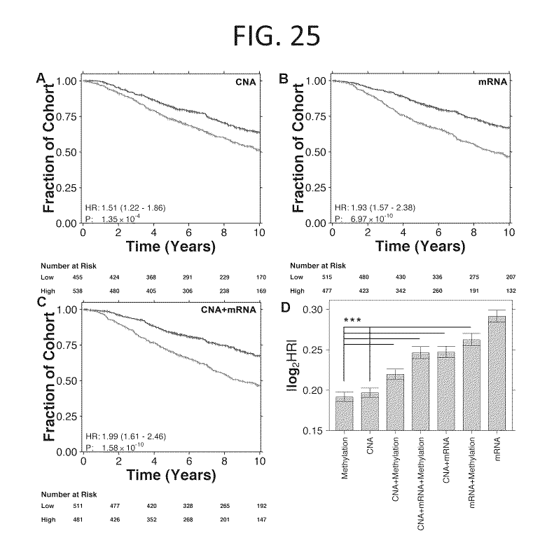

Recent studies conducted by TOGA have generated datasets on multiple genomic aberrations including somatic mutations, mRNA abundance, copy-number aberration (CNA) and DNA methylation [107, 113]. These datasets lend themselves naturally to integrative analyses that are crucial to bridge the gap between molecular features and clinical covariates. To this end, we applied our methodology to TOGA ovarian cancer [107] (Broad Institute cohort) and established 7 different models using SIMMS Model N. Molecular features based on mRNA, CNA and DNA methylation were used as gene-level properties. Next, subnetwork modules feature selection was carried out and MDS was computed by using the above-mentioned features independently as well as in a multivariate setting. As we only had one dataset with 478 patients having all three data types, the dataset was randomly dichotomized into equal sized training and validation cohorts. To avoid randomization specific bias, the procedure was repeated 1,000 times and aggregated the validation results (FIG. 25D). We observed that in addition to mRNA-derived model, multimodal mRNA+DNA methylation, CNA+mRNA and CNA+mRNA+DNA methylation models were better predictors of patient outcome compared to unimodal CNA and DNA methylation models (all pairwise comparisons: p<0.001 Welch's unpaired t-test) (FIG. 25D). These results underline the benefits of integrating multiple data types.

### SIMMS R Package

SIMMS, as for example implemented in biomarker construction/pathway identification application 150, is generic and can work with any combination of molecular features and interaction networks. In an embodiment, it provides an extendible framework to support user-defined parameter estimation and classification algorithms. In an embodiment, SIMMS provides: (i) support for multiple datatypes (mRNA, methylation, CNA etc), (ii) support for user-defined networks, and (iii) support for user-defined methods for quantifying dysregulation effect of a subnetwork. For (i), users can supply the location and names of the files they would like to analyze with SIMMS. For (ii), a text file describing networks in a tab-delimited format can be supplied as an input to SIMMS, see pathway_based—networks*.txt files that comes as a part of R package. For (iii), the package offers an interface function ‘derive.network.features’ that accepts a parameter ‘feature.selection.fun’ for user-defined function name (see code snippet below). By default, the function ‘calculate.network.coefficients’ is called to compute MDS for Mode N, Model E and Mode N+E. However, users can easily write their own algorithms and simply use them with SIMMS as plug and play components.

derive.network.features <− function(
data.directory = “.”, output.directory = “.”, data.types = c(“mRNA”),
feature.selection.fun = “calculate.network.coefficients”,
feature.selection.datasets = NULL, feature.selection.p.thresholds = c(0.05), subset = NULL, ...
);

### DISCUSSION

### Overview of SIMMS Prioritization of Candidate Prognostic Markers

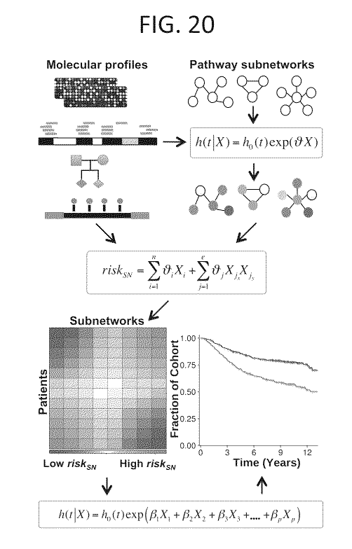

SIMMS, as implemented for example in biomarker construction/pathway identification application 150, acts upon a collection of subnetwork modules, where each node is a molecule (e.g. a gene or metabolite) and each edge is an interaction (physical or functional) between molecules. Molecular data is projected onto these subnetworks using network topology measurements that represent the impact of and synergy between different molecular features and associated patient data. Because different biological processes can have different underlying tumourigenic promoting network architectures, three network topology measurements are provided based on different interaction models. One model, hereafter referred to as Model N (nodes only), estimates the extent of dysregulation in molecules that function together. Two other models Model E (edges only) and Model N+E (nodes and edges) incorporate the impact of dysregulated interactions (Methods). Regardless of which model is used, module scoring component 154 of application 150 computes a Thodule-dysregulation score′ (MDS) for each subnetwork that measures how a disease affects any given subnetwork (FIG. 20). SIMMS as implemented in application 150 was evaluated using a collection of 449 gene-centric pathways from the high-quality, manually-curated NCI-Nature Pathway Interaction database [72]. These pathways comprise 500 non-overlapping subnetworks, hereafter referred to as subnetwork modules (Table 9, FIG. 26). We then fit the SIMMS model to integrated datasets of primary breast, colon, NSCLC and ovarian cancers (Tables 10-13, FIG. 27).

### Topological Characteristics of Candidate Prognostic Subnetworks

We first focused on prognostic models, which predict patient survival, and therefore used Cox proportional hazards models for these censored data. Each Cox model generated a hazard ratios (HR) which quantifies how effectively a biomarker can stratify patients into low- and high-risk groups (Methods).

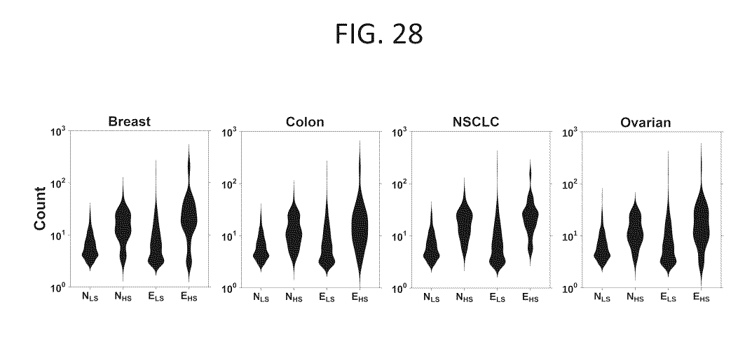

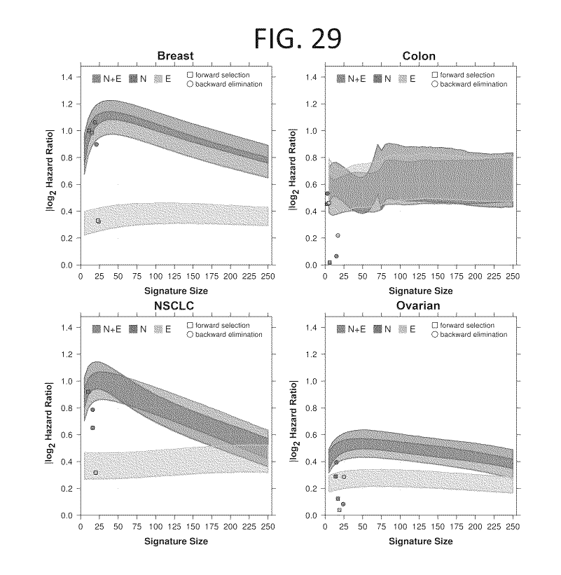

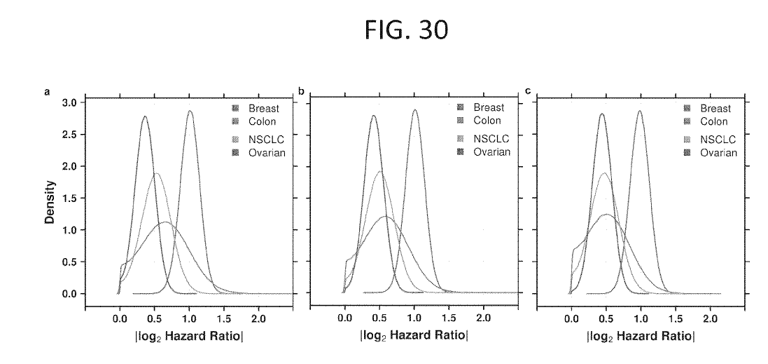

The distributional characteristics of our candidate disease-subnetwork modules revealed unexpected and important properties of tumour network biology. First, there was a global propensity for highly prognostic subnetworks to be larger, containing more genes and interactions than expected by chance (nodes p<10⁻³, edges p<10⁻³; permutation test) (FIG. 28). This strong correlation between subnetwork size and MDS was consistent across all cancer types studied, even though different pathways were altered in each. This indicates common mechanistic processes underlying tumour evolution. This is concordant with data showing that oncogenic subnetworks are extensively deregulated, with mutations affecting the sequences and expression of hundreds of genes [75]. Second, we used a large-scale permutation study in the training cohort to characterize the null distribution of the subnetwork-modules scored by SIMMS in each disease (FIG. 29). We found that large numbers of randomly-generated subnetworks had prognostic potential, particularly in breast and lung cancer, as reported previously [76-78]. Interestingly, different tumour types showed very different null distributions, indicating that the number and nature of pathways altered in each tumour type is distinct (FIG. 30).

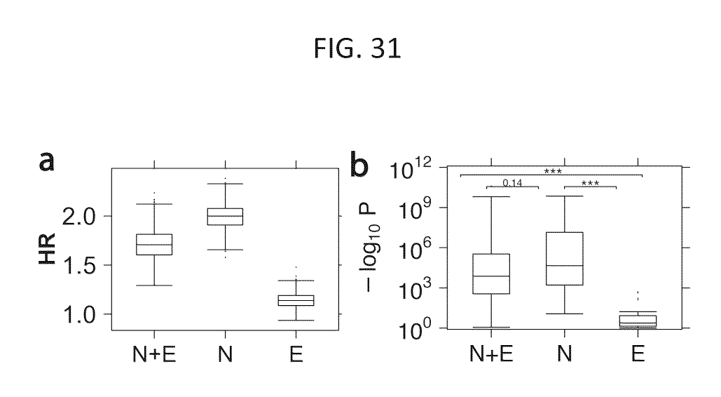

To ensure independence from the discovery cohort-specific effects, we inspected prediction robustness by permuting the discovery cohorts. While a distribution of performance was observed both in terms of statistical significance (FIG. 31A) and effect-size (FIG. 31B), statistically significant prognostic subnetworks were identified in all cases. Of the three models, Model N was consistently more prognostic than models N+E or E, we therefore focused solely on Model N moving forward (one-way ANOVA with Tukey's HSD multiple comparison test, p<0.001) (Tables 14-17, 22-25).

TABLE 14
Breast cancer Model N + E. Hazard ratios (95% CI, p values, size of the validation
cohort and q values) of patients' MDS based classification. A univariate Cox proportional
hazards model was fit to each of the top ranked subnetwork markers (n_{Breast} = 50, n_{Colon} =
75, n_{NSCLC} = 25 and n_{Ovarian} = 50) and subsequently applied to predict patient
risk score in the validation cohort. The survival differences between the predicted
groups were assessed using Kaplan-Meier analysis.

| | | 95% CI | 95% CI | | | |
|---|---|---|---|---|---|---|
| Subnetwork module | HR | lower | upper | P | n | Q |
| X.ID.200144_1.NAME.PDGFR.beta.signaling. | 2.181 | 1.735 | 2.742 | 2.452E−11 | 1098 | 1.226E−09 |
| pathway | | | | | | |
| X.ID.200006_1.NAME.Signaling.events. | 2.088 | 1.667 | 2.616 | 1.546E−10 | 1098 | 3.0653E−09 |
| mediated.by.PRL | | | | | | |
| X.ID.200097_1.NAME.PLK1.signaling.events | 2.082 | 1.662 | 2.609 | 1.839E−10 | 1098 | 3.0653E−09 |
| X.ID.200040_1.NAME.Signaling.events. | 2.122 | 1.681 | 2.679 | 2.468E−10 | 1098 | 3.0854E−09 |
| mediated.by.PTP1B | | | | | | |
| X.ID.100022_1.NAME.t.cell.receptor.signaling. | 2.035 | 1.617 | 2.561 | 1.362E−09 | 1098 | 1.3618E−08 |
| pathway | | | | | | |
| X.ID.501001_1.NAME.Mitotic.Telophase.. | 1.991 | 1.589 | 2.494 | 2.148E−09 | 1098 | 1.7903E−08 |
| Cytokinesis | | | | | | |
| X.ID.200187_1.NAME.Aurora.A.signaling | 1.942 | 1.554 | 2.427 | 5.432E−09 | 1098 | 3.8799E−08 |
| X.ID.200011_1.NAME.Aurora.B.signaling | 1.831 | 1.464 | 2.289 | 1.148E−07 | 1098 | 7.1765E−07 |
| X.ID.100226_1.NAME.bioactive.peptide. | 1.833 | 1.462 | 2.298 | 1.511E−07 | 1098 | 8.394E−07 |
| induced.signaling.pathway | | | | | | |
| X.ID.200173_1.NAME.Signaling.mediated. | 1.808 | 1.442 | 2.266 | 2.848E−07 | 1098 | 1.4241E−06 |
| by.p38.alpha.and.p38.beta | | | | | | |
| X.ID.200081_2.NAME.Regulation.of.Telomerase | 1.738 | 1.386 | 2.181 | 1.77E−06 | 1098 | 8.0433E−06 |
| X.ID.500866_1.NAME.mRNA.Splicing... | 1.735 | 1.378 | 2.183 | 2.655E−06 | 1098 | 1.1063E−05 |
| Major.Pathway | | | | | | |
| X.ID.200190_1.NAME.Class.I.PI3K.signaling. | 1.717 | 1.369 | 2.154 | 2.971E−06 | 1098 | 1.1428E−05 |
| events.mediated.by.Akt | | | | | | |
| X.ID.200003_1.NAME.Fc.epsilon.receptor. | 1.697 | 1.355 | 2.126 | 4.189E−06 | 1098 | 1.496E−05 |
| I.signaling.in.mast.cells | | | | | | |
| X.ID.100113_1.NAME.mapkinase.signaling. | 1.684 | 1.345 | 2.108 | 5.383E−06 | 1098 | 1.7942E−05 |
| pathway | | | | | | |
| X.ID.200199_1.NAME.p53.pathway | 1.645 | 1.312 | 2.061 | 1.561E−05 | 1098 | 4.8795E−05 |
| X.ID.500379_1.NAME.Polo.like.kinase. | 1.627 | 1.301 | 2.035 | 1.956E−05 | 1098 | 5.6265E−05 |
| mediated.events | | | | | | |
| X.ID.200102_1.NAME.FoxO.family.signaling | 1.638 | 1.305 | 2.055 | 2.026E−05 | 1098 | 5.6265E−05 |
| X.ID.200064_1.NAME.Wnt.signaling.network | 1.612 | 1.289 | 2.016 | 2.91E−05 | 1098 | 7.659E−05 |
| X.ID.100029_1.NAME.sprouty.regulation. | 1.6 | 1.281 | 1.997 | 3.407E−05 | 1098 | 8.5173E−05 |
| of.tyrosine.kinase.signals | | | | | | |
| X.ID.200048_1.NAME.Calcineurin.regulated. | 1.595 | 1.273 | 1.999 | 4.949E−05 | 1098 | 0.00011783 |
| NFAT.dependent.transcription.in.lymphocytes | | | | | | |
| X.ID.200208_2.NAME.Downstream.signaling. | 1.58 | 1.263 | 1.976 | 6.119E−05 | 1098 | 0.00013907 |
| in.naive.CD8..T.cells | | | | | | |
| X.ID.200098_1.NAME.Ras.signaling.in.the. | 1.575 | 1.258 | 1.97 | 7.298E−05 | 1098 | 0.00015866 |
| CD4..TCR.pathway | | | | | | |
| X.ID.200070_3.NAME.LKB1.signaling.events | 1.553 | 1.242 | 1.941 | 0.0001106 | 1098 | 0.00023041 |
| X.ID.200079_1.NAME.Signaling.events. | 1.555 | 1.24 | 1.95 | 0.000133 | 1098 | 0.00025609 |
| mediated.by.HDAC.Class.I | | | | | | |
| X.ID.100119_1.NAME.keratinocyte.differentiation | 1.561 | 1.242 | 1.963 | 0.000136 | 1098 | 0.00025609 |
| X.ID.100245_2.NAME.akt.signaling.pathway | 1.543 | 1.235 | 1.929 | 0.0001383 | 1098 | 0.00025609 |
| X.ID.200081_1.NAME.Regulation.of.Telomerase | 1.541 | 1.233 | 1.927 | 0.0001472 | 1098 | 0.00026289 |
| X.ID.100101_1.NAME.mtor.signaling.pathway | 1.531 | 1.227 | 1.911 | 0.0001657 | 1098 | 0.00028571 |
| X.ID.200077_1.NAME.Circadian.rhythm. | 1.521 | 1.22 | 1.898 | 0.0001995 | 1098 | 0.00033252 |
| pathway | | | | | | |
| X.ID.200158_1.NAME.Retinoic.acid.receptors. | 1.498 | 1.201 | 1.87 | 0.0003462 | 1098 | 0.00055834 |
| mediated.signaling | | | | | | |
| X.ID.200206_1.NAME.Trk.receptor.signaling. | 1.491 | 1.194 | 1.861 | 0.0004161 | 1098 | 0.00064864 |
| mediated.by.the.MAPK.pathway | | | | | | |
| X.ID.100152_1.NAME.inactivation.of.gsk3. | 1.49 | 1.193 | 1.859 | 0.0004281 | 1098 | 0.00064864 |
| by.akt.causes.accumulation.of.b.catenin. | | | | | | |
| in.alveolar.macrophages | | | | | | |
| X.ID.100084_1.NAME.hypoxia.and.p53. | 1.49 | 1.19 | 1.865 | 0.000505 | 1098 | 0.00074268 |
| in.the.cardiovascular.system | | | | | | |
| X.ID.200215_2.NAME.Regulation.of.retinoblastoma. | 1.479 | 1.185 | 1.846 | 0.000529 | 1098 | 0.00075578 |
| protein | | | | | | |
| X.ID.200220_1.NAME.Notch.mediated. | 1.481 | 1.183 | 1.854 | 0.0006117 | 1098 | 0.00084962 |
| HES.HEY.network | | | | | | |
| X.ID.200166_2.NAME.Caspase.cascade. | 1.477 | 1.181 | 1.847 | 0.0006353 | 1098 | 0.0008585 |
| in.apoptosis | | | | | | |
| X.ID.200076_2.NAME.FAS..CD95..signaling. | 1.408 | 1.125 | 1.761 | 0.0027674 | 1098 | 0.00364127 |
| pathway | | | | | | |
| X.ID.200126_2.NAME.ErbB1.downstream. | 1.395 | 1.118 | 1.741 | 0.0031685 | 1098 | 0.00406223 |
| signaling | | | | | | |
| X.ID.200112_1.NAME.IL2.signaling.events. | 1.391 | 1.115 | 1.735 | 0.0034699 | 1098 | 0.0043374 |
| mediated.by.PI3K | | | | | | |
| X.ID.200128_1.NAME.Syndecan.4.mediated. | 1.377 | 1.103 | 1.718 | 0.0046459 | 1098 | 0.00566568 |
| signaling.events | | | | | | |
| X.ID.100218_1.NAME.caspase.cascade. | 1.364 | 1.091 | 1.705 | 0.0064775 | 1098 | 0.0077113 |
| in.apoptosis | | | | | | |
| X.ID.100144_1.NAME.hiv.1.nef..negative. | 1.316 | 1.055 | 1.642 | 0.0148273 | 1098 | 0.01695248 |
| effector.of.fas.and.tnf | | | | | | |
| X.ID.100085_1.NAME.p38.mapk.signaling. | 1.315 | 1.055 | 1.639 | 0.0149182 | 1098 | 0.01695248 |
| pathway | | | | | | |
| X.ID.200132_1.NAME.AP.1.transcription. | 1.282 | 1.029 | 1.597 | 0.0265059 | 1098 | 0.02945099 |
| factor.network | | | | | | |
| X.ID.100123_1.NAME.integrin.signaling. | 1.27 | 1.02 | 1.582 | 0.0325928 | 1098 | 0.03542698 |
| pathway | | | | | | |
| X.ID.500655_1.NAME.Processing.of.Capped. | 1.263 | 1.011 | 1.578 | 0.0395854 | 1098 | 0.04211209 |
| Intron.Containing.Pre.mRNA | | | | | | |
| X.ID.100132_1.NAME.signal.transduction. | 1.234 | 0.991 | 1.537 | 0.0602669 | 1098 | 0.06277802 |
| through.il1r | | | | | | |
| X.ID.500652_1.NAME.Generic.Transcription. | 1.075 | 0.862 | 1.342 | 0.519708 | 1098 | 0.53031424 |
| Pathway | | | | | | |
| X.ID.100026_2.NAME.tnf.stress.related. | 1.018 | 0.817 | 1.268 | 0.873819 | 1098 | 0.87381898 |
| signaling | | | | | | |

TABLE 14
Breast cancer Model N. Hazard ratios (95% CI, p values, size of the validation cohort
and q values) of patients' MDS based classification. A univariate Cox proportional hazards
model was fit to each of the top ranked subnetwork markers (n_{Breast} = 50, n_{Colon} = 75,
n_{NSCLC} = 25 and n_{Ovarian} = 50) and subsequently applied to predict patient
risk score in the validation cohort. The survival differences between the predicted
groups were assessed using Kaplan-Meier analysis.

| | | 95% CI | 95% CI | | | |
|---|---|---|---|---|---|---|
| Subnetwork module | HR | lower | upper | P | n | Q |
| X.ID.200040_1.NAME.Signaling. | 2.133 | 1.693 | 2.689 | 1.38E−10 | 1098 | 6.92E−09 |
| events.mediated.by.PTP1B | | | | | | |
| X.ID.200097_1.NAME.PLK1. | 2.074 | 1.653 | 2.603 | 2.95E−10 | 1098 | 7.37E−09 |
| signaling.events | | | | | | |
| X.ID.500991_1.NAME.Cyclin. | 2.025 | 1.62 | 2.532 | 5.88E−10 | 1098 | 7.96E−09 |
| A.B1.associated.events.during. | | | | | | |
| G2.M.transition | | | | | | |
| X.ID.500328_1.NAME.Inactivation. | 2.038 | 1.626 | 2.555 | 6.36E−10 | 1098 | 7.96E−09 |
| of.APC.C.via.direct.inhibition. | | | | | | |
| of.the.APC.C.complex | | | | | | |
| X.ID.200187_1.NAME.Aurora. | 2.001 | 1.598 | 2.506 | 1.45E−09 | 1098 | 1.45E−08 |
| A.signaling | | | | | | |
| X.ID.200011_1.NAME.Aurora. | 1.973 | 1.577 | 2.469 | 2.80E−09 | 1098 | 2.01E−08 |
| B.signaling | | | | | | |
| X.ID.200006_1.NAME.Signaling. | 1.971 | 1.576 | 2.466 | 2.82E−09 | 1098 | 2.01E−08 |
| events.mediated.by.PRL | | | | | | |
| X.ID.100113_1.NAME.mapkinase. | 1.988 | 1.58 | 2.5 | 4.40E−09 | 1098 | 2.75E−08 |
| signaling.pathway | | | | | | |
| X.ID.501001_1.NAME.Mitotic. | 1.922 | 1.535 | 2.406 | 1.21E−08 | 1098 | 6.42E−08 |
| Telophase..Cytokinesis | | | | | | |
| X.ID.100022_1.NAME.t.cell.receptor. | 1.934 | 1.541 | 2.429 | 1.33E−08 | 1098 | 6.42E−08 |
| signaling.pathway | | | | | | |
| X.ID.100226_1.NAME.bioactive. | 1.928 | 1.537 | 2.42 | 1.41E−08 | 1098 | 6.42E−08 |
| peptide.induced.signaling. | | | | | | |
| pathway | | | | | | |
| X.ID.500377_1.NAME.Unwinding. | 1.863 | 1.489 | 2.331 | 5.25E−08 | 1098 | 2.19E−07 |
| of.DNA | | | | | | |
| X.ID.200199_1.NAME.p53.pathway | 1.877 | 1.493 | 2.359 | 7.10E−08 | 1098 | 2.73E−07 |
| X.ID.200173_1.NAME.Signaling. | 1.85 | 1.474 | 2.321 | 1.07E−07 | 1098 | 3.83E−07 |
| mediated.by.p38.alpha.and. | | | | | | |
| p38.beta | | | | | | |
| X.ID.200144_1.NAME.PDGFR. | 1.826 | 1.455 | 2.29 | 1.95E−07 | 1098 | 6.51E−07 |
| beta.signaling.pathway | | | | | | |
| X.ID.200098_1.NAME.Ras.signaling. | 1.817 | 1.449 | 2.279 | 2.32E−07 | 1098 | 7.24E−07 |
| in.the.CD4..TCR.pathway | | | | | | |
| X.ID.500068_1.NAME.Fanconi. | 1.725 | 1.381 | 2.156 | 1.59E−06 | 1098 | 4.69E−06 |
| Anemia.pathway | | | | | | |
| X.ID.200064_1.NAME.Wnt.signaling. | 1.678 | 1.34 | 2.103 | 6.65E−06 | 1098 | 1.85E−05 |
| network | | | | | | |
| X.ID.200090_2.NAME.mTOR. | 1.667 | 1.333 | 2.085 | 7.60E−06 | 1098 | 1.93E−05 |
| signaling.pathway | | | | | | |
| X.ID.200070_3.NAME.LKB1.signaling. | 1.675 | 1.336 | 2.1 | 7.70E−06 | 1098 | 1.93E−05 |
| events | | | | | | |
| X.ID.100084_1.NAME.hypoxia. | 1.658 | 1.324 | 2.075 | 1.02E−05 | 1098 | 2.35E−05 |
| and.p53.in.the.cardiovascular. | | | | | | |
| system | | | | | | |
| X.ID.200102_1.NAME.FoxO.family. | 1.653 | 1.322 | 2.067 | 1.03E−05 | 1098 | 2.35E−05 |
| signaling | | | | | | |
| X.ID.200189_1.NAME.Insulin. | 1.647 | 1.316 | 2.062 | 1.34E−05 | 1098 | 2.91E−05 |
| mediated.glucose.transport | | | | | | |
| X.ID.200079_1.NAME.Signaling. | 1.632 | 1.304 | 2.043 | 1.92E−05 | 1098 | 4.00E−05 |
| events.mediated.by.HDAC. | | | | | | |
| Class.I | | | | | | |
| X.ID.100159_1.NAME.cell.cycle.. | 1.628 | 1.301 | 2.038 | 2.06E−05 | 1098 | 4.11E−05 |
| g2.m.checkpoint | | | | | | |
| X.ID.100046_1.NAME.rb.tumor. | 1.615 | 1.293 | 2.016 | 2.34E−05 | 1098 | 4.32E−05 |
| suppressor.checkpoint.signaling. | | | | | | |
| in.response.to.dna.damage | | | | | | |
| X.ID.200081_2.NAME.Regulation. | 1.619 | 1.295 | 2.024 | 2.40E−05 | 1098 | 4.32E−05 |
| of.Telomerase | | | | | | |
| X.ID.500866_1.NAME.mRNA. | 1.617 | 1.293 | 2.022 | 2.50E−05 | 1098 | 4.32E−05 |
| Splicing...Major.Pathway | | | | | | |
| X.ID.100101_1.NAME.mtor.signaling. | 1.612 | 1.291 | 2.014 | 2.50E−05 | 1098 | 4.32E−05 |
| pathway | | | | | | |
| X.ID.200077_1.NAME.Circadian. | 1.612 | 1.29 | 2.013 | 2.65E−05 | 1098 | 4.42E−05 |
| rhythm.pathway | | | | | | |
| X.ID.200220_1.NAME.Notch. | 1.625 | 1.294 | 2.039 | 2.84E−05 | 1098 | 4.57E−05 |
| mediated.HES.HEY.network | | | | | | |
| X.ID.200190_1.NAME.Class.I. | 1.61 | 1.283 | 2.02 | 4.00E−05 | 1098 | 6.25E−05 |
| PI3K.signaling.events.mediated. | | | | | | |
| by.Akt | | | | | | |
| X.ID.200036_1.NAME.ATR.signaling. | 1.601 | 1.276 | 2.009 | 4.73E−05 | 1098 | 7.17E−05 |
| pathway | | | | | | |
| X.ID.500379_1.NAME.Polo.like. | 1.51 | 1.209 | 1.886 | 2.84E−04 | 1098 | 0.0004176 |
| kinase.mediated.events | | | | | | |
| X.ID.200128_1.NAME.Syndecan. | 1.51 | 1.208 | 1.887 | 2.96E−04 | 1098 | 0.0004229 |
| 4.mediated.signaling.events | | | | | | |
| X.ID.100122_1.NAME.intrinsic. | 1.495 | 1.195 | 1.871 | 0.0004397 | 1098 | 0.0006107 |
| prothrombin.activation.pathway | | | | | | |
| X.ID.500945_1.NAME.Removal. | 1.474 | 1.183 | 1.838 | 5.49E−04 | 1098 | 0.0007417 |
| of.DNA.patch.containing. | | | | | | |
| abasic.residue | | | | | | |
| X.ID.200166_2.NAME.Caspase. | 1.476 | 1.181 | 1.845 | 6.13E−04 | 1098 | 0.0008066 |
| cascade.in.apoptosis | | | | | | |
| X.ID.200152_1.NAME.p38.signaling. | 1.475 | 1.18 | 1.844 | 0.0006397 | 1098 | 0.0008201 |
| mediated.by.MAPKAP.kinases | | | | | | |
| X.ID.200129_1.NAME.ATF.2. | 1.437 | 1.153 | 1.792 | 0.0012535 | 1098 | 0.0015669 |
| transcription.factor.network | | | | | | |
| X.ID.200048_1.NAME.Calcineurin. | 1.439 | 1.152 | 1.797 | 0.0013493 | 1098 | 0.0016455 |
| regulated.NFAT.dependent. | | | | | | |
| transcription.in.lymphocytes | | | | | | |
| X.ID.500652_1.NAME.Generic. | 1.408 | 1.13 | 1.755 | 2.26E−03 | 1098 | 0.0026939 |
| Transcription.Pathway | | | | | | |
| X.ID.100144_1.NAME.hiv.1.nef.. | 1.373 | 1.099 | 1.716 | 5.27E−03 | 1098 | 0.0061252 |
| negative.effector.of.fas.and.tnf | | | | | | |
| X.ID.200132_1.NAME.AP.1.transcription. | 1.356 | 1.087 | 1.691 | 6.85E−03 | 1098 | 0.0077826 |
| factor.network | | | | | | |
| X.ID.200126_2.NAME.ErbB1. | 1.356 | 1.085 | 1.694 | 0.0073698 | 1098 | 0.0081886 |
| downstream.signaling | | | | | | |
| X.ID.200208_2.NAME.Downstream. | 1.336 | 1.071 | 1.666 | 1.03E−02 | 1098 | 0.0112107 |
| signaling.in.naive.CD8..T.cells | | | | | | |
| X.ID.100085_1.NAME.p38.mapk. | 1.329 | 1.065 | 1.659 | 0.0117017 | 1098 | 0.0124487 |
| signaling.pathway | | | | | | |
| X.ID.100218_1.NAME.caspase. | 1.322 | 1.06 | 1.649 | 1.33E−02 | 1098 | 0.0138185 |
| cascade.in.apoptosis | | | | | | |
| X.ID.200076_2.NAME.FAS..CD95.. | 1.276 | 1.022 | 1.593 | 3.16E−02 | 1098 | 0.0322634 |
| signaling.pathway | | | | | | |
| X.ID.500755_1.NAME.Nef.and. | 1.213 | 0.973 | 1.513 | 0.0860009 | 1098 | 0.0860009 |
| signal.transduction | | | | | | |

TABLE 14
Breast cancer Model E. Hazard ratios (95% CI, p values, size of the validation cohort and q values)
of patients' MDS based classification. A univariate Cox proportional hazards model was fit to each of the top ranked
subnetwork markers (n_{Breast} = 50, n_{Colon} = 75, n_{NSCLC} = 25 and n_{Ovarian} =
50) and subsequently applied to predict patient risk score in the validation cohort. The survival
differences between the predicted groups were assessed using Kaplan-Meier analysis.

| | | 95% CI | 95% CI | | | |
|---|---|---|---|---|---|---|
| Subnetwork module | HR | lower | upper | P | n | Q |
| X.ID.200003_1.NAME.Fc.epsilon.receptor. | 1.418 | 1.136 | 1.77 | 2.01E−03 | 1098 | 3.86E−02 |
| I.signaling.in.mast.cells | | | | | | |
| X.ID.200178_1.NAME.Calcium.signaling. | 1.409 | 1.132 | 1.755 | 2.17E−03 | 1098 | 3.86E−02 |
| in.the.CD4..TCR.pathway | | | | | | |
| X.ID.200040_1.NAME.Signaling.events. | 1.419 | 1.133 | 1.776 | 2.32E−03 | 1098 | 3.86E−02 |
| mediated.by.PTP1B | | | | | | |
| X.ID.200048_1.NAME.Calcineurin.regulated. | 1.364 | 1.093 | 1.702 | 5.98E−03 | 1098 | 6.01E−02 |
| NFAT.dependent.transcription.in.lymphocytes | | | | | | |
| X.ID.200011_1.NAME.Aurora.B.signaling | 1.365 | 1.093 | 1.704 | 6.01E−03 | 1098 | 6.01E−02 |
| X.ID.200175_6.NAME.Signaling.events. | 0.74 | 0.593 | 0.923 | 7.69E−03 | 1098 | 6.41E−02 |
| mediated.by.Stem.cell.factor.receptor.. | | | | | | |
| c.Kit. | | | | | | |
| X.ID.100152_1.NAME.inactivation.of. | 1.235 | 0.991 | 1.538 | 6.02E−02 | 1098 | 3.78E−01 |
| gsk3.by.akt.causes.accumulation.of.b. | | | | | | |
| catenin.in.alveolar.macrophages | | | | | | |
| X.ID.500866_3.NAME.mRNA.Splicing... | 0.815 | 0.654 | 1.014 | 6.68E−02 | 1098 | 3.78E−01 |
| Major.Pathway | | | | | | |
| X.ID.100113_1.NAME.mapkinase.signaling. | 1.223 | 0.981 | 1.523 | 7.33E−02 | 1098 | 3.78E−01 |
| pathway | | | | | | |
| X.ID.100077_1.NAME.pdgf.signaling.pathway | 1.218 | 0.978 | 1.517 | 7.79E−02 | 1098 | 3.78E−01 |
| X.ID.200097_1.NAME.PLK1.signaling. | 1.215 | 0.975 | 1.513 | 8.31E−02 | 1098 | 3.78E−01 |
| events | | | | | | |
| X.ID.200168_1.NAME.CXCR3.mediated. | 1.211 | 0.969 | 1.514 | 9.24E−02 | 1098 | 3.85E−01 |
| signaling.events | | | | | | |
| X.ID.200187_1.NAME.Aurora.A.signaling | 1.191 | 0.956 | 1.485 | 1.19E−01 | 1098 | 4.52E−01 |
| X.ID.200102_1.NAME.FoxO.family.signaling | 1.189 | 0.952 | 1.484 | 1.27E−01 | 1098 | 4.52E−01 |
| X.ID.100218_1.NAME.caspase.cascade. | 0.848 | 0.681 | 1.056 | 1.42E−01 | 1098 | 4.73E−01 |
| in.apoptosis | | | | | | |
| X.ID.100026_2.NAME.tnf.stress.related. | 0.862 | 0.691 | 1.075 | 1.87E−01 | 1098 | 5.84E−01 |
| signaling | | | | | | |
| X.ID.200158_1.NAME.Retinoic.acid. | 0.868 | 0.697 | 1.081 | 2.07E−01 | 1098 | 5.96E−01 |
| receptors.mediated.signaling | | | | | | |
| X.ID.100245_2.NAME.akt.signaling.pathway | 1.146 | 0.92 | 1.426 | 2.24E−01 | 1098 | 5.96E−01 |
| X.ID.200081_2.NAME.Regulation.of.Telomerase | 1.146 | 0.919 | 1.428 | 2.27E−01 | 1098 | 5.96E−01 |
| X.ID.200022_1.NAME.Signaling.events. | 0.88 | 0.706 | 1.095 | 2.52E−01 | 1098 | 6.27E−01 |
| mediated.by.HDAC.Class.II | | | | | | |
| X.ID.100008_1.NAME.ucalpain.and.friends. | 1.133 | 0.91 | 1.411 | 2.63E−01 | 1098 | 6.27E−01 |
| in.cell.spread | | | | | | |
| X.ID.100002_1.NAME.wnt.signaling.pathway | 1.11 | 0.891 | 1.382 | 3.51E−01 | 1098 | 7.71E−01 |
| X.ID.200122_1.NAME.Integrins.in.angiogenesis | 0.902 | 0.724 | 1.123 | 3.55E−01 | 1098 | 7.71E−01 |
| X.ID.100250_1.NAME.hemoglobins.chaperone | 0.907 | 0.729 | 1.13 | 3.84E−01 | 1098 | 7.91E−01 |
| X.ID.100144_1.NAME.hiv.1.nef..negative. | 1.1 | 0.883 | 1.369 | 3.95E−01 | 1098 | 7.91E−01 |
| effector.of.fas.and.tnf | | | | | | |
| X.ID.200199_1.NAME.p53.pathway | 0.917 | 0.736 | 1.142 | 4.38E−01 | 1098 | 8.42E−01 |
| X.ID.200043_1.NAME.IL12.mediated.signaling. | 1.079 | 0.866 | 1.343 | 4.97E−01 | 1098 | 9.21E−01 |
| events | | | | | | |
| X.ID.100132_1.NAME.signal.transduction. | 0.933 | 0.749 | 1.162 | 5.34E−01 | 1098 | 9.50E−01 |
| through.il1r | | | | | | |
| X.ID.100149_1.NAME.human.cytomegalovirus. | 0.939 | 0.754 | 1.169 | 5.71E−01 | 1098 | 9.50E−01 |
| and.map.kinase.pathways | | | | | | |
| X.ID.500652_1.NAME.Generic.Transcription. | 1.065 | 0.853 | 1.331 | 5.77E−01 | 1098 | 9.50E−01 |
| Pathway | | | | | | |
| X.ID.200061_2.NAME.Presenilin.action. | 1.061 | 0.85 | 1.325 | 6.01E−01 | 1098 | 9.50E−01 |
| in.Notch.and.Wnt.signaling | | | | | | |
| X.ID.500655_1.NAME.Processing.of.Capped. | 1.059 | 0.849 | 1.321 | 6.10E−01 | 1098 | 9.50E−01 |
| Intron.Containing.Pre.mRNA | | | | | | |
| X.ID.200081_1.NAME.Regulation.of.Telomerase | 0.95 | 0.762 | 1.184 | 6.47E−01 | 1098 | 9.50E−01 |
| X.ID.100132_2.NAME.signal.transduction. | 0.952 | 0.764 | 1.185 | 6.58E−01 | 1098 | 0.95018229 |
| through.il1r | | | | | | |
| X.ID.100119_1.NAME.keratinocyte.differentiation | 0.953 | 0.766 | 1.187 | 6.70E−01 | 1098 | 0.95018229 |
| X.ID.200079_1.NAME.Signaling.events. | 1.042 | 0.837 | 1.297 | 0.71227 | 1098 | 0.95018229 |
| mediated.by.HDAC.Class.I | | | | | | |
| X.ID.200165_1.NAME.Hedgehog.signaling. | 1.042 | 0.836 | 1.298 | 7.14E−01 | 1098 | 0.95018229 |
| events.mediated.by.Gli.proteins | | | | | | |
| X.ID.200215_2.NAME.Regulation.of.retinoblastoma. | 1.039 | 0.833 | 1.294 | 7.35E−01 | 1098 | 0.95018229 |
| protein | | | | | | |
| X.ID.200153_1.NAME.ErbB.receptor.signaling. | 1.035 | 0.831 | 1.289 | 0.75675 | 1098 | 0.95018229 |
| network | | | | | | |
| X.ID.500128_1.NAME.Insulin.Synthesis. | 1.035 | 0.83 | 1.291 | 0.76015 | 1098 | 0.95018229 |
| and.Processing | | | | | | |
| X.ID.200019_2.NAME.Noncanonical.Wnt. | 1.029 | 0.826 | 1.281 | 0.79836 | 1098 | 0.96202964 |
| signaling.pathway | | | | | | |
| X.ID.100029_1.NAME.sprouty.regulation. | 1.026 | 0.824 | 1.278 | 8.18E−01 | 1098 | 0.96202964 |
| of.tyrosine.kinase.signals | | | | | | |
| X.ID.500866_1.NAME.mRNA.Splicing... | 1.021 | 0.819 | 1.275 | 8.51E−01 | 1098 | 0.96202964 |
| Major.Pathway | | | | | | |
| X.ID.100123_1.NAME.integrin.signaling. | 1.019 | 0.819 | 1.269 | 8.64E−01 | 1098 | 0.96202964 |
| pathway | | | | | | |
| X.ID.100226_1.NAME.bioactive.peptide. | 0.985 | 0.791 | 1.226 | 0.88936 | 1098 | 0.96202964 |
| induced.signaling.pathway | | | | | | |
| X.ID.200112_1.NAME.IL2.signaling.events. | 0.986 | 0.792 | 1.227 | 8.98E−01 | 1098 | 0.96202964 |
| mediated.by.PI3K | | | | | | |
| X.ID.100116_4.NAME.lissencephaly.gene.. | 0.987 | 0.793 | 1.229 | 0.90726 | 1098 | 0.96202964 |
| lis1..in.neuronal.migration.and.development | | | | | | |
| X.ID.200206_1.NAME.Trk.receptor.signaling. | 1.011 | 0.812 | 1.259 | 9.24E−01 | 1098 | 0.96202964 |
| mediated.by.the.MAPK.pathway | | | | | | |
| X.ID.500128_2.NAME.Insulin.Synthesis. | 1.007 | 0.806 | 1.26 | 9.49E−01 | 1098 | 0.96821648 |
| and.Processing | | | | | | |
| X.ID.200166_2.NAME.Caspase.cascade. | 1 | 0.803 | 1.245 | 0.99904 | 1098 | 0.9990366 |
| in.apoptosis | | | | | | |

TABLE 15
Colon cancer Model N + E. Hazard ratios (95% CI, p values, size of the validation cohort and q values)
of patients' MDS based classification. A univariate Cox proportional hazards model was fit to each of the top ranked subnetwork
markers (n_{Breast} = 50, n_{Colon} = 75, n_{NSCLC} = 25 and n_{Ovarian} = 50) and subsequently
applied to predict patient risk score in the validation cohort. The survival differences between the
predicted groups were assessed using Kaplan-Meier analysis.

| | | 95% CI | 95% CI | | | |
|---|---|---|---|---|---|---|
| Subnetwork module | HR | lower | upper | P | n | Q |
| X.ID.200173_1.NAME.Signaling.mediated.by.p38.alpha. | 2.109 | 1.368 | 3.25 | 0.000724196 | 312 | 0.054314697 |
| and.p38.beta | | | | | | |
| X.ID.100062_2.NAME.prion.pathway | 1.874 | 1.217 | 2.886 | 0.004368969 | 312 | 0.086869055 |
| X.ID.200122_1.NAME.Integrins.in.angiogenesis | 1.83 | 1.192 | 2.811 | 0.005747417 | 312 | 0.086869055 |
| X.ID.100094_1.NAME.actions.of.nitric.oxide.in.the. | 1.834 | 1.189 | 2.83 | 0.006076721 | 312 | 0.086869055 |
| heart | | | | | | |
| X.ID.100137_1.NAME.skeletal.muscle.hypertrophy. | 1.814 | 1.181 | 2.786 | 0.006542442 | 312 | 0.086869055 |
| is.regulated.via.akt.mtor.pathway | | | | | | |
| X.ID.100218_1.NAME.caspase.cascade.in.apoptosis | 1.855 | 1.184 | 2.905 | 0.006949524 | 312 | 0.086869055 |
| X.ID.100164_1.NAME.fibrinolysis.pathway | 1.757 | 1.15 | 2.685 | 0.009167197 | 312 | 0.096217813 |
| X.ID.100113_1.NAME.mapkinase.signaling.pathway | 1.771 | 1.145 | 2.741 | 0.010263233 | 312 | 0.096217813 |
| X.ID.200185_1.NAME.Syndecan.2.mediated.signaling. | 1.701 | 1.095 | 2.641 | 0.018080251 | 312 | 0.150668757 |
| events | | | | | | |
| X.ID.100144_1.NAME.hiv.1.nef..negative.effector.of. | 1.623 | 1.049 | 2.51 | 0.029653442 | 312 | 0.222400818 |
| fas.and.tnf | | | | | | |
| X.ID.100056_1.NAME.rac1.cell.motility.signaling.pathway | 1.589 | 1.035 | 2.441 | 0.034253044 | 312 | 0.233543481 |
| X.ID.200079_1.NAME.Signaling.events.mediated.by. | 1.532 | 1.012 | 2.32 | 0.043909118 | 312 | 0.243525474 |
| HDAC.Class.I | | | | | | |
| X.ID.100122_1.NAME.intrinsic.prothrombin.activation. | 1.555 | 1.008 | 2.398 | 0.045727865 | 312 | 0.243525474 |
| pathway | | | | | | |
| X.ID.100085_1.NAME.p38.mapk.signaling.pathway | 1.542 | 1.003 | 2.373 | 0.04866992 | 312 | 0.243525474 |
| X.ID.200216_1.NAME.Signaling.events.mediated.by. | 1.526 | 1.002 | 2.322 | 0.048705095 | 312 | 0.243525474 |
| focal.adhesion.kinase | | | | | | |
| X.ID.100072_1.NAME.platelet.amyloid.precursor. | 1.519 | 0.992 | 2.325 | 0.054295499 | 312 | 0.252590222 |
| protein.pathway | | | | | | |
| X.ID.200199_1.NAME.p53.pathway | 1.509 | 0.987 | 2.306 | 0.057253784 | 312 | 0.252590222 |
| X.ID.200017_1.NAME.p38.MAPK.signaling.pathway | 0.675 | 0.441 | 1.034 | 0.070847006 | 312 | 0.295195857 |
| X.ID.200139_2.NAME.BMP.receptor.signaling | 1.439 | 0.945 | 2.192 | 0.089638591 | 312 | 0.353836542 |
| X.ID.500455_1.NAME.ERK.MAPK.targets | 1.43 | 0.939 | 2.177 | 0.095194471 | 312 | 0.356979266 |
| X.ID.200139_1.NAME.BMP.receptor.signaling | 1.427 | 0.934 | 2.18 | 0.100477363 | 312 | 0.358847723 |
| X.ID.500655_1.NAME.Processing.of.Capped.Intron. | 0.708 | 0.465 | 1.078 | 0.107758028 | 312 | 0.367356914 |
| Containing.Pre.mRNA | | | | | | |
| X.ID.200011_1.NAME.Aurora.B.signaling | 1.427 | 0.919 | 2.216 | 0.113653061 | 312 | 0.370607808 |
| X.ID.100084_1.NAME.hypoxia.and.p53.in.the.cardiovascular. | 1.387 | 0.915 | 2.102 | 0.122682838 | 312 | 0.372540666 |
| system | | | | | | |
| X.ID.100171_1.NAME.role.of.erk5.in.neuronal.survival. | 1.392 | 0.913 | 2.124 | 0.124729629 | 312 | 0.372540666 |
| pathway | | | | | | |
| X.ID.200183_2.NAME.a6b1.and.a6b4.Integrin.signaling | 0.727 | 0.48 | 1.103 | 0.133649024 | 312 | 0.372540666 |
| X.ID.500128_1.NAME.Insulin.Synthesis.and.Processing | 0.726 | 0.478 | 1.104 | 0.13411464 | 312 | 0.372540666 |
| X.ID.100022_1.NAME.t.cell.receptor.signaling.pathway | 1.356 | 0.889 | 2.068 | 0.156947874 | 312 | 0.42039609 |
| X.ID.100184_1.NAME.erk.and.pi.3.kinase.are.necessary. | 1.347 | 0.872 | 2.083 | 0.179562904 | 312 | 0.452552269 |
| for.collagen.binding.in.corneal.epithelia | | | | | | |
| X.ID.200187_1.NAME.Aurora.A.signaling | 1.333 | 0.873 | 2.037 | 0.1830561 | 312 | 0.452552269 |
| X.ID.200175_6.NAME.Signaling.events.mediated.by. | 0.757 | 0.499 | 1.149 | 0.190801554 | 312 | 0.452552269 |
| Stem.cell.factor.receptor..c.Kit. | | | | | | |
| X.ID.200040_1.NAME.Signaling.events.mediated.by. | 1.318 | 0.869 | 2 | 0.193693813 | 312 | 0.452552269 |
| PTP1B | | | | | | |
| X.ID.100041_1.NAME.rho.cell.motility.signaling.pathway | 1.316 | 0.863 | 2.007 | 0.201513288 | 312 | 0.452552269 |
| X.ID.100123_1.NAME.integrin.signaling.pathway | 1.316 | 0.848 | 2.045 | 0.220900343 | 312 | 0.452552269 |
| X.ID.200175_2.NAME.Signaling.events.mediated.by. | 0.771 | 0.508 | 1.17 | 0.221227954 | 312 | 0.452552269 |
| Stem.cell.factor.receptor..c.Kit. | | | | | | |
| X.ID.500866_1.NAME.mRNA.Splicing...Major.Pathway | 0.765 | 0.498 | 1.176 | 0.22264883 | 312 | 0.452552269 |
| X.ID.100047_1.NAME.ras.signaling.pathway | 0.774 | 0.511 | 1.173 | 0.227207044 | 312 | 0.452552269 |
| X.ID.200024_1.NAME.Signaling.events.mediated.by. | 1.294 | 0.847 | 1.976 | 0.233796553 | 312 | 0.452552269 |
| HDAC.Class.III | | | | | | |
| X.ID.200085_1.NAME.Role.of.Calcineurin.dependent. | 1.283 | 0.848 | 1.941 | 0.238500228 | 312 | 0.452552269 |
| NFAT.signaling.in.lymphocytes | | | | | | |
| X.ID.200127_2.NAME.Lissencephaly.gene..LIS1..in. | 1.287 | 0.844 | 1.962 | 0.24136121 | 312 | 0.452552269 |
| neuronal.migration.and.development | | | | | | |
| X.ID.100106_1.NAME.role.of.mitochondria.in.apoptotic. | 1.266 | 0.837 | 1.915 | 0.263315566 | 312 | 0.481674815 |
| signaling | | | | | | |
| X.ID.200064_1.NAME.Wnt.signaling.network | 1.262 | 0.831 | 1.915 | 0.274911012 | 312 | 0.490912521 |
| X.ID.200134_1.NAME.Urokinase.type.plasminogen. | 0.808 | 0.534 | 1.222 | 0.312687115 | 312 | 0.545384503 |
| activator..uPA..and.uPAR.mediated.signaling | | | | | | |
| X.ID.100119_1.NAME.keratinocyte.differentiation | 1.233 | 0.808 | 1.88 | 0.331395693 | 312 | 0.564879023 |
| X.ID.200166_2.NAME.Caspase.cascade.in.apoptosis | 1.232 | 0.8 | 1.899 | 0.343486159 | 312 | 0.572476931 |
| X.ID.200171_1.NAME.Regulation.of.cytoplasmic.and. | 0.821 | 0.542 | 1.245 | 0.352631992 | 312 | 0.574943466 |
| nuclear.SMAD2.3.signaling | | | | | | |
| X.ID.100111_1.NAME.mcalpain.and.friends.in.cell. | 1.213 | 0.801 | 1.837 | 0.362721833 | 312 | 0.578811436 |
| motility | | | | | | |
| X.ID.200190_1.NAME.Class.I.PI3K.signaling.events. | 1.193 | 0.787 | 1.809 | 0.405365009 | 312 | 0.622369202 |
| mediated.by.Akt | | | | | | |
| X.ID.100162_1.NAME.fmlp.induced.chemokine.gene. | 1.19 | 0.784 | 1.805 | 0.414630968 | 312 | 0.622369202 |
| expression.in.hmc.1.cells | | | | | | |
| X.ID.200102_1.NAME.FoxO.family.signaling | 1.188 | 0.785 | 1.797 | 0.414912801 | 312 | 0.622369202 |
| X.ID.200126_2.NAME.ErbB1.downstream.signaling | 1.174 | 0.771 | 1.787 | 0.45597355 | 312 | 0.670549338 |
| X.ID.200144_1.NAME.PDGFR.beta.signaling.pathway | 0.864 | 0.57 | 1.31 | 0.492294052 | 312 | 0.710039497 |
| X.ID.200128_1.NAME.Syndecan.4.mediated.signaling. | 1.146 | 0.755 | 1.739 | 0.521870209 | 312 | 0.724764874 |
| events | | | | | | |
| X.ID.100095_2.NAME.ras.independent.pathway.in. | 0.878 | 0.58 | 1.328 | 0.537078076 | 312 | 0.724764874 |
| nk.cell.mediated.cytotoxicity | | | | | | |
| X.ID.100008_1.NAME.ucalpain.and.friends.in.cell.spread | 1.139 | 0.751 | 1.729 | 0.540394118 | 312 | 0.724764874 |
| X.ID.100032_1.NAME.map.kinase.inactivation.of.smrt. | 1.134 | 0.748 | 1.719 | 0.553674516 | 312 | 0.724764874 |
| corepressor | | | | | | |
| X.ID.100233_1.NAME.regulation.of.bad.phosphorylation | 0.884 | 0.584 | 1.337 | 0.558077874 | 312 | 0.724764874 |
| X.ID.200026_3.NAME.TCR.signaling.in.naive.CD4..T.cells | 0.883 | 0.581 | 1.343 | 0.560484836 | 312 | 0.724764874 |
| X.ID.200164_1.NAME.Internalization.of.ErbB1 | 0.887 | 0.585 | 1.345 | 0.573671689 | 312 | 0.729243673 |
| X.ID.500652_1.NAME.Generic.Transcription.Pathway | 0.892 | 0.589 | 1.35 | 0.587827659 | 312 | 0.734784574 |
| X.ID.200006_1.NAME.Signaling.events.mediated.by. | 0.894 | 0.589 | 1.358 | 0.599943062 | 312 | 0.737634913 |
| PRL | | | | | | |
| X.ID.500799_1.NAME.Hormone.sensitive.lipase..HSL.. | 1.115 | 0.732 | 1.697 | 0.611847771 | 312 | 0.740138432 |
| mediated.triacylglycerol.hydrolysis | | | | | | |
| X.ID.200012_3.NAME.LPA.receptor.mediated.events | 1.108 | 0.732 | 1.677 | 0.627738368 | 312 | 0.746142759 |
| X.ID.200090_1.NAME.mTOR.signaling.pathway | 1.105 | 0.73 | 1.673 | 0.637779129 | 312 | 0.746142759 |
| X.ID.100178_1.NAME.regulation.of.eif.4e.and.p70s6. | 1.101 | 0.728 | 1.666 | 0.649068778 | 312 | 0.746142759 |
| kinase | | | | | | |
| X.ID.200165_1.NAME.Hedgehog.signaling.events. | 1.099 | 0.725 | 1.666 | 0.656605628 | 312 | 0.746142759 |
| mediated.by.Gli.proteins | | | | | | |
| X.ID.500575_2.NAME.RNA.Polymerase.I.Transcription. | 1.091 | 0.718 | 1.658 | 0.683078041 | 312 | 0.764639599 |
| Initiation | | | | | | |
| X.ID.100132_1.NAME.signal.transduction.through.il1r | 1.07 | 0.708 | 1.618 | 0.747857299 | 312 | 0.82117202 |
| X.ID.100083_1.NAME.p53.signaling.pathway | 0.936 | 0.619 | 1.416 | 0.755478258 | 312 | 0.82117202 |
| X.ID.200070_3.NAME.LKB1.signaling.events | 0.949 | 0.627 | 1.435 | 0.802474066 | 312 | 0.859793642 |
| X.ID.200189_1.NAME.Insulin.mediated.glucose.transport | 1.039 | 0.685 | 1.578 | 0.855631545 | 312 | 0.903836139 |
| X.ID.200070_1.NAME.LKB1.signaling.events | 1.035 | 0.682 | 1.571 | 0.870146167 | 312 | 0.906402257 |
| X.ID.200129_1.NAME.ATF.2.transcription.factor.network | 1.019 | 0.672 | 1.545 | 0.929765995 | 312 | 0.948230282 |
| X.ID.200114_2.NAME.Direct.p53.effectors | 1.017 | 0.671 | 1.542 | 0.935587212 | 312 | 0.948230282 |
| X.ID.200206_1.NAME.Trk.receptor.signaling.mediated. | 1.008 | 0.663 | 1.533 | 0.969574433 | 312 | 0.969574433 |
| by.the.MAPK.pathway | | | | | | |

TABLE 15
Colon cancer Model N. Hazard ratios (95% CI, p values, size of the validation cohort and q values)
of patients' MDS based classification. A univariate Cox proportional hazards model was fit to each of the top ranked
subnetwork markers (n_{Breast} = 50, n_{Colon} = 75, n_{NSCLC} = 25 and n_{Ovarian} = 50) and
subsequently applied to predict patient risk score in the validation cohort. The survival differences
between the predicted groups were assessed using Kaplan-Meier analysis.

| | | 95% CI | 95% CI | | | |
|---|---|---|---|---|---|---|
| Subnetwork module | HR | lower | upper | P | n | Q |
| X.ID.200173_1.NAME.Signaling.mediated.by. | 2.964 | 1.831 | 4.798 | 9.83875E−06 | 312 | 0.000737906 |
| p38.alpha.and.p38.beta | | | | | | |
| X.ID.100164_1.NAME.fibrinolysis.pathway | 2.614 | 1.636 | 4.176 | 5.829E−05 | 312 | 0.002185874 |
| X.ID.100072_1.NAME.platelet.amyloid.precursor. | 2.499 | 1.564 | 3.992 | 0.000126589 | 312 | 0.003164715 |
| protein.pathway | | | | | | |
| X.ID.100113_1.NAME.mapkinase.signaling.pathway | 2.435 | 1.514 | 3.918 | 0.000242855 | 312 | 0.003888753 |
| X.ID.200175_4.NAME.Signaling.events.mediated. | 2.343 | 1.484 | 3.7 | 0.00025925 | 312 | 0.003888753 |
| by.Stem.cell.factor.receptor..c.Kit. | | | | | | |
| X.ID.500123_1.NAME.Cell.extracellular.matrix. | 2.207 | 1.41 | 3.454 | 0.000532642 | 312 | 0.006658023 |
| interactions | | | | | | |
| X.ID.100218_1.NAME.caspase.cascade.in.apoptosis | 2.197 | 1.39 | 3.473 | 0.000755965 | 312 | 0.008099628 |
| X.ID.100094_1.NAME.actions.of.nitric.oxide.in. | 2.029 | 1.311 | 3.14 | 0.001487792 | 312 | 0.013948047 |
| the.heart | | | | | | |
| X.ID.100122_1.NAME.intrinsic.prothrombin. | 1.989 | 1.275 | 3.103 | 0.002452958 | 312 | 0.020441318 |
| activation.pathway | | | | | | |
| X.ID.200122_1.NAME.Integrins.in.angiogenesis | 1.927 | 1.251 | 2.968 | 0.002926279 | 312 | 0.020799725 |
| X.ID.200171_1.NAME.Regulation.of.cytoplasmic. | 1.906 | 1.244 | 2.921 | 0.003050626 | 312 | 0.020799725 |
| and.nuclear.SMAD2.3.signaling | | | | | | |
| X.ID.100129_1.NAME.il.2.receptor.beta.chain. | 1.94 | 1.236 | 3.046 | 0.003977901 | 312 | 0.023419134 |
| in.t.cell.activation | | | | | | |
| X.ID.200012_2.NAME.LPA.receptor.mediated. | 1.867 | 1.22 | 2.859 | 0.004059317 | 312 | 0.023419134 |
| events | | | | | | |
| X.ID.200061_1.NAME.Presenilin.action.in.Notch. | 1.914 | 1.224 | 2.993 | 0.004397436 | 312 | 0.023557695 |
| and.Wnt.signaling | | | | | | |
| X.ID.100171_1.NAME.role.of.erk5.in.neuronal. | 1.818 | 1.176 | 2.811 | 0.00715273 | 312 | 0.035763649 |
| survival.pathway | | | | | | |
| X.ID.100108_1.NAME.melanocyte.development. | 1.816 | 1.171 | 2.817 | 0.007690845 | 312 | 0.035766463 |
| and.pigmentation.pathway | | | | | | |
| X.ID.200040_1.NAME.Signaling.events.mediated. | 1.831 | 1.17 | 2.866 | 0.008107065 | 312 | 0.035766463 |
| by.PTP1B | | | | | | |
| X.ID.200081_2.NAME.Regulation.of.Telomerase | 1.732 | 1.133 | 2.647 | 0.011169272 | 312 | 0.043184849 |
| X.ID.200185_1.NAME.Syndecan.2.mediated. | 1.758 | 1.135 | 2.721 | 0.011443358 | 312 | 0.043184849 |
| signaling.events | | | | | | |
| X.ID.200064_1.NAME.Wnt.signaling.network | 1.745 | 1.133 | 2.687 | 0.01151596 | 312 | 0.043184849 |
| X.ID.100137_1.NAME.skeletal.muscle.hypertrophy. | 1.696 | 1.115 | 2.578 | 0.013463278 | 312 | 0.04590462 |
| is.regulated.via.akt.mtor.pathway | | | | | | |
| X.ID.500866_1.NAME.mRNA.Splicing...Major. | 1.691 | 1.115 | 2.565 | 0.013465355 | 312 | 0.04590462 |
| Pathway | | | | | | |
| X.ID.100022_1.NAME.t.cell.receptor.signaling. | 1.731 | 1.115 | 2.687 | 0.014539819 | 312 | 0.047412452 |
| pathway | | | | | | |
| X.ID.200011_1.NAME.Aurora.B.signaling | 1.666 | 1.09 | 2.545 | 0.018382058 | 312 | 0.05474464 |
| X.ID.100062_2.NAME.prion.pathway | 1.646 | 1.086 | 2.496 | 0.018840234 | 312 | 0.05474464 |
| X.ID.100162_1.NAME.fmlp.induced.chemokine. | 1.662 | 1.087 | 2.541 | 0.018978142 | 312 | 0.05474464 |
| gene.expression.in.hmc.1.cells | | | | | | |
| X.ID.200127_2.NAME.Lissencephaly.gene..LIS1. | 1.652 | 1.08 | 2.526 | 0.020522395 | 312 | 0.056342735 |
| in.neuronal.migration.and.development | | | | | | |
| X.ID.200216_1.NAME.Signaling.events.mediated. | 1.665 | 1.08 | 2.568 | 0.021034621 | 312 | 0.056342735 |
| by.focal.adhesion.kinase | | | | | | |
| X.ID.200206_1.NAME.Trk.receptor.signaling. | 1.647 | 1.075 | 2.524 | 0.021787075 | 312 | 0.056345883 |
| mediated.by.the.MAPK.pathway | | | | | | |
| X.ID.500406_1.NAME.Chemokine.receptors. | 1.649 | 1.07 | 2.541 | 0.023339502 | 312 | 0.058348754 |
| bind.chemokines | | | | | | |
| X.ID.200166_2.NAME.Caspase.cascade.in.apoptosis | 1.676 | 1.061 | 2.648 | 0.026890143 | 312 | 0.065056797 |
| X.ID.100184_1.NAME.erk.and.pi.3.kinase.are. | 1.608 | 1.047 | 2.471 | 0.03016214 | 312 | 0.070692517 |
| necessary.for.collagen.binding.in.corneal.epithelia | | | | | | |
| X.ID.200109_1.NAME.Sumoylation.by.RanBP2. | 1.616 | 1.038 | 2.515 | 0.033605359 | 312 | 0.076375815 |
| regulates.transcriptional.repression | | | | | | |
| X.ID.500652_1.NAME.Generic.Transcription. | 1.594 | 1.028 | 2.472 | 0.037338971 | 312 | 0.080712058 |
| Pathway | | | | | | |
| X.ID.100085_1.NAME.p38.mapk.signaling.pathway | 1.586 | 1.027 | 2.45 | 0.037665627 | 312 | 0.080712058 |
| X.ID.200079_1.NAME.Signaling.events.mediated. | 1.519 | 0.999 | 2.31 | 0.050342029 | 312 | 0.104879227 |
| by.HDAC.Class.I | | | | | | |
| X.ID.100168_1.NAME.extrinsic.prothrombin. | 1.515 | 0.996 | 2.305 | 0.052481053 | 312 | 0.106380513 |
| activation.pathway | | | | | | |
| X.ID.200139_2.NAME.BMP.receptor.signaling | 1.482 | 0.975 | 2.252 | 0.065516134 | 312 | 0.128499202 |
| X.ID.100111_1.NAME.mcalpain.and.friends.in. | 1.515 | 0.972 | 2.363 | 0.066819585 | 312 | 0.128499202 |
| cell.motility | | | | | | |
| X.ID.200070_1.NAME.LKB1.signaling.events | 1.449 | 0.948 | 2.214 | 0.08643956 | 312 | 0.162074174 |
| X.ID.100189_1.NAME.induction.of.apoptosis. | 1.42 | 0.928 | 2.173 | 0.106510872 | 312 | 0.19483696 |
| through.dr3.and.dr4.5.death.receptors | | | | | | |
| X.ID.100018_2.NAME.trefoil.factors.initiate.mucosal. | 1.391 | 0.918 | 2.109 | 0.119679116 | 312 | 0.21084113 |
| healing | | | | | | |
| X.ID.100008_1.NAME.ucalpain.and.friends.in. | 1.401 | 0.915 | 2.145 | 0.120882248 | 312 | 0.21084113 |
| cell.spread | | | | | | |
| X.ID.100106_1.NAME.role.of.mitochondria.in. | 1.378 | 0.909 | 2.089 | 0.130423674 | 312 | 0.222233832 |
| apoptotic.signaling | | | | | | |
| X.ID.200090_1.NAME.mTOR.signaling.pathway | 1.382 | 0.906 | 2.107 | 0.133340299 | 312 | 0.222233832 |
| X.ID.100095_2.NAME.ras.independent.pathway. | 1.356 | 0.889 | 2.067 | 0.157516268 | 312 | 0.256820003 |
| in.nk.cell.mediated.cytotoxicity | | | | | | |
| X.ID.200199_1.NAME.p53.pathway | 1.349 | 0.881 | 2.067 | 0.168695055 | 312 | 0.269194237 |
| X.ID.200126_2.NAME.ErbB1.downstream.signaling | 1.32 | 0.862 | 2.021 | 0.201979776 | 312 | 0.3155934 |
| X.ID.100041_1.NAME.rho.cell.motility.signaling. | 1.285 | 0.843 | 1.959 | 0.244134135 | 312 | 0.373674696 |
| pathway | | | | | | |
| X.ID.200128_1.NAME.Syndecan.4.mediated. | 1.272 | 0.836 | 1.937 | 0.261092032 | 312 | 0.391638049 |
| signaling.events | | | | | | |
| X.ID.100056_1.NAME.rac1.cell.motility.signaling. | 1.272 | 0.831 | 1.946 | 0.268015385 | 312 | 0.394140272 |
| pathway | | | | | | |
| X.ID.100114_1.NAME.role.of.mal.in.rho.mediated. | 1.264 | 0.816 | 1.956 | 0.293873448 | 312 | 0.423855935 |
| activation.of.srf | | | | | | |
| X.ID.200187_1.NAME.Aurora.A.signaling | 1.24 | 0.815 | 1.885 | 0.314611087 | 312 | 0.445204368 |
| X.ID.200164_1.NAME.Internalization.of.ErbB1 | 0.81 | 0.533 | 1.23 | 0.322973631 | 312 | 0.447041201 |
| X.ID.100194_1.NAME.ctcf..first.multivalent.nuclear. | 1.235 | 0.809 | 1.885 | 0.327830214 | 312 | 0.447041201 |
| factor | | | | | | |
| X.ID.500799_1.NAME.Hormone.sensitive.lipase.. | 1.233 | 0.806 | 1.888 | 0.333932038 | 312 | 0.447230408 |
| HSL..mediated.triacylglycerol.hydrolysis | | | | | | |
| X.ID.100047_1.NAME.ras.signaling.pathway | 0.816 | 0.537 | 1.24 | 0.341248184 | 312 | 0.449010768 |
| X.ID.200144_1.NAME.PDGFR.beta.signaling. | 0.824 | 0.544 | 1.25 | 0.363082087 | 312 | 0.469502699 |
| pathway | | | | | | |
| X.ID.200102_1.NAME.FoxO.family.signaling | 0.827 | 0.545 | 1.253 | 0.369512168 | 312 | 0.469718857 |
| X.ID.200070_3.NAME.LKB1.signaling.events | 0.836 | 0.55 | 1.271 | 0.402141827 | 312 | 0.49978264 |
| X.ID.100082_1.NAME.thrombin.signaling.and. | 1.193 | 0.786 | 1.811 | 0.40648988 | 312 | 0.49978264 |
| protease.activated.receptors | | | | | | |
| X.ID.100241_1.NAME.antisense.pathway | 1.186 | 0.784 | 1.794 | 0.418953699 | 312 | 0.506798829 |
| X.ID.200220_1.NAME.Notch.mediated.HES. | 1.186 | 0.779 | 1.805 | 0.426617516 | 312 | 0.507877995 |
| HEY.network | | | | | | |
| X.ID.100037_1.NAME.how.does.salmonella. | 1.174 | 0.767 | 1.796 | 0.460209036 | 312 | 0.539307464 |
| hijack.a.cell | | | | | | |
| X.ID.100252_1.NAME.agrin.in.postsynaptic.differentiation | 1.169 | 0.764 | 1.789 | 0.471225621 | 312 | 0.543721871 |
| X.ID.100211_1.NAME.role.of.pi3k.subunit.p85. | 0.884 | 0.584 | 1.338 | 0.559492581 | 312 | 0.635787024 |
| in.regulation.of.actin.organization.and.cell. | | | | | | |
| migration | | | | | | |
| X.ID.200145_5.NAME.Neurotrophic.factor.mediated. | 1.124 | 0.741 | 1.703 | 0.582511248 | 312 | 0.65206483 |
| Trk.receptor.signaling | | | | | | |
| X.ID.500592_1.NAME.Signaling.by.BMP | 1.117 | 0.737 | 1.693 | 0.6009142 | 312 | 0.662773015 |
| X.ID.200165_1.NAME.Hedgehog.signaling.events. | 1.109 | 0.731 | 1.682 | 0.626355912 | 312 | 0.680821644 |
| mediated.by.Gli.proteins | | | | | | |
| X.ID.200026_3.NAME.TCR.signaling.in.naive. | 1.097 | 0.726 | 1.66 | 0.659721614 | 312 | 0.706844586 |
| CD4..T.cells | | | | | | |
| X.ID.100244_3.NAME.alk.in.cardiac.myocytes | 1.076 | 0.707 | 1.637 | 0.73393791 | 312 | 0.775286525 |
| X.ID.200175_2.NAME.Signaling.events.mediated. | 1.063 | 0.701 | 1.612 | 0.773202664 | 312 | 0.805419441 |
| by.Stem.cell.factor.receptor..c.Kit. | | | | | | |
| X.ID.200006_1.NAME.Signaling.events.mediated. | 0.952 | 0.628 | 1.443 | 0.815010949 | 312 | 0.837340016 |
| by.PRL | | | | | | |
| X.ID.200022_1.NAME.Signaling.events.mediated. | 0.984 | 0.65 | 1.491 | 0.940165107 | 312 | 0.952870041 |
| by.HDAC.Class.II | | | | | | |
| X.ID.200114_2.NAME.Direct.p53.effectors | 0.989 | 0.653 | 1.499 | 0.959381886 | 312 | 0.959381886 |

TABLE 15
Colon cancer Model E. Hazard ratios (95% CI, p values, size of the validation
cohort and q values) of patients' MDS based classification. A univariate Cox proportional
hazards model was fit to each of the top ranked subnetwork markers (n_{Breast} = 50, n_{Colon} = 75,
n_{NSCLC} = 25 and n_{Ovarian} = 50) and subsequently applied to predict patient risk score in the
validation cohort. The survival differences between the predicted groups were assessed
using Kaplan-Meier analysis.

| | | 95% CI | 95% CI | | | |
|---|---|---|---|---|---|---|
| Subnetwork module | HR | lower | upper | P | n | Q |
| X.ID.100062_2.NAME.prion.pathway | 3.597 | 2.037 | 6.352 | 1.0301E−05 | 312 | 0.000772577 |
| X.ID.200017_1.NAME.p38.MAPK.signaling.pathway | 0.598 | 0.384 | 0.932 | 0.023104372 | 312 | 0.488710432 |
| X.ID.500866_1.NAME.mRNA.Splicing...Major.Pathway | 0.613 | 0.4 | 0.94 | 0.024812654 | 312 | 0.488710432 |
| X.ID.200066_2.NAME.CDC42.signaling.events | 0.618 | 0.404 | 0.944 | 0.026064556 | 312 | 0.488710432 |
| X.ID.200190_1.NAME.Class.I.PI3K.signaling.events. | 1.573 | 1.035 | 2.393 | 0.034101243 | 312 | 0.511518647 |
| medicated.by.Akt | | | | | | |
| X.ID.100174_2.NAME.er.associated.degradation..erad.. | 0.669 | 0.439 | 1.018 | 0.060803666 | 312 | 0.723862482 |
| pathway | | | | | | |
| X.ID.500655_1.NAME.Processing.of.Capped.Intron. | 0.689 | 0.453 | 1.048 | 0.081343565 | 312 | 0.723862482 |
| Containing.Pre.mRNA | | | | | | |
| X.ID.100029_1.NAME.sprouty.regulation.of.tyrosine. | 0.676 | 0.434 | 1.053 | 0.08347194 | 312 | 0.723862482 |
| kinase.signals | | | | | | |
| X.ID.200093_3.NAME.CXCR4.mediated.signaling. | 0.693 | 0.455 | 1.055 | 0.087372705 | 312 | 0.723862482 |
| events | | | | | | |
| X.ID.100083_1.NAME.p53.signaling.pathway | 0.712 | 0.466 | 1.088 | 0.116249508 | 312 | 0.723862482 |
| X.ID.200034_1.NAME.HIF.2.alpha.transcription.factor. | 1.392 | 0.92 | 2.106 | 0.117344662 | 312 | 0.723862482 |
| network | | | | | | |
| X.ID.500101_1.NAME.CHL1.interactions | 1.4 | 0.914 | 2.143 | 0.121995326 | 312 | 0.723862482 |
| X.ID.200102_1.NAME.FoxO.family.signaling | 1.382 | 0.913 | 2.093 | 0.126360312 | 312 | 0.723862482 |
| X.ID.100119_1.NAME.keratinocyte.differentiation | 1.397 | 0.901 | 2.166 | 0.135120997 | 312 | 0.723862482 |
| X.ID.500128_1.NAME.Insulin.Synthesis.and.Processing | 0.753 | 0.495 | 1.147 | 0.187007874 | 312 | 0.860760127 |
| X.ID.200070_3.NAME.LKB1.signaling.events | 1.324 | 0.867 | 2.022 | 0.193265873 | 312 | 0.860760127 |
| X.ID.100195_1.NAME.sumoylation.as.a.mechanism.to. | 0.756 | 0.496 | 1.154 | 0.195105629 | 312 | 0.860760127 |
| modulate.ctbp.dependent.gene.responses | | | | | | |
| X.ID.200040_1.NAME.Signaling.events.mediated.by. | 0.772 | 0.506 | 1.178 | 0.230516154 | 312 | 0.960483975 |
| PTP1B | | | | | | |
| X.ID.200173_1.NAME.Signaling.mediated.by.p38.alpha. | 0.78 | 0.512 | 1.19 | 0.249437929 | 312 | 0.984623405 |
| and.p38.beta | | | | | | |
| X.ID.200134_1.NAME.Urokinase.type.plasminogen. | 0.788 | 0.519 | 1.197 | 0.264662423 | 312 | 0.992484085 |
| activator..uPA..and.uPAR.mediated.signaling | | | | | | |
| X.ID.100145_1.NAME.hypoxia.inducible.factor.in.the. | 0.796 | 0.524 | 1.212 | 0.287890714 | 312 | 0.99315991 |
| cardivascular.system | | | | | | |
| X.ID.100095_2.NAME.ras.independent.pathway.in.nk. | 0.802 | 0.529 | 1.216 | 0.297992372 | 312 | 0.99315991 |
| cell.mediated.cytotoxicity. | | | | | | |
| X.ID.200050_1.NAME.EPHB.forward.signaling | 0.803 | 0.529 | 1.22 | 0.304572955 | 312 | 0.99315991 |
| X.ID.200189_1.NAME.Insulin.mediated.glucose. | 1.233 | 0.811 | 1.875 | 0.326981263 | 312 | 0.99315991 |
| transport | | | | | | |
| X.ID.500841_1.NAME.DARPP.32.events | 0.816 | 0.532 | 1.25 | 0.348992114 | 312 | 0.99315991 |
| X.ID.100116_3.NAME.lissencephaly.gene..lis1..in. | 1.222 | 0.801 | 1.864 | 0.352406742 | 312 | 0.99315991 |
| neuronal.migration.and.development | | | | | | |
| X.ID.500455_1.NAME.ERK.MAPK.targets | 0.827 | 0.546 | 1.252 | 0.369196143 | 312 | 0.99315991 |
| X.ID.200039_1.NAME.Signaling.events.mediated.by. | 0.832 | 0.549 | 1.26 | 0.384310554 | 312 | 0.99315991 |
| Hepatocyte.Growth.Factor.Receptor..c.Met. | | | | | | |
| X.ID.100144_1.NAME.hiv.1.nef..negative.effector.of.fas. | 1.197 | 0.792 | 1.81 | 0.393866294 | 312 | 0.99315991 |
| and.tnf | | | | | | |
| X.ID.200128_1.NAME.Syndecan.4.mediated.signaling. | 0.839 | 0.555 | 1.27 | 0.40710537 | 312 | 0.99315991 |
| events | | | | | | |
| X.ID.200012_3.NAME.LPA.receptor.mediated.events | 1.183 | 0.78 | 1.795 | 0.429853047 | 312 | 0.99315991 |
| X.ID.500652_1.NAME.Generic.Transcription.Pathway | 0.848 | 0.559 | 1.286 | 0.437284745 | 312 | 0.99315991 |
| X.ID.200004_3.NAME.Endothelins | 0.858 | 0.564 | 1.304 | 0.472066176 | 312 | 0.99315991 |
| X.ID.100059_2.NAME.phosphoinositides.and.their. | 0.859 | 0.564 | 1.306 | 0.476378762 | 312 | 0.99315991 |
| downstream.targets | | | | | | |
| X.ID.200183_2.NAME.a6b1.and.a6b4.Integrin.signaling | 0.866 | 0.57 | 1.314 | 0.497687825 | 312 | 0.99315991 |
| X.ID.100085_1.NAME.p38.mapk.signaling.pathway | 0.872 | 0.573 | 1.327 | 0.523048149 | 312 | 0.99315991 |
| X.ID.100137_1.NAME.skeletal.muscle.hypertrophy.is. | 1.143 | 0.75 | 1.743 | 0.534150884 | 312 | 0.99315991 |
| regulated.via.akt.mtor.pathway | | | | | | |
| X.ID.100197_1.NAME.regulation.of.spermatogenesis.by. | 1.135 | 0.75 | 1.716 | 0.549472284 | 312 | 0.99315991 |
| crem | | | | | | |
| X.ID.200129_1.NAME.ATF.2.transcription.factor. | 0.88 | 0.577 | 1.342 | 0.553288442 | 312 | 0.99315991 |
| network | | | | | | |
| X.ID.200064_1.NAME.Wnt.signaling.network | 1.128 | 0.743 | 1.712 | 0.571715233 | 312 | 0.99315991 |
| X.ID.200063_1.NAME.Regulation.of.p38.alpha.and.p38. | 0.896 | 0.587 | 1.368 | 0.611149846 | 312 | 0.99315991 |
| beta | | | | | | |
| X.ID.500522_1.NAME.Regulation.of.gene.expression.in. | 0.898 | 0.593 | 1.36 | 0.611725724 | 312 | 0.99315991 |
| beta.cells | | | | | | |
| X.ID.100152_1.NAME.inactivation.of.gsk3.by.akt. | 0.901 | 0.593 | 1.371 | 0.627424283 | 312 | 0.99315991 |
| causes.accumulation.of.b.catenin.in.alveolar.macrophages | | | | | | |
| X.ID.200175_6.NAME.Signaling.events.mediated.by. | 0.903 | 0.592 | 1.377 | 0.636527622 | 312 | 0.99315991 |
| Stem.cell.factor.receptor..c.Kit. | | | | | | |
| X.ID.100056_1.NAME.rac1.cell.motility.signaling. | 0.91 | 0.599 | 1.382 | 0.65828476 | 312 | 0.99315991 |
| pathway | | | | | | |
| X.ID.100008_1.NAME.ucalpain.and.friends.in.cell. | 0.914 | 0.592 | 1.409 | 0.682553606 | 312 | 0.99315991 |
| spread | | | | | | |
| X.ID.200175_2.NAME.Signaling.events.mediated.by. | 0.919 | 0.607 | 1.39 | 0.688216372 | 312 | 0.99315991 |
| Stem.cell.factor.receptor..c.Kit. | | | | | | |
| X.ID.100084_1.NAME.hypoxia.and.p53.in.the. | 0.919 | 0.606 | 1.394 | 0.691473601 | 312 | 0.99315991 |
| cardiovascular.system | | | | | | |
| X.ID.500068_1.NAME.Fanconi.Anemia.pathway | 0.92 | 0.599 | 1.414 | 0.70354192 | 312 | 0.99315991 |
| X.ID.200011_1.NAME.Aurora.B.signaling | 0.923 | 0.608 | 1.399 | 0.70496446 | 312 | 0.99315991 |
| X.ID.200198_1.NAME.BARD1.signaling.events | 0.93 | 0.611 | 1.416 | 0.735628793 | 312 | 0.99315991 |
| X.ID.100113_1.NAME.mapkinase.signaling.pathway | 0.935 | 0.616 | 1.419 | 0.752200886 | 312 | 0.99315991 |
| X.ID.200003_1.NAME.Fc.epsilon.receptor.I.signaling.in. | 0.937 | 0.619 | 1.416 | 0.755956158 | 312 | 0.99315991 |
| mast.cells | | | | | | |
| X.ID.200006_1.NAME.Signaling.events.mediated.by. | 1.068 | 0.704 | 1.622 | 0.756076433 | 312 | 0.99315991 |
| PRL | | | | | | |
| X.ID.200201_1.NAME.Endogenous.TLR.signaling | 1.063 | 0.697 | 1.621 | 0.776143398 | 312 | 0.99315991 |
| X.ID.100047_2.NAME.ras.signaling.pathway | 0.944 | 0.614 | 1.451 | 0.792352627 | 312 | 0.99315991 |
| X.ID.200085_1.NAME.Role.of.Calcineurin.dependent. | 0.944 | 0.605 | 1.472 | 0.798855981 | 312 | 0.99315991 |
| NFAT.signaling.in.lymphocytes | | | | | | |
| X.ID.100111_1.NAME.mcalpain.and.friends.in.cell. | 0.949 | 0.628 | 1.436 | 0.80568886 | 312 | 0.99315991 |
| motility | | | | | | |
| X.ID.500575_2.NAME.RNA.Polymerase.I.Transcription. | 0.949 | 0.626 | 1.44 | 0.807078666 | 312 | 0.99315991 |
| Initiation | | | | | | |
| X.ID.200166_2.NAME.Caspase.cascade.in.apoptosis | 1.05 | 0.691 | 1.596 | 0.818765372 | 312 | 0.99315991 |
| X.ID.100026_2.NAME.tntf.stress.related.signaling | 0.956 | 0.631 | 1.45 | 0.833110681 | 312 | 0.99315991 |
| X.ID.100132_1.NAME.signal.transduction.through.il1r | 0.958 | 0.631 | 1.454 | 0.841634897 | 312 | 0.99315991 |
| X.ID.200139_1.NAME.BMP.receptor.signaling | 0.97 | 0.641 | 1.466 | 0.883307422 | 312 | 0.99315991 |
| X.ID.200024_1.NAME.Signaling.events.mediated.by. | 1.027 | 0.67 | 1.574 | 0.902108286 | 312 | 0.99315991 |
| HDAC.Class.III | | | | | | |
| X.ID.100105_1.NAME.signal.dependent.regulation.of. | 1.025 | 0.675 | 1.557 | 0.907600353 | 312 | 0.99315991 |
| myogenesis.by.corepressor.mitr | | | | | | |
| X.ID.200008_1.NAME.RhoA.signaling.pathway | 0.975 | 0.629 | 1.51 | 0.908814912 | 312 | 0.99315991 |
| X.ID.100098_1.NAME.nfat.and.hypertrophy.of.the.heart. | 0.98 | 0.64 | 1.499 | 0.924898188 | 312 | 0.99315991 |
| X.ID.100041_1.NAME.rho.cell.motility.signaling. | 0.982 | 0.649 | 1.485 | 0.931839757 | 312 | 0.99315991 |
| pathway | | | | | | |
| X.ID.100148_1.NAME.control.of.skeletal.myogenesis. | 1.015 | 0.671 | 1.536 | 0.943976749 | 312 | 0.99315991 |
| by.hdac.and.calcium.calmodulin.dependent.kinase..camk. | | | | | | |
| X.ID.100233_1.NAME.regulation.of.bad.phosphorylation | 1.01 | 0.666 | 1.532 | 0.963254069 | 312 | 0.99315991 |
| X.ID.200062_1.NAME.Nectin.adhesion.pathway | 0.991 | 0.649 | 1.515 | 0.967731893 | 312 | 0.99315991 |
| X.ID.500120_1.NAME.Adherens.junctions.interactions | 0.995 | 0.656 | 1.508 | 0.979952522 | 312 | 0.99315991 |
| X.ID.200187_1.NAME.Aurora.A.signaling | 1.003 | 0.661 | 1.52 | 0.990371699 | 312 | 0.99315991 |
| X.ID.200079_1.NAME.Signaling.events.mediated.by. | 1.003 | 0.661 | 1.52 | 0.990515791 | 312 | 0.99315991 |
| HDAC.Class.I | | | | | | |
| X.ID.100032_1.NAME.map.kinase.inactivation.of.smrt. | 1.002 | 0.662 | 1.516 | 0.99315991 | 312 | 0.99315991 |
| corepressor | | | | | | |

TABLE 16
NSCLC cancer Model N + E. Hazard ratios (95% CI, p values, size of the
validation cohort and q values) of patients' MDS based classification. A univariate Cox
proportional hazards model was fit to each of the top ranked subnetwork markers (n_{Breast} = 50,
n_{Colon} = 75, n_{NSCLC} = 25 and n_{Ovarian} = 50) and subsequently applied to predict patient risk score in
the validation cohort. The survival differences between the predicted groups were assessed
using Kaplan-Meier analysis.

| | | 95% CI | 95% CI | | | |
|---|---|---|---|---|---|---|
| Subnetwork module | HR | lower | upper | P | n | Q |
| X.ID.100221_2.NAME.role.of.egf.receptor. | 1.622 | 1.165 | 2.259 | 0.004187789 | 369 | 0.08648986 |
| transactivation.by.gpcrs.in.cardiac.hypertrophy | | | | | | |
| X.ID.200211_1.NAME.Alpha.synuclein.signaling | 1.542 | 1.119 | 2.126 | 0.008201517 | 369 | 0.08648986 |
| X.ID.200126_2.NAME.ErbB1.downstream. | 1.514 | 1.098 | 2.087 | 0.011301659 | 369 | 0.08648986 |
| signaling | | | | | | |
| X.ID.200079_1.NAME.Signaling.events.mediated. | 1.502 | 1.086 | 2.076 | 0.013838377 | 369 | 0.08648986 |
| by.HDAC.Class.I | | | | | | |
| X.ID.100170_2.NAME.erk1.erk2.mapk.signaling. | 1.431 | 1.03 | 1.988 | 0.032610164 | 369 | 0.14938698 |
| pathway | | | | | | |
| X.ID.200064_1.NAME.Wnt.signaling.network | 1.401 | 1.015 | 1.936 | 0.040599267 | 369 | 0.14938698 |
| X.ID.100056_1.NAME.rac1.cell.motility.signaling. | 1.401 | 1.009 | 1.944 | 0.043810897 | 369 | 0.14938698 |
| pathway | | | | | | |
| X.ID.200102_1.NAME.FoxO.family.signaling | 1.382 | 1.003 | 1.905 | 0.047803834 | 369 | 0.14938698 |
| X.ID.200173_1.NAME.Signaling.mediated.by.p38. | 1.374 | 0.995 | 1.897 | 0.053872131 | 369 | 0.14964481 |
| alpha.and.p38.beta | | | | | | |
| X.ID.200061_2.NAME.Presenilin.action.in.Notch. | 1.346 | 0.976 | 1.857 | 0.07025369 | 369 | 0.17563422 |
| and.Wnt.signaling | | | | | | |
| X.ID.100113_1.NAME.mapkinase.signaling. | 1.301 | 0.942 | 1.798 | 0.110116286 | 369 | 0.25026429 |
| pathway | | | | | | |
| X.ID.100085_1.NAME.p38.mapk.signaling. | 1.264 | 0.914 | 1.748 | 0.156215167 | 369 | 0.32544826 |
| pathway | | | | | | |
| X.ID.100185_1.NAME.regulation.of.map.kinase. | 1.235 | 0.894 | 1.708 | 0.200617013 | 369 | 0.38580195 |
| pathways.through.dual.specificity.phosphatases | | | | | | |
| X.ID.100159_1.NAME.cell.cycle..g2.m.checkpoint | 1.209 | 0.876 | 1.669 | 0.248082058 | 369 | 0.4278173 |
| X.ID.500655_1.NAME.Processing.of.Capped. | 1.204 | 0.874 | 1.66 | 0.256690382 | 369 | 0.4278173 |
| Intron.Containing.Pre.mRNA | | | | | | |
| X.ID.200128_1.NAME.Syndecan.4.mediated. | 1.163 | 0.844 | 1.604 | 0.355362643 | 369 | 0.55525413 |
| signaling.events | | | | | | |
| X.ID.200215_2.NAME.Regulation.of. | 0.875 | 0.635 | 1.206 | 0.415517134 | 369 | 0.61105461 |
| retinoblastoma.protein | | | | | | |
| X.ID.100046_1.NAME.rb.tumor.suppressor. | 1.134 | 0.823 | 1.563 | 0.441013116 | 369 | 0.61251822 |
| checkpoint.signaling.in.response.to.dna.damage | | | | | | |
| X.ID.500866_1.NAME.mRNA.Splicing...Major. | 0.909 | 0.659 | 1.252 | 0.558288245 | 369 | 0.7345898 |
| Pathway | | | | | | |
| X.ID.200185_1.NAME.Syndecan.2.mediated. | 0.926 | 0.672 | 1.275 | 0.636241889 | 369 | 0.79530236 |
| signaling.events | | | | | | |
| X.ID.500652_1.NAME.Generic.Transcription. | 0.946 | 0.686 | 1.305 | 0.734515478 | 369 | 0.84285684 |
| Pathway | | | | | | |
| X.ID.200053_1.NAME.Validated.transcriptional. | 1.056 | 0.765 | 1.457 | 0.741714021 | 369 | 0.84285684 |
| targets.of.AP1.family.members.Fra1.and.Fra2 | | | | | | |
| X.ID.200063_1.NAME.Regulation.of.p38.alpha. | 0.959 | 0.696 | 1.321 | 0.796976068 | 369 | 0.85548221 |
| and.p38.beta | | | | | | |
| X.ID.100119_1.NAME.keratinocyte.differentiation | 1.038 | 0.753 | 1.431 | 0.821262922 | 369 | 0.85548221 |
| X.ID.100123_1.NAME.integrin.signaling.pathway | 0.986 | 0.715 | 1.36 | 0.930533476 | 369 | 0.93053348 |

TABLE 16
NSCLC cancer Model N. Hazard ratios (95% CI, p values, size of the validation
cohort and q values) of patients' MDS based classification. A univariate Cox proportional
hazards model was fit to each of the top ranked subnetwork markers (n_{Breast} = 50, n_{Colon} = 75,
n_{NSCLC} = 25 and n_{Ovarian} = 50) and subsequently applied to predict patient risk score in the
validation cohort. The survival differences between the predicted groups were assessed
using Kaplan-Meier analysis.

| | | 95% CI | 95% CI | | | |
|---|---|---|---|---|---|---|
| Subnetwork module | HR | lower | upper | P | n | Q |
| X.ID.200206_1.NAME.Trk.receptor. | 1.745 | 1.259 | 2.419 | 0.000821978 | 369 | 0.02054945 |
| signaling.mediated.by.the.MAPK.pathway | | | | | | |
| X.ID.200180_1.NAME.Effects.of. | 1.668 | 1.206 | 2.307 | 0.001968758 | 369 | 0.02356251 |
| Botulinum.toxin | | | | | | |
| X.ID.200011_1.NAME.Aurora.B.signaling | 1.635 | 1.184 | 2.258 | 0.002827501 | 369 | 0.02356251 |
| X.ID.500150_1.NAME.Glutamate. | 1.599 | 1.158 | 2.208 | 0.004391549 | 369 | 0.02461353 |
| Neurotransmitter.Release.Cycle | | | | | | |
| X.ID.100221_2.NAME.role.of.egf.receptor. | 1.595 | 1.152 | 2.208 | 0.004922707 | 369 | 0.02461353 |
| transactivation.by.gpcrs.in.cardiac. | | | | | | |
| hypertrophy | | | | | | |
| X.ID.100018_2.NAME.trefoil.factors. | 1.538 | 1.111 | 2.13 | 0.009476892 | 369 | 0.03948705 |
| initiate.mucosal.healing | | | | | | |
| X.ID.100059_2.NAME.phosphoinositides. | 1.492 | 1.081 | 2.058 | 0.014942639 | 369 | 0.05336657 |
| and.their.downstream.targets | | | | | | |
| X.ID.200064_1.NAME.Wnt.signaling. | 1.465 | 1.058 | 2.027 | 0.021400335 | 369 | 0.06687605 |
| network | | | | | | |
| X.ID.100056_1.NAME.rac1.cell.motility. | 1.394 | 1.008 | 1.929 | 0.044716956 | 369 | 0.12159078 |
| signaling.pathway | | | | | | |
| X.ID.200122_1.NAME.Integrins.in. | 1.38 | 1.002 | 1.902 | 0.04863631 | 369 | 0.12159078 |
| angiogenesis | | | | | | |
| X.ID.100113_1.NAME.mapkinase.signaling. | 1.363 | 0.99 | 1.879 | 0.058003154 | 369 | 0.12224538 |
| pathway | | | | | | |
| X.ID.100085_1.NAME.p38.mapk.signaling. | 1.368 | 0.989 | 1.894 | 0.058677782 | 369 | 0.12224538 |
| pathway | | | | | | |
| X.ID.100046_1.NAME.rb.tumor.suppressor. | 1.321 | 0.953 | 1.83 | 0.09469857 | 369 | 0.1771489 |
| checkpoint.signaling.in.response.to.dna. | | | | | | |
| damage | | | | | | |
| X.ID.200211_1.NAME.Alpha.synuclein. | 1.31 | 0.95 | 1.805 | 0.099203382 | 369 | 0.1771489 |
| signaling | | | | | | |
| X.ID.200173_1.NAME.Signaling.mediated. | 1.273 | 0.923 | 1.757 | 0.141417864 | 369 | 0.23569644 |
| by.p38.alpha.and.p38.beta | | | | | | |
| X.ID.200165_1.NAME.Hedgehog.signaling. | 1.262 | 0.916 | 1.738 | 0.155425828 | 369 | 0.24285286 |
| events.mediated.by.Gli.proteins | | | | | | |
| X.ID.200199_1.NAME.p53.pathway | 1.231 | 0.892 | 1.698 | 0.20684633 | 369 | 0.30418578 |
| X.ID.100159_1.NAME.cell.cycle..g2.m. | 1.214 | 0.88 | 1.675 | 0.238359302 | 369 | 0.33105459 |
| checkpoint | | | | | | |
| X.ID.200185_1.NAME.Syndecan.2. | 0.853 | 0.618 | 1.177 | 0.332765386 | 369 | 0.43784919 |
| mediated.signaling.events | | | | | | |
| X.ID.200128_1.NAME.Syndecan.4. | 1.153 | 0.837 | 1.59 | 0.382809955 | 369 | 0.47851244 |
| mediated.signaling.events | | | | | | |
| X.ID.200102_1.NAME.FoxO.family. | 1.129 | 0.819 | 1.557 | 0.457007366 | 369 | 0.53135022 |
| signaling | | | | | | |
| XID.100053_1.NAME.sumoylation.by. | 1.125 | 0.815 | 1.552 | 0.4740281 | 369 | 0.53135022 |
| ranbp2.regulates.transcriptional.repression | | | | | | |
| X.ID.200145_2.NAME.Neurotrophic. | 1.12 | 0.812 | 1.544 | 0.4888422 | 369 | 0.53135022 |
| factor.mediated.Trk.receptor.signaling | | | | | | |
| X.ID.200215_2.NAME.Regulation.of. | 1.033 | 0.749 | 1.423 | 0.844664419 | 369 | 0.8688818 |
| retinoblastoma.protein | | | | | | |
| X.ID.500087_1.NAME.NCAM1.interactions | 0.973 | 0.707 | 1.341 | 0.868881801 | 369 | 0.8688818 |

TABLE 16
NSCLC cancer Model E. Hazard ratios (95% CI, p values, size of the validation
cohort and q values) of patients' MDS based classification. A univariate Cox proportional
hazards model was fit to each of the top ranked subnetwork markers (n_{Breast} = 50, n_{Colon} = 75,
n_{NSCLC} = 25 and n_{Ovarian} = 50) and subsequently applied to predict patient risk score in the
validation cohort. The survival differences between the predicted groups were assessed
using Kaplan-Meier analysis.

| | | 95% CI | 95% CI | | | |
|---|---|---|---|---|---|---|
| Subnetwork module | HR | lower | upper | P | n | Q |
| X.ID.200063_1.NAME.Regulation.of.p38.alpha. | 0.675 | 0.489 | 0.931 | 0.01673499 | 369 | 0.4183748 |
| and.p38.beta | | | | | | |
| X.ID.200079_1.NAME.Signaling.events.mediated. | 1.346 | 0.977 | 1.855 | 0.069241709 | 369 | 0.496036 |
| by.HDAC.Class.I | | | | | | |
| X.ID.200211_1.NAME.Alpha.synuclein.signaling | 1.339 | 0.971 | 1.846 | 0.075214647 | 369 | 0.496036 |
| X.ID.100113_1.NAME.mapkinase.signaling. | 1.343 | 0.966 | 1.869 | 0.079365754 | 369 | 0.496036 |
| pathway | | | | | | |
| X.ID.200173_1.NAME.Signaling.mediated.by.p38. | 1.272 | 0.922 | 1.755 | 0.142998926 | 369 | 0.5848696 |
| alpha.and.p38.beta | | | | | | |
| X.ID.500655_1.NAME.Processing.of.Capped. | 1.253 | 0.91 | 1.726 | 0.167509794 | 369 | 0.5848696 |
| Intron.Containing.Pre.mRNA | | | | | | |
| X.ID.100072_1.NAME.platelet.amyloid.precursor. | 1.247 | 0.905 | 1.717 | 0.177647326 | 369 | 0.5848696 |
| protein.pathway | | | | | | |
| X.ID.200024_1.NAME.Signaling.events.mediated. | 1.238 | 0.898 | 1.706 | 0.193439799 | 369 | 0.5848696 |
| by.HDAC.Class.III | | | | | | |
| X.ID.200022_1.NAME.Signaling.events.mediated. | 0.813 | 0.587 | 1.125 | 0.210553051 | 369 | 0.5848696 |
| by.HDAC.Class.II | | | | | | |
| X.ID.100170_2.NAME.erk1.erk2.mapk.signaling. | 1.148 | 0.833 | 1.584 | 0.398611157 | 369 | 0.9568862 |
| pathway | | | | | | |
| X.ID.200126_2.NAME.ErbB1.downstream. | 1.134 | 0.823 | 1.562 | 0.442627068 | 369 | 0.9568862 |
| signaling | | | | | | |
| X.ID.200053_1.NAME.Validated.transcriptional. | 0.89 | 0.645 | 1.229 | 0.478276007 | 369 | 0.9568862 |
| targets.of.AP1.family.members.Fra1.and.Fra2 | | | | | | |
| X.ID.100185_1.NAME.regulation.of.map.kinase. | 0.895 | 0.65 | 1.233 | 0.497580833 | 369 | 0.9568862 |
| pathways.through.dual.specificity.phosphatases | | | | | | |
| X.ID.100123_1.NAME.integrin.signaling.pathway | 0.915 | 0.662 | 1.266 | 0.592333092 | 369 | 0.9814177 |
| X.ID.500406_1.NAME.Chemokine.receptors.bind. | 0.923 | 0.667 | 1.277 | 0.629311548 | 369 | 0.9814177 |
| chemokines | | | | | | |
| X.ID.500652_1.NAME.Generic.Transcription. | 0.935 | 0.678 | 1.288 | 0.679694026 | 369 | 0.9814177 |
| Pathway | | | | | | |
| X.ID.100164_1.NAME.fibrinolysis.pathway | 0.938 | 0.678 | 1.296 | 0.696817772 | 369 | 0.9814177 |
| X.ID.100091_1.NAME.proteolysis.and.signaling. | 1.062 | 0.771 | 1.464 | 0.712878499 | 369 | 0.9814177 |
| pathway.of.notch | | | | | | |
| X.ID.200102_1.NAME.FoxO.family.signaling | 1.045 | 0.758 | 1.439 | 0.789517563 | 369 | 0.9814177 |
| X.ID.200136_1.NAME.FOXM1.transcription. | 1.043 | 0.756 | 1.438 | 0.799535691 | 369 | 0.9814177 |
| factor.network | | | | | | |
| X.ID.200158_1.NAME.Retinoic.acid.receptors. | 1.027 | 0.745 | 1.417 | 0.869819964 | 369 | 0.9814177 |
| mediated.signaling | | | | | | |
| X.ID.100119_1.NAME.keratinocyte.differentiation | 1.021 | 0.741 | 1.407 | 0.900539691 | 369 | 0.9814177 |
| X.ID.100159_1.NAME.cell.cycle..g2.m.checkpoint | 0.98 | 0.709 | 1.354 | 0.902904319 | 369 | 0.9814177 |
| X.ID.500866_1.NAME.mRNA.Splicing...Major. | 0.991 | 0.719 | 1.366 | 0.955978645 | 369 | 0.9896447 |
| Pathway | | | | | | |
| X.ID.200061_2.NAME.Presenilin.action.in.Notch. | 1.002 | 0.725 | 1.384 | 0.989644744 | 369 | 0.9896447 |
| and.Wnt.signaling | | | | | | |

TABLE 17
Ovarian cancer Model N + E. Hazard ratios (95% CI, p values, size of the
validation cohort and q values) of patients' MDS based classification. A univariate Cox
proportional hazards model was fit to each of the top ranked subnetwork markers (n_{Breast} = 50,
n_{Colon} = 75, n_{NSCLC} = 25 and n_{Ovarian} = 50) and subsequently applied to predict patient risk score in
the validation cohort. The survival differences between the predicted groups were assessed
using Kaplan-Meier analysis.

| | | 95% CI | 95% CI | | | |
|---|---|---|---|---|---|---|
| Subnetwork module | HR | lower | upper | P | n | Q |
| X.ID.200064_1.NAME.Wnt.signaling.network | 1.444 | 1.192 | 1.749 | 0.000174493 | 865 | 0.00872465 |
| X.ID.200190_1.NAME.Class.I.PI3K.signaling.events. | 1.349 | 1.114 | 1.634 | 0.002169951 | 865 | 0.05424877 |
| mediated.by.Akt | | | | | | |
| X.ID.200012_2.NAME.LPA.receptor.mediated.events | 1.32 | 1.088 | 1.602 | 0.004901338 | 865 | 0.08168897 |
| X.ID.200043_1.NAME.IL12.mediated.signaling. | 1.289 | 1.064 | 1.562 | 0.009599991 | 865 | 0.09109546 |
| events | | | | | | |
| X.ID.200199_1.NAME.p53.pathway | 1.285 | 1.06 | 1.557 | 0.010538369 | 865 | 0.09109546 |
| X.ID.100123_1.NAME.integrin.signaling.pathway | 1.277 | 1.054 | 1.548 | 0.012440149 | 865 | 0.09109546 |
| X.ID.200102_1.NAME.FoxO.family.signaling | 1.272 | 1.05 | 1.541 | 0.014116234 | 865 | 0.09109546 |
| X.ID.200040_1.NAME.Signaling.events.mediated.by. | 1.27 | 1.048 | 1.539 | 0.014575273 | 865 | 0.09109546 |
| PTP1B | | | | | | |
| X.ID.200153_1.NAME.ErbB.receptor.signaling. | 1.247 | 1.029 | 1.51 | 0.024061106 | 865 | 0.13367281 |
| network | | | | | | |
| X.ID.100113_1.NAME.mapkinase.signaling.pathway | 1.234 | 1.017 | 1.498 | 0.033434886 | 865 | 0.16717443 |
| X.ID.200185_1.NAME.Syndecan.2.mediated. | 1.207 | 0.995 | 1.464 | 0.056549884 | 865 | 0.2549652 |
| signaling.events | | | | | | |
| X.ID.200079_1.NAME.Signaling.events.mediated.by. | 1.201 | 0.991 | 1.455 | 0.061191647 | 865 | 0.2549652 |
| HDAC.Class.I | | | | | | |
| X.ID.500097_1.NAME.L1CAM.interactions | 1.179 | 0.973 | 1.428 | 0.092245374 | 865 | 0.28391935 |
| X.ID.200211_1.NAME.Alpha.synuclein.signaling | 1.179 | 0.973 | 1.428 | 0.092276202 | 865 | 0.28391935 |
| X.ID.100056_1.NAME.rac1.cell.motility.signaling. | 1.178 | 0.973 | 1.427 | 0.093248091 | 865 | 0.28391935 |
| pathway | | | | | | |
| X.ID.500866_1.NAME.mRNA.Splicing...Major. | 1.181 | 0.973 | 1.433 | 0.093296455 | 865 | 0.28391935 |
| Pathway | | | | | | |
| X.ID.200144_1.NAME.PDGFR.beta.signaling. | 1.178 | 0.971 | 1.43 | 0.096532578 | 865 | 0.28391935 |
| pathway | | | | | | |
| X.ID.100144_1.NAME.hiv.1.nef..negative.effector.of. | 1.169 | 0.963 | 1.418 | 0.113983692 | 865 | 0.29007849 |
| fas.and.tnf | | | | | | |
| X.ID.100008_1.NAME.ucalpain.and.friends.in.cell. | 1.166 | 0.963 | 1.413 | 0.11576819 | 865 | 0.29007849 |
| spread | | | | | | |
| X.ID.100178_1.NAME.regulation.of.eif.4e.and.p70s6. | 1.166 | 0.963 | 1.412 | 0.116031397 | 865 | 0.29007849 |
| kinase | | | | | | |
| X.ID.100169_1.NAME.mets.affect.on.macrophage. | 1.161 | 0.958 | 1.408 | 0.127658382 | 865 | 0.30202494 |
| differentiation | | | | | | |
| X.ID.200048_1.NAME.Calcineurin.regulated.NFAT. | 1.158 | 0.956 | 1.402 | 0.132890974 | 865 | 0.30202494 |
| dependent.transcription.in.lymphocytes | | | | | | |
| X.ID.100040_1.NAME.double.stranded.rna.induced. | 1.146 | 0.946 | 1.387 | 0.16280524 | 865 | 0.35392443 |
| gene.expression | | | | | | |
| X.ID.500945_1.NAME.Removal.of.DNA.patch. | 1.142 | 0.942 | 1.384 | 0.177241168 | 865 | 0.36925243 |
| containing.abasic.residue | | | | | | |
| X.ID.500655_1.NAME.Processing.of.Capped.Intron. | 0.881 | 0.727 | 1.068 | 0.19629573 | 865 | 0.39259146 |
| Containing.Pre.mRNA | | | | | | |
| X.ID.100168_1.NAME.extrinsic.prothrombin. | 1.126 | 0.929 | 1.364 | 0.22749333 | 865 | 0.4307507 |
| activation.pathway | | | | | | |
| X.ID.200183_2.NAME.a6b1.and.a6b4.Integrin. | 1.125 | 0.927 | 1.364 | 0.232605377 | 865 | 0.4307507 |
| signaling | | | | | | |
| X.ID.200165_1.NAME.Hedgehog.signaling.events. | 1.113 | 0.919 | 1.348 | 0.27404985 | 865 | 0.4892428 |
| mediated.by.Gli.proteins | | | | | | |
| X.ID.200085_1.NAME.Role.of.Calcineurin. | 1.11 | 0.915 | 1.346 | 0.290114058 | 865 | 0.4892428 |
| dependent.NFAT.signaling.in.lymphocytes | | | | | | |
| X.ID.200011_1.NAME.Aurora.B.signaling | 1.108 | 0.915 | 1.342 | 0.293545678 | 865 | 0.4892428 |
| X.ID.200148_1.NAME.C.MYB.transcription.factor. | 1.103 | 0.911 | 1.336 | 0.315551875 | 865 | 0.50895464 |
| network | | | | | | |
| X.ID.200126_2.NAME.ErbB1.downstream.signaling | 1.097 | 0.906 | 1.329 | 0.343099605 | 865 | 0.53609313 |
| X.ID.100022_1.NAME.t.cell.receptor.signaling. | 1.089 | 0.898 | 1.321 | 0.385035586 | 865 | 0.57340721 |
| pathway | | | | | | |
| X.ID.100041_1.NAME.rho.cell.motility.signaling. | 1.09 | 0.896 | 1.325 | 0.389916902 | 865 | 0.57340721 |
| pathway | | | | | | |
| X.ID.200022_1.NAME.Signaling.events.mediated.by. | 0.933 | 0.77 | 1.131 | 0.481338803 | 865 | 0.67779612 |
| HDAC.Class.II | | | | | | |
| X.ID.500652_1.NAME.Generic.Transcription.Pathway | 0.938 | 0.773 | 1.139 | 0.517815469 | 865 | 0.67779612 |
| X.ID.200128_1.NAME.Syndecan.4.mediated. | 1.065 | 0.879 | 1.29 | 0.518959389 | 865 | 0.67779612 |
| signaling.events | | | | | | |
| X.ID.200220_1.NAME.Notch.mediated.HES.HEY. | 1.065 | 0.878 | 1.292 | 0.522573259 | 865 | 0.67779612 |
| network | | | | | | |
| X.ID.200208_2.NAME.Downstream.signaling.in. | 1.063 | 0.875 | 1.292 | 0.539729353 | 865 | 0.67779612 |
| naive.CD8..T.cells | | | | | | |
| X.ID.200081_2.NAME.Regulation.of.Telomerase | 1.061 | 0.876 | 1.286 | 0.5422369 | 865 | 0.67779612 |
| X.ID.200187_1.NAME.Aurora.A.signaling | 1.059 | 0.875 | 1.282 | 0.557513304 | 865 | 0.67989427 |
| X.ID.200031_2.NAME.E2F.transcription.factor. | 0.953 | 0.787 | 1.154 | 0.623254093 | 865 | 0.74196916 |
| network | | | | | | |
| X.ID.200166_2.NAME.Caspase.cascade.in.apoptosis | 0.955 | 0.789 | 1.157 | 0.639905405 | 865 | 0.74407605 |
| X.ID.100221_2.NAME.role.of.egf.receptor. | 0.964 | 0.796 | 1.168 | 0.70834984 | 865 | 0.804943 |
| transactivation.by.gpcrs.in.cardiac.hypertrophy | | | | | | |
| X.ID.100183_1.NAME.phospholipids.as.signalling. | 1.027 | 0.847 | 1.244 | 0.787589453 | 865 | 0.86925308 |
| intermediaries | | | | | | |
| X.ID.500307_1.NAME.PECAM1.interactions | 0.976 | 0.806 | 1.183 | 0.806057069 | 865 | 0.86925308 |
| X.ID.100185_1.NAME.regulation.of.map.kinase. | 0.978 | 0.807 | 1.184 | 0.817097891 | 865 | 0.86925308 |
| pathways.through.dual.specificity.phosphatases | | | | | | |
| X.ID.100100_1.NAME.pkc.catalyzed.phosphorylation. | 0.983 | 0.811 | 1.192 | 0.863592704 | 865 | 0.89957573 |
| of.inhibitory.phosphoprotein.of.myosin.phosphatase | | | | | | |
| X.ID.100152_1.NAME.inactivation.of.gsk3.by.akt. | 1.009 | 0.833 | 1.222 | 0.929408409 | 865 | 0.94837593 |
| causes.accumulation.of.b.catenin.in.alveolar. | | | | | | |
| macrophages | | | | | | |
| X.ID.200024_1.NAME.Signaling.events.mediated.by. | 1.006 | 0.831 | 1.218 | 0.950671339 | 865 | 0.95067134 |
| HDAC.CIass.III | | | | | | |

TABLE 17
Ovarian cancer Model N. Hazard ratios (95% CI, p values, size of the validation
cohort and q values) of patients' MDS based classification. A univariate Cox proportional
hazards model was fit to each of the top ranked subnetwork markers (n_{Breast} = 50, n_{Colon} = 75,
n_{NSCLC} = 25 and n_{Ovarian} = 50) and subsequently applied to predict patient risk score in the
validation cohort. The survival differences between the predicted groups were assessed
using Kaplan-Meier analysis.

| | | 95% CI | 95% CI | | | |
|---|---|---|---|---|---|---|
| Subnetwork module | HR | lower | upper | P | n | Q |
| X.ID.100218_1.NAME.caspase.cascade.in. | 1.336 | 1.103 | 1.619 | 0.00306552 | 865 | 0.09559887 |
| apoptosis | | | | | | |
| X.ID.500799_1.NAME.Hormone.sensitive.lipase.. | 1.332 | 1.094 | 1.623 | 0.004366746 | 865 | 0.09559887 |
| HSL...mediated.triacylglycerol.hydrolysis | | | | | | |
| X.ID.200040_1.NAME.Signaling.events. | 1.307 | 1.079 | 1.584 | 0.006229085 | 865 | 0.09559887 |
| mediated.by.PTP1B | | | | | | |
| X.ID.200148_1.NAME.C.MYB.transcription. | 1.292 | 1.066 | 1.565 | 0.008901658 | 865 | 0.09559887 |
| factor.network | | | | | | |
| X.ID.200199_1.NAME.p53.pathway | 1.289 | 1.064 | 1.561 | 0.009559887 | 865 | 0.09559887 |
| X.ID.100008_1.NAME.ucalpain.and.friends.in. | 1.279 | 1.056 | 1.549 | 0.011962246 | 865 | 0.09968538 |
| cell.spread | | | | | | |
| X.ID.100204_2.NAME.apoptotic.signaling.in. | 1.265 | 1.044 | 1.532 | 0.016181432 | 865 | 0.11099122 |
| response.to.dna.damage | | | | | | |
| X.ID.100144_1.NAME.hiv.1.net.negative. | 1.261 | 1.041 | 1.527 | 0.017758595 | 865 | 0.11099122 |
| effector.of.fas.and.tnf | | | | | | |
| X.ID.500522_1.NAME.Regulation.of.gene. | 1.25 | 1.03 | 1.517 | 0.024174465 | 865 | 0.12193503 |
| expression.in.beta.cells | | | | | | |
| X.ID.200153_1.NAME.ErbB.receptor.signaling. | 1.246 | 1.028 | 1.509 | 0.024854062 | 865 | 0.12193503 |
| network | | | | | | |
| X.ID.200061_1.NAME.Presenilin.action.in. | 1.242 | 1.025 | 1.504 | 0.026825706 | 865 | 0.12193503 |
| Notch.and.Wnt.signaling | | | | | | |
| X.ID.200220_1.NAME.Notch.mediated.HES. | 1.217 | 1.004 | 1.475 | 0.045301395 | 865 | 0.17939405 |
| HEY.network | | | | | | |
| X.ID.200077_1.NAME.Circadian.rhythm. | 1.214 | 1.003 | 1.47 | 0.046776465 | 865 | 0.17939405 |
| pathway | | | | | | |
| X.ID.200138_1.NAME.Hypoxic.and.oxygen. | 1.211 | 1 | 1.468 | 0.050230334 | 865 | 0.17939405 |
| homeostasis.regulation.of.HIF.1.alpha | | | | | | |
| X.ID.200064_1.NAME.Wnt.signaling.network | 1.207 | 0.996 | 1.462 | 0.05456414 | 865 | 0.18188047 |
| X.ID.200012_2.NAME.LPA.receptor.mediated. | 1.205 | 0.993 | 1.461 | 0.058703019 | 865 | 0.18344693 |
| events | | | | | | |
| X.ID.200079_1.NAME.Signaling.events. | 1.192 | 0.984 | 1.445 | 0.073303665 | 865 | 0.20925644 |
| mediated.by.HDAC.Class.I | | | | | | |
| X.ID.200151_1.NAME.Syndecan.1.mediated. | 1.19 | 0.982 | 1.441 | 0.07533232 | 865 | 0.20925644 |
| signaling.events | | | | | | |
| X.ID.200025_1.NAME.Glypican.1.network | 1.189 | 0.98 | 1.443 | 0.079817332 | 865 | 0.21004561 |
| X.ID.100168_1.NAME.extrinsic.prothrombin. | 1.183 | 0.974 | 1.437 | 0.089596409 | 865 | 0.21694644 |
| activation.pathway | | | | | | |
| X.ID.100173_1.NAME.neuroregulin.receptor. | 1.179 | 0.974 | 1.428 | 0.091117503 | 865 | 0.21694644 |
| degredation.protein.1.controls.erbb3.receptor. | | | | | | |
| recycling | | | | | | |
| X.ID.200219_5.NAME.TGF.beta.receptor. | 1.169 | 0.965 | 1.417 | 0.11007409 | 865 | 0.24073023 |
| signaling | | | | | | |
| X.ID.200207_2.NAME.Trk.receptor.signaling. | 1.17 | 0.965 | 1.419 | 0.110735908 | 865 | 0.24073023 |
| mediated.by.PI3K.and.PLC.gamma | | | | | | |
| X.ID.100056_1.NAME.rac1.cell.motility. | 1.16 | 0.957 | 1.406 | 0.130596576 | 865 | 0.2720762 |
| signaling.pathway | | | | | | |
| X.ID.500097_1.NAME.L1CAM.interactions | 1.15 | 0.95 | 1.392 | 0.152543721 | 865 | 0.30508744 |
| X.ID.500945_1.NAME.Removal.of.DNA.patch. | 1.141 | 0.942 | 1.384 | 0.178141474 | 865 | 0.34257976 |
| containing.abasic.residue | | | | | | |
| X.ID.200187_1.NAME.Aurora.A.signaling | 1.137 | 0.939 | 1.377 | 0.186789347 | 865 | 0.3459062 |
| X.ID.100159_1.NAME.cell.cycle..g2.m. | 1.13 | 0.932 | 1.369 | 0.212880024 | 865 | 0.3801429 |
| checkpoint | | | | | | |
| X.ID.200024_1.NAME.Signaling.events. | 1.122 | 0.926 | 1.359 | 0.240797946 | 865 | 0.41434285 |
| mediated.by.HDAC.Class.III | | | | | | |
| X.ID.200165_1.NAME.Hedgehog.signaling. | 1.12 | 0.924 | 1.359 | 0.248605709 | 865 | 0.41434285 |
| events.mediated.by.Gli.proteins | | | | | | |
| X.ID.200011_1.NAME.Aurora.B.signaling | 1.11 | 0.917 | 1.344 | 0.285846316 | 865 | 0.44824191 |
| X.ID.100123_1.NAME.integrin.signaling. | 1.11 | 0.916 | 1.344 | 0.28687482 | 865 | 0.44824191 |
| pathway | | | | | | |
| X.ID.100189_1.NAME.induction.of.apoptosis. | 1.105 | 0.913 | 1.339 | 0.304168298 | 865 | 0.46086106 |
| through.dr3.and.dr4.5.death.receptors | | | | | | |
| X.ID.200144_1.NAME.PDGFR.beta.signaling. | 1.085 | 0.896 | 1.314 | 0.402128613 | 865 | 0.59136561 |
| pathway | | | | | | |
| X.ID.200128_1.NAME.Syndecan.4.mediated. | 1.08 | 0.892 | 1.308 | 0.431005839 | 865 | 0.61572263 |
| signaling.events | | | | | | |
| X.ID.100041_1.NAME.rho.cell.motility.signaling. | 1.072 | 0.883 | 1.3 | 0.482705894 | 865 | 0.66523389 |
| pathway | | | | | | |
| X.ID.100212_1.NAME.cdc25.and.chk1. | 1.069 | 0.883 | 1.295 | 0.492273081 | 865 | 0.66523389 |
| regulatory.pathway.in.response.to.dna.damage | | | | | | |
| X.ID.500100_1.NAME.Signal.transduction.by.L1 | 1.064 | 0.878 | 1.289 | 0.526495328 | 865 | 0.69275701 |
| X.ID.100152_1.NAME.inactivation.of.gsk3.by. | 1.058 | 0.873 | 1.281 | 0.564628607 | 865 | 0.72388283 |
| akt.causes.accumulation.of.b.catenin.in.alveolar. | | | | | | |
| macrophages | | | | | | |
| X.ID.500406_3.NAME.Chemokine.receptors. | 1.051 | 0.868 | 1.273 | 0.609201416 | 865 | 0.74682016 |
| bind.chemokines | | | | | | |
| X.ID.100114_1.NAME.role.of.mal.in.rho. | 1.051 | 0.868 | 1.272 | 0.612392531 | 865 | 0.74682016 |
| mediated.activation.of.srf | | | | | | |
| X.ID.100239_1.NAME.adp.ribosylation.factor | 1.042 | 0.86 | 1.262 | 0.67381999 | 865 | 0.80216665 |
| X.ID.500307_1.NAME.PECAM1.interactions | 1.031 | 0.852 | 1.249 | 0.751992857 | 865 | 0.86011002 |
| X.ID.100022_1.NAME.t.cell.receptor.signaling. | 1.03 | 0.85 | 1.247 | 0.765552387 | 865 | 0.86011002 |
| pathway | | | | | | |
| X.ID.100046_1.NAME.rb.tumor.suppressor. | 1.028 | 0.849 | 1.245 | 0.774099017 | 865 | 0.86011002 |
| checkpoint.signaling.in.response.to.dna.damage | | | | | | |
| X.ID.200031_2.NAME.E2F.transcription.factor. | 0.979 | 0.808 | 1.185 | 0.826397949 | 865 | 0.8841523 |
| network | | | | | | |
| X.ID.500652_1.NAME.Generic.Transcription. | 1.021 | 0.843 | 1.236 | 0.831103159 | 865 | 0.8841523 |
| Pathway | | | | | | |
| X.ID.200022_1.NAME.Signaling.events. | 0.986 | 0.812 | 1.196 | 0.884026332 | 865 | 0.92086076 |
| mediated.by.HDAC.Class.II | | | | | | |
| X.ID.100082_1.NAME.thrombin.signaling.and. | 1.011 | 0.834 | 1.224 | 0.914067256 | 865 | 0.93272169 |
| protease.activated.receptors | | | | | | |
| X.ID.500405_5.NAME.Peptide.ligand.binding. | 0.995 | 0.819 | 1.208 | 0.957581834 | 865 | 0.95758183 |
| receptors | | | | | | |

TABLE 17
Ovarian cancer Model E. Hazard ratios (95% CI, p values, size of the validation
cohort and q values) of patients' MDS based classification. A univariate Cox proportional
hazards model was fit to each of the top ranked subnetwork markers (n_{Breast} = 50, n_{Colon} = 75,
n_{NSCLC} = 25 and n_{Ovarian} = 50) and subsequently applied to predict patient risk score in the
validation cohort. The survival differences between the predicted groups were assessed
using Kaplan-Meier analysis.

| | | 95% CI | 95% CI | | | |
|---|---|---|---|---|---|---|
| Subnetwork module | HR | lower | upper | P | n | Q |
| X.ID.100178_1.NAME.regulation.of.eif. | 1.297 | 1.07 | 1.573 | 0.008185594 | 865 | 0.1990452 |
| 4e.and.p70s6.kinase | | | | | | |
| X.ID.200005_1.NAME.BCR.signaling. | 1.29 | 1.062 | 1.567 | 0.010226188 | 865 | 0.1990452 |
| pathway | | | | | | |
| X.ID.200048_1.NAME.Calcineurin. | 1.279 | 1.056 | 1.549 | 0.011942709 | 865 | 0.1990452 |
| regulated.NFAT.dependent.transcription. | | | | | | |
| in.lymphocytes | | | | | | |
| X.ID.200129_1.NAME.ATF.2. | 1.251 | 1.03 | 1.52 | 0.023664091 | 865 | 0.2588539 |
| transcription.factor.network | | | | | | |
| X.ID.200043_1.NAME.IL12.mediated. | 1.244 | 1.027 | 1.507 | 0.025885391 | 865 | 0.2588539 |
| signaling.events | | | | | | |
| X.ID.100185_1.NAME.regulation.of.map. | 0.815 | 0.673 | 0.988 | 0.037269305 | 865 | 0.3105775 |
| kinase.pathways.through.dual.specificity. | | | | | | |
| phosphatases | | | | | | |
| X.ID.100169_1.NAME.mets.affect.on. | 1.208 | 0.998 | 1.463 | 0.052954234 | 865 | 0.3204575 |
| macrophage.differentiation | | | | | | |
| X.ID.200122_1.NAME.Integrins.in. | 0.826 | 0.68 | 1.003 | 0.05336248 | 865 | 0.3204575 |
| angiogenesis | | | | | | |
| X.ID.200050_1.NAME.EPHB.forward. | 1.207 | 0.994 | 1.465 | 0.057682345 | 865 | 0.3204575 |
| signaling | | | | | | |
| X.ID.100113_1.NAME.mapkinase. | 1.197 | 0.984 | 1.457 | 0.072822028 | 865 | 0.3641101 |
| signaling.pathway | | | | | | |
| X.ID.200169_1.NAME.Regulation.of. | 1.169 | 0.965 | 1.417 | 0.11137119 | 865 | 0.5062327 |
| nuclear.beta.catenin.signaling.and.target. | | | | | | |
| gene.transcription | | | | | | |
| X.ID.200183_2.NAME.a6b1.and.a6b4. | 1.164 | 0.959 | 1.411 | 0.123745397 | 865 | 0.5156058 |
| Integrin.signaling | | | | | | |
| X.ID.200190_1.NAME.Class.I.PI3K. | 1.149 | 0.948 | 1.392 | 0.156668832 | 865 | 0.5638814 |
| signaling.events.mediated.by.Akt | | | | | | |
| X.ID.100252_1.NAME.agrin.in. | 1.148 | 0.948 | 1.39 | 0.157886784 | 865 | 0.5638814 |
| postsynaptic.differentiation | | | | | | |
| X.ID.100244_1.NAME.alk.in.cardiac. | 0.894 | 0.735 | 1.089 | 0.266885833 | 865 | 0.7131905 |
| myocytes | | | | | | |
| X.ID.100196_1.NAME.activation.of.csk. | 1.114 | 0.919 | 1.35 | 0.270649373 | 865 | 0.7131905 |
| by.camp.dependent.protein.kinase.inhibits. | | | | | | |
| signaling.through.the.t.cell.receptor | | | | | | |
| X.ID.100022_1.NAME.t.cell.receptor. | 0.9 | 0.743 | 1.09 | 0.279703937 | 865 | 0.7131905 |
| signaling.pathway | | | | | | |
| X.ID.200211_1.NAME.Alpha.synuclein. | 0.898 | 0.739 | 1.092 | 0.282213691 | 865 | 0.7131905 |
| signaling | | | | | | |
| X.ID.100129_1.NAME.il.2.receptor.beta. | 1.111 | 0.917 | 1.345 | 0.283203307 | 865 | 0.7131905 |
| chain.in.t.cell.activation | | | | | | |
| X.ID.100040_1.NAME.double.stranded. | 0.906 | 0.748 | 1.097 | 0.311843596 | 865 | 0.7131905 |
| rna.induced.gene.expression | | | | | | |
| X.ID.100227_2.NAME.bcr.signaling. | 1.102 | 0.908 | 1.336 | 0.326371796 | 865 | 0.7131905 |
| pathway | | | | | | |
| X.ID.100008_1.NAME.ucalpain.and. | 1.101 | 0.906 | 1.338 | 0.334821621 | 865 | 0.7131905 |
| friends.in.cell.spread | | | | | | |
| X.ID.500101_1.NAME.CHL1.interactions | 1.099 | 0.907 | 1.332 | 0.336174578 | 865 | 0.7131905 |
| X.ID.100123_1.NAME.integrin.signaling. | 1.093 | 0.901 | 1.325 | 0.368047247 | 865 | 0.7131905 |
| pathway | | | | | | |
| X.ID.200064_1.NAME.Wnt.signaling. | 1.091 | 0.901 | 1.321 | 0.374231112 | 865 | 0.7131905 |
| network | | | | | | |
| X.ID.500556_2.NAME.CDO.in. | 0.92 | 0.76 | 1.113 | 0.389808886 | 865 | 0.7131905 |
| myogenesis | | | | | | |
| X.ID.200208_2.NAME.Downstream. | 1.087 | 0.896 | 1.32 | 0.397265941 | 865 | 0.7131905 |
| signaling.in.naive.CD8..T.cells | | | | | | |
| X.ID.100056_1.NAME.rac1.cell.motility. | 0.921 | 0.76 | 1.116 | 0.399386701 | 865 | 0.7131905 |
| signaling.pathway | | | | | | |
| X.ID.100250_1.NAME.hemoglobins. | 0.922 | 0.76 | 1.119 | 0.413734178 | 865 | 0.7133348 |
| chaperone | | | | | | |
| X.ID.200102_1.NAME.FoxO.family. | 1.077 | 0.889 | 1.306 | 0.446311405 | 865 | 0.7438523 |
| signaling | | | | | | |
| X.ID.200074_1.NAME.Signaling.events. | 0.942 | 0.778 | 1.14 | 0.537063463 | 865 | 0.8268105 |
| mediated.by.TCPTP | | | | | | |
| X.ID.500150_1.NAME.Glutamate. | 0.943 | 0.779 | 1.143 | 0.551617993 | 865 | 0.8268105 |
| Neurotransmitter.Release.Cycle | | | | | | |
| X.ID.200085_1.NAME.Role.of. | 1.06 | 0.875 | 1.284 | 0.553076326 | 865 | 0.8268105 |
| Calcineurin.dependent.NFAT.signaling.in. | | | | | | |
| lymphocytes | | | | | | |
| X.ID.500128_1.NAME.Insulin.Synthesis. | 1.059 | 0.872 | 1.286 | 0.564828599 | 865 | 0.8268105 |
| and.Processing | | | | | | |
| X.ID.200065_1.NAME.TRAIL.signaling. | 1.056 | 0.872 | 1.279 | 0.578767316 | 865 | 0.8268105 |
| pathway | | | | | | |
| X.ID.100144_1.NAME.hiv.1.nef.. | 1.054 | 0.863 | 1.288 | 0.605200572 | 865 | 0.8331747 |
| negative.effector.of.fas.and.tnf | | | | | | |
| X.ID.200212_1.NAME.VEGFR3. | 1.048 | 0.865 | 1.271 | 0.6298329 | 865 | 0.8331747 |
| signaling.in.lymphatic.endothelium | | | | | | |
| X.ID.200185_1.NAME.Syndecan.2. | 1.049 | 0.863 | 1.274 | 0.633212736 | 865 | 0.8331747 |
| mediated.signaling.events | | | | | | |
| X.ID.100085_1.NAME.p38.mapk. | 1.034 | 0.854 | 1.253 | 0.730148154 | 865 | 0.9360874 |
| signaling.pathway | | | | | | |
| X.ID.500866_1.NAME.mRNA.Splicing... | 0.975 | 0.804 | 1.182 | 0.796526538 | 865 | 0.9687116 |
| Major.Pathway | | | | | | |
| X.ID.100088_2.NAME.nfkb.activation. | 0.983 | 0.812 | 1.191 | 0.86234831 | 865 | 0.9687116 |
| by.nontypeable.hemophilus.influenzae | | | | | | |
| X.ID.500652_1.NAME.Generic. | 1.016 | 0.839 | 1.232 | 0.867516536 | 865 | 0.9687116 |
| Transcription.Pathway | | | | | | |
| X.ID.200128_1.NAME.Syndecan.4. | 1.016 | 0.839 | 1.231 | 0.871085159 | 865 | 0.9687116 |
| mediated.signaling.events | | | | | | |
| X.ID.200137_1.NAME.EPHA.forward. | 1.015 | 0.838 | 1.23 | 0.875898596 | 865 | 0.9687116 |
| signaling | | | | | | |
| X.ID.200126_2.NAME.ErbB1. | 1.014 | 0.837 | 1.228 | 0.889700411 | 865 | 0.9687116 |
| downstream.signaling | | | | | | |
| X.ID.200024_1.NAME.Signaling.events. | 0.986 | 0.811 | 1.199 | 0.891214634 | 865 | 0.9687116 |
| mediated.by.HDAC.Class.III | | | | | | |
| X.ID.500655_1.NAME.Processing.of. | 0.991 | 0.818 | 1.201 | 0.926014596 | 865 | 0.9789735 |
| Capped.Intron.Containing.Pre.mRNA | | | | | | |
| X.ID.200081_2.NAME.Regulation.of. | 0.993 | 0.82 | 1.202 | 0.939814605 | 865 | 0.9789735 |
| Telomerase | | | | | | |
| X.ID.200079_1.NAME.Signaling.events. | 0.997 | 0.822 | 1.209 | 0.974386087 | 865 | 0.9942715 |
| mediated.by.HDAC.Class.I | | | | | | |
| X.ID.100221_2.NAME.role.of.egf. | 1 | 0.826 | 1.211 | 0.999369154 | 865 | 0.9993692 |
| receptor.transactivation.by.gpcrs.in. | | | | | | |
| cardiac.hypertrophy | | | | | | |

### Individual Subnetworks Directly Predict Patient Outcome

At device 10, module/pathway identification component 162 processes the subnetwork module scores, as calculated by module scoring component 154, to identify one or more dysregulated subnetwork modules. Upon identifying one or more dysregulated subnetwork modules, module/pathway identification component 162 may process the pathway records stored in datastore 144 to identify one or more biological pathway associated with the identified dysregulated subnetwork modules as dysregulated pathways.

Identifying dysregulation of particular subnetwork modules and/or pathways for specific diseases (or other phenotypes) provides targets for treatment.

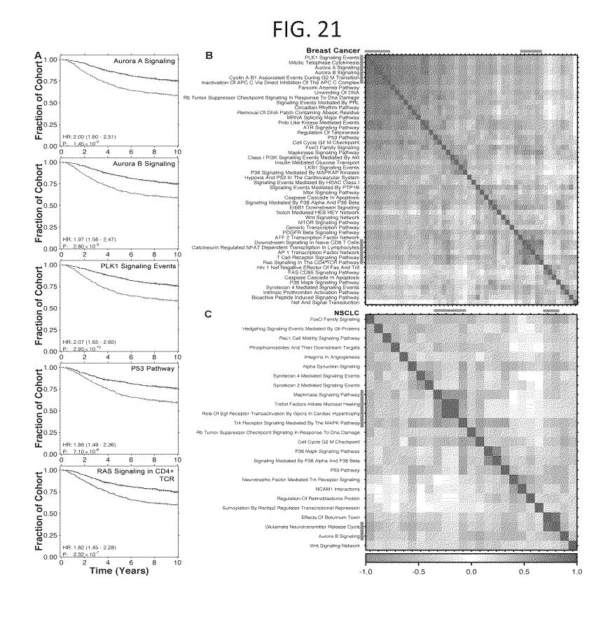

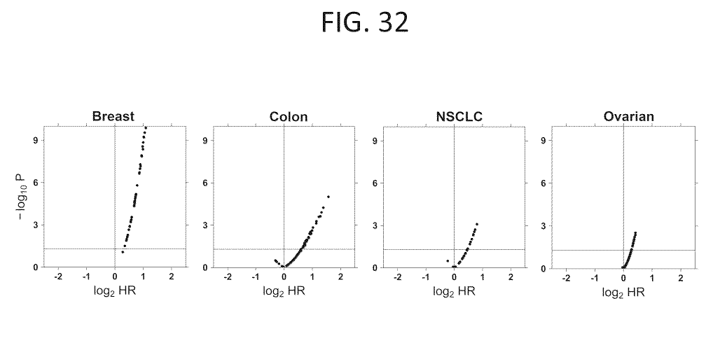

For example, by acting at the pathway level, insight can be provided about therapeutic approaches that might target an entire pathway. Subnetwork module scores are used to identify specific pathways statistically-significantly dysregulated in each disease (Methods section: Patient risk score). Survival analysis demonstrated that the subnetwork based patient risk scores were prognostic indicators of patient outcome in each tumour type (FIGS. 21A, 32, Tables 14-17). Well-known oncogenic pathways were identified, such as Aurora Kinase A and B signaling, apoptosis, DNA repair, RAS signaling, telomerase regulation and P53 activity in breast cancer [79]. Given the independent validation sets used, significant association between MDS and clinical outcome indicates the prognostic value of functionally related gene sets.

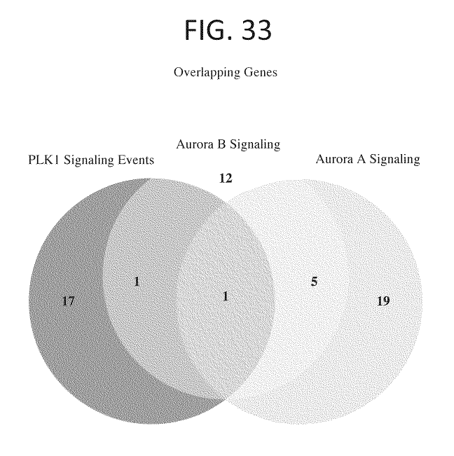

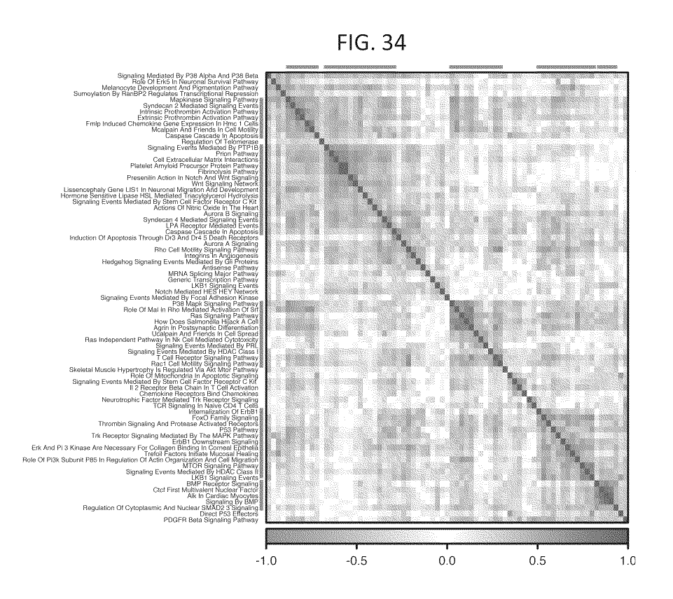

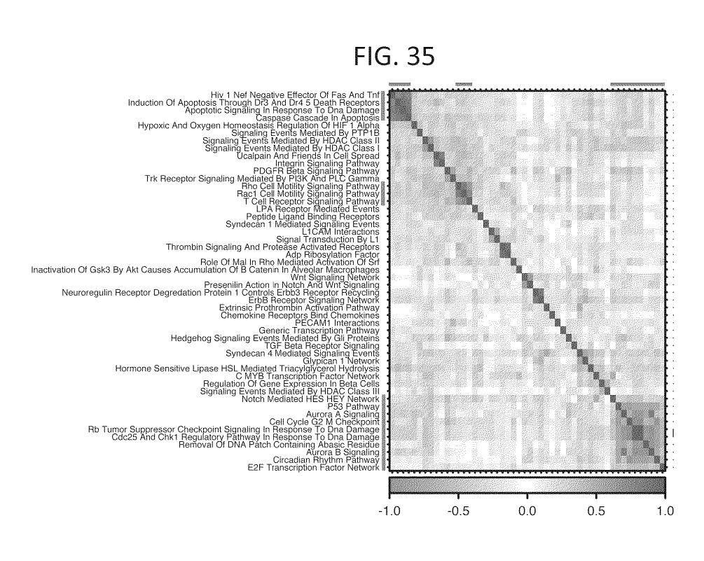

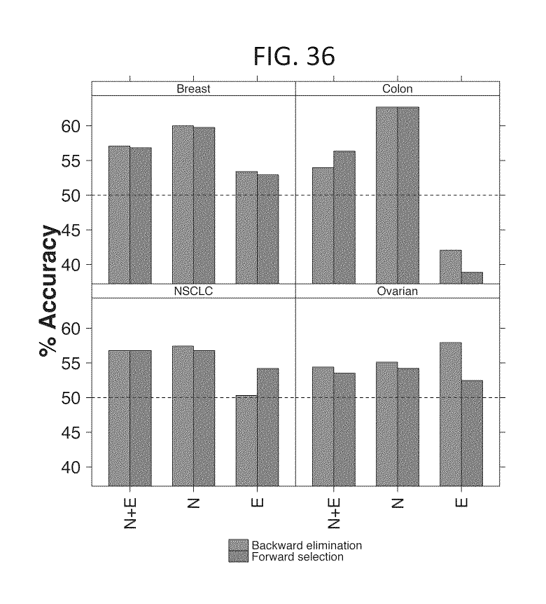

Having established that the subnetwork modules are predictive of clinical phenotype, the inter-subnetwork co-occurrence and mutual exclusivity in breast cancer (FIG. 21B) were examined. Pathways encompassing mitotic genes (PLK1, AURKA and AURKB) and their immediate interactors were both highly prognostic and tightly correlated. These subnetworks are largely disjoint, sharing only one gene in common (FIG. 33). Another noticeable cluster with consistent co-occurrence involved members of T cell receptor signaling pathways including a highly prognostic subnetwork; “RAS signaling in the CD4+ TCR” (HR=1.82, 95% Cl=1.45-2.28, p=2.32×10⁻⁷). Interestingly, this subnetwork module itself is a mediator between RAS family/GDP complex and subnetwork derived from “Calcium signaling in the CD4+ TCR” pathway. This underlines the importance of pathways that may not contain any disease associated or putative disease genes, yet possess prognostic capability. The prognostic value of the CD4+ TCR pathway asserts the immune system's role in preventing tumour progression, which is regarded as an emerging hallmark of cancer [79, 80]. Similar sets of co-occurring networks were identified in NSCLC, colon and ovarian cancers (FIGS. 21C, 34-35), demonstrating that SIMMS can identify subnetworks that are biologically relevant and functionally interpretable.

### Pan-Cancer Analysis Reveals Recurrently Dysregulated Subnetworks

Next, it was determined if specific pathways were recurrently mutated across different tumour types, in spite of the large inter-patient variability in disease presentation [69]. There were some clear similarities in subnetwork dysregulation between cancer types, with four pathways dysregulated in all types (FIG. 22A). Three of these pathways are extremely well-known for their association with cancer (P53 signaling, WNT signaling, Aurora B signaling), while the fourth (Syndecan 4 mediated signaling) is not. Subnetworks present in at least 3 tumour types were focused on (FIG. 22B), including several other well-known tumour-associated pathways such as Notch, Rb and PDGFR, along with processes widely associated with cancer such as apoptosis and G2-M cell-cycle check-points (FIG. 22B).

In addition to identifying specific subnetworks dysregulated in each disease type (e.g., each tumour type), a more general question is to quantitatively determine the similarity between different tumour types at the pathway-level. This question was addressed by sampling random sets of subnetworks, generating a prognostic model for each, and comparing the prognostic capacity of this model on each tumour type. Then million random samples of n subnetworks (where n=5, 10, 15, . . . , 250) were generated and tested their prognostic capability in the 4 tumour types. Breast and NSCLC markers showed a modest correlation (FIG. 22C; Spearman's p=0.33, p<2.2×10⁻¹⁶), indicating a fundamental similarity and presence of core underlying pathways. Most other tumour-pairs showed little correlation, but interesting differences emerged: for example colon cancers showed weak similarity to lung cancers (p=0.21) but none to breast (p=0.08) or ovarian (p=0.03).

Performance as a function of biomarker size was also analyzed (FIG. 22D). Breast and NSCLC markers showed similar profiles, but overall breast cancer markers carried higher prognostic power compared to colon, NSCLC and ovarian cancers. One explanation for this trend is the higher heterogeniety in the etiologies of these diseases as compared to breast cancer. Another is the well-defined molecular subtypes of breast cancer [81], which contrasts to the minimal overlap and poor reproducibility of molecular markers in colon [82], NSCLC [78, 83] and ovarian [84] cancers.

### Multi-Pathway Biomarkers Predict Patient Outcome

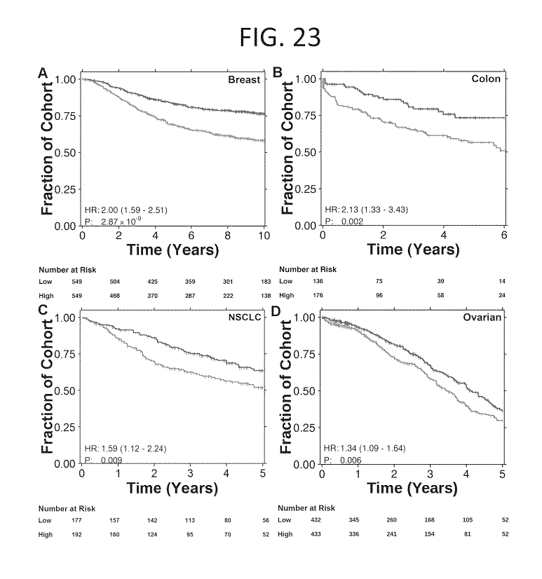

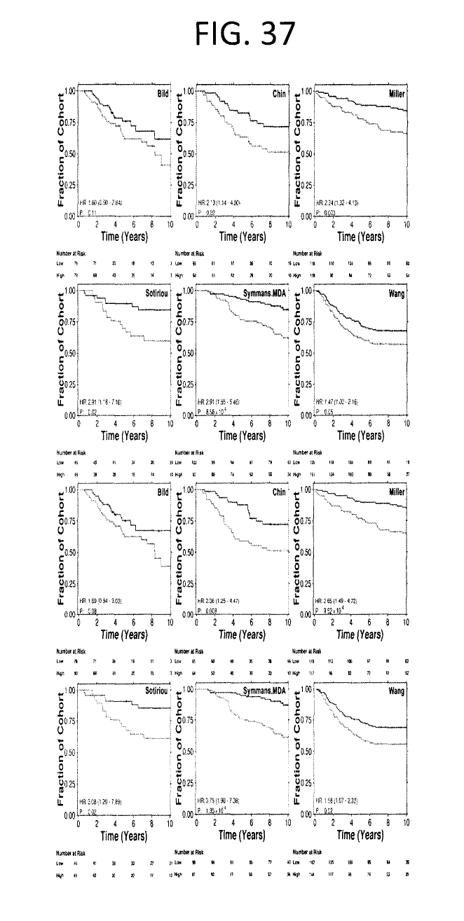

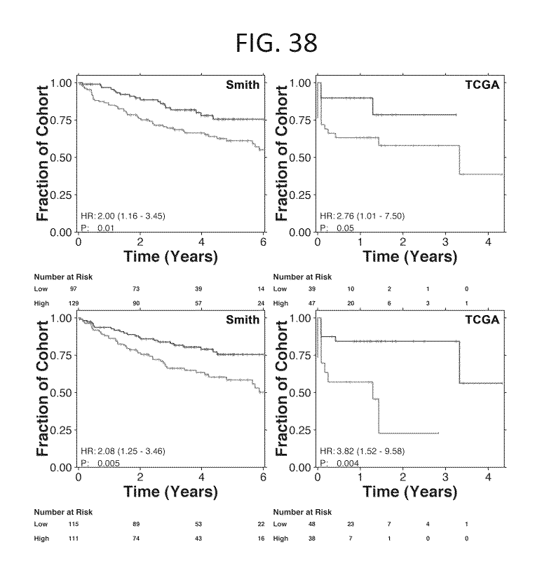

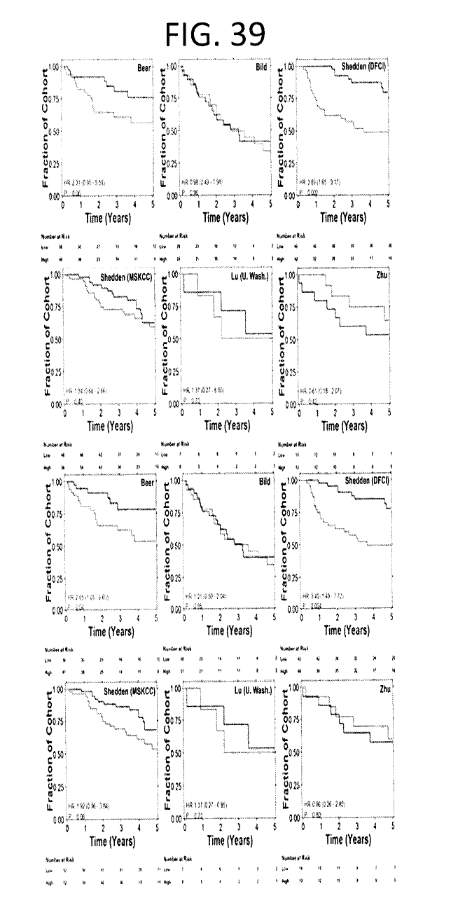

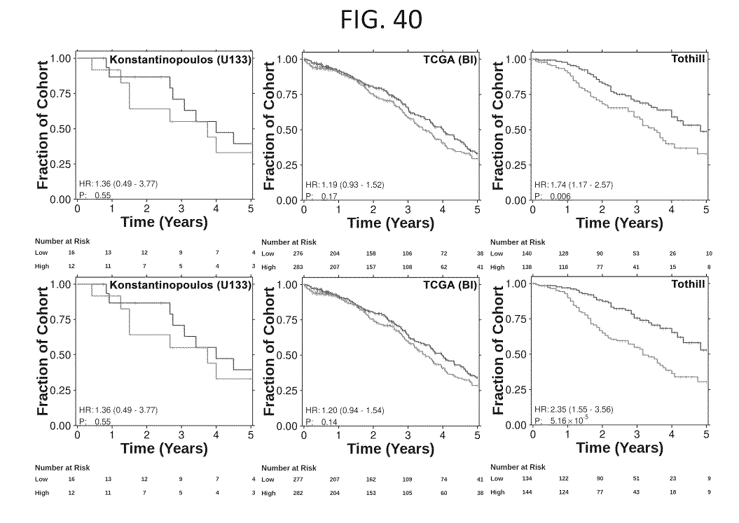

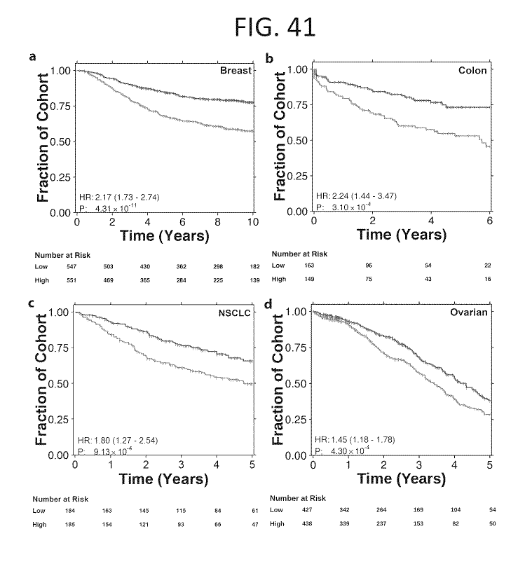

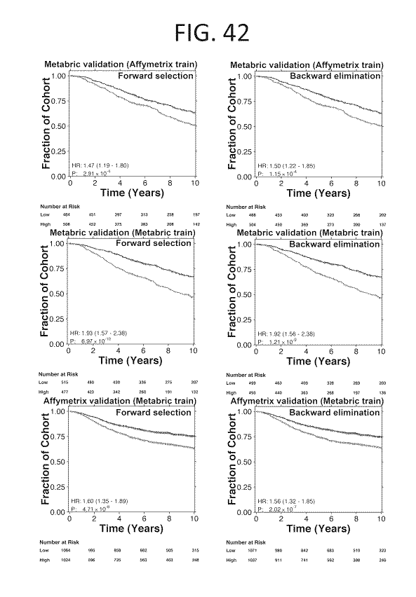

The ability of biomarker construction/pathway identification application 150 to construct clinically-use biomarkers for each of the four noted tumor types was assessed. The most optimal size of subnetworks for different tumour types was determined using permutation analysis (FIG. 22D) (n_{Breast}=50, n_{Colon}=75, n_{NSCLC}=25 and n_{Ovarian}=50). Using Model N, multivariate prognostic classifiers using forward selection were created for each tumour type in manners described above. These classifiers were employed to predict clinical outcome in independent clinical cohorts. For each tumour type, subnetwork-based biomarkers encompassing multiple pathways successfully predicted patient survival (FIGS. 23A-D, 36, Tables 18-25). Further, these results are not driven by a single cohort or study, but rather were reproducible across the vast majority of studies (FIGS. 37-40). Similarly the ability of SIMMS to generate useful biomarkers for multiple tumour-types was not a function of the feature-selection approach: multivariate analysis using backward selection yielded similar results (FIGS. 41-42, Tables 22-25).

TABLE 11
List of colon [100, 127-129] cancer studies used
for training and validation of prognostic models using
SIMMS. Studies within each cancer type were divided
into training and independent validation cohorts.

| | Patients | | | |
|---|---|---|---|---|
| | with | | | |
| | Survival | | | Analysis |
| Study | Data | Genes | Array Platform | Group |
| Jorissen et al. | 80 | 17788 | HG-U133-PLUS2 | Training |
| Loboda et al. | 125 | 15015 | Rosetta custom | Training |
| | | | human 23K array | |
| Smith et al. | 226 | 17788 | HG-U133-PLUS2 | Validation |
| TCGA | 86 | 16253 | Agilent G4502A | Validation |

TABLE 12
List of colon NSCLC [103, 114, 130-133] cancer studies
used for training and validation of prognostic models
using SIMMS. Studies within each cancer type were divided
into training and independent validation cohorts.

| | Patients | | | |
|---|---|---|---|---|
| | with | | | |
| | Survival | | | Analysis |
| Study | Data | Genes | Array Platform | Group |
| Bhattacharjee et al. | 124 | 11979 | HG-U133A | Training |
| Shedden et al. (HLM) | 79 | 11979 | HG-U133A | Training |
| Shedden et al. (MI) | 177 | 11979 | HG-U133A | Training |
| Shedden et al. (DFCI) | 82 | 11979 | HG-U133A | Validation |
| Shedden et al. | 104 | 11979 | HG-U133A | Validation |
| (MSKCC) | | | | |
| Bild et al. | 57 | 17788 | HG-U133-PLUS2 | Validation |
| Beer et al. | 86 | 5209 | H-U6800 | Validation |
| Lu et al. (Lu.Wash) | 13 | 8260 | HG-U95AV2 | Validation |
| Zhu et al. | 27 | 12146 | HG-U133A | Validation |

TABLE 13
List of ovarian [107, 114, 134-137] cancer studies
used for training and validation of prognostic models
using SIMMS. Studies within each cancer type were divided
into training and independent validation cohorts.

| | Patients | | | |
|---|---|---|---|---|
| | with | | | |
| | Survival | | | Analysis |
| Study | Data | Genes | Array Platform | Group |
| Bild et al. | 131 | 12146 | HG-U133A | Training |
| Bonome et al. | 185 | 12146 | HG-U133A | Training |
| Denkert et al. | 80 | 12146 | HG-U133A | Training |
| Konstantinopoulos | 42 | 8403 | HG-U95AV2 | Training |
| et al. (U95) | | | | |
| Konstantinopoulos | 28 | 19070 | HG-U133-PLUS2 | Validation |
| et al. (U133) | | | | |
| TCGA (Broad Inst.) | 559 | 12139 | HTHG-U133A | Validation |
| Tothill et al. | 278 | 19071 | HG-U133-PLUS2 | Validation |

TABLE 18
List of breast cancer subnetwork modules selected by the forward selection algorithm while minimising
AIC metric iteratively. Each table contains HR (95% CI), p, and coefficients of the fit using a multivariate
Cox proportional hazards model. Subnetwork modules were scored using SIMMS's Model N.

| | | 95% CI | 95% CI | | |
|---|---|---|---|---|---|
| Subnetwork module | HR | lower | upper | P | beta |
| X.ID.100113_1.NAME.mapkinase. | 1.100433243 | 0.999315973 | 1.211782214 | 0.051648714 | 0.095703959 |
| signaling.pathway | | | | | |
| X.ID.200079_1.NAME.Signaling. | 1.056302837 | 0.970851721 | 1.149275073 | 0.203139591 | 0.054774922 |
| events.mediated.by.HDAC. | | | | | |
| Class.I | | | | | |
| X.ID.100084_1.NAME.hypoxia. | 1.156324939 | 1.041229481 | 1.284142823 | 0.006622728 | 0.14524682 |
| and.p53.in.the.cardiovascular. | | | | | |
| system | | | | | |
| X.ID.200076_2.NAME.FAS.. | 1.104058981 | 1.004361324 | 1.213653099 | 0.040355867 | 0.098993371 |
| CD95..signaling.pathway | | | | | |
| X.ID.200070_3.NAME.LKB1. | 1.18455099 | 1.065712183 | 1.316641652 | 0.001690321 | 0.169363792 |
| signaling.events | | | | | |
| X.ID.200064_1.NAME.Wnt. | 1.086790426 | 0.998529333 | 1.182853012 | 0.054115885 | 0.083228789 |
| signaling.network | | | | | |
| X.ID.500377_1.NAME.Unwinding. | 0.880420294 | 0.782095725 | 0.991106164 | 0.035046463 | −0.127355879 |
| of.DNA | | | | | |
| X.ID.200006_1.NAME.Signaling. | 1.187789208 | 1.07719047 | 1.309743487 | 0.0005584 | 0.172093771 |
| events.mediated.by.PRL | | | | | |
| X.ID.500755_1.NAME.Nef.and. | 1.113976142 | 1.000428002 | 1.240411947 | 0.049095063 | 0.107935725 |
| signal.transduction | | | | | |
| X.ID.100046_1.NAME.rb.tumor. | 0.841303788 | 0.738793604 | 0.958037618 | 0.009144602 | −0.172802462 |
| suppressor.checkpoint.signaling. | | | | | |
| in.response.to.dna.damage | | | | | |
| X.ID.200129_1.NAME.ATF.2. | 1.203025255 | 1.07796001 | 1.342600607 | 0.00096557 | 0.18483943 |
| transcription.factor.network | | | | | |
| X.ID.200126_2.NAME.ErbB1. | 0.838714219 | 0.758082197 | 0.927922518 | 0.000648403 | −0.175885251 |
| downstream.signaling | | | | | |
| X.ID.200220_1.NAME.Notch. | 1.173080846 | 1.01882968 | 1.350685692 | 0.026465631 | 0.159633489 |
| mediated.HES.HEY.network | | | | | |
| X.ID.500068_1.NAME.Fanconi. | 0.84442457 | 0.717697528 | 0.993528369 | 0.041527694 | −0.169099866 |
| Anemia.pathway | | | | | |
| X.ID.500652_1.NAME.Generic. | 1.075354337 | 0.970908501 | 1.191035971 | 0.163429107 | 0.072650223 |
| Transcription.Pathway | | | | | |
| X.ID.100122_1.NAME.intrinsic. | 1.096236787 | 0.975603996 | 1.231785745 | 0.122410564 | 0.091883212 |
| prothrombin.activation.pathway | | | | | |
| X.ID.500945_1.NAME.Removal. | 1.084552526 | 0.973146537 | 1.208712292 | 0.142175334 | 0.081167483 |
| of.DNA.patch.containing. | | | | | |
| abasic.residue | | | | | |

TABLE 19
List of colon cancer subnetwork modules selected by the forward selection algorithm while minimising
AIC metric iteratively. Each table contains HR (95% CI), p, and coefficients of the fit using a multivariate
Cox proportional hazards model. Subnetwork modules were scored using SIMMS's Model N.

| | | 95% CI | 95% CI | | |
|---|---|---|---|---|---|
| Subnetwork module | HR | lower | upper | P | beta |
| X.ID.100113_1.NAME.mapkinase. | 1.060697773 | 0.996504413 | 1.129026376 | 0.064309673 | 0.058926968 |
| signaling.pathway | | | | | |
| X.ID.100106_1.NAME.role.of. | 0.997434362 | 0.84008858 | 1.184250482 | 0.97660291 | −0.002568935 |
| mitochondria.in.apoptotic.signaling | | | | | |
| X.ID.200185_1.NAME.Syndecan. | 1.126080049 | 0.989330155 | 1.28173216 | 0.072244886 | 0.118742618 |
| 2.mediated.signaling.events | | | | | |
| X.ID.200114_2.NAME.Direct.p53. | 1.295066443 | 1.047778622 | 1.600717038 | 0.016771477 | 0.258562001 |
| effectors | | | | | |
| X.ID.200081_2.NAME.Regulation. | 1.249128763 | 1.039665896 | 1.50079239 | 0.017532674 | 0.222446318 |
| of.Telomerase | | | | | |
| X.ID.200070_1.NAME.LKB1. | 1.224074759 | 1.058999498 | 1.414881706 | 0.006227321 | 0.20218526 |
| signaling.events | | | | | |
| X.ID.100129_1.NAME.il.2.receptor. | 1.27208419 | 1.027231223 | 1.575300818 | 0.027364844 | 0.24065665 |
| beta.chain.in.t.cell.activation | | | | | |
| X.ID.200012_2.NAME.LPA.receptor. | 0.845576275 | 0.707553561 | 1.010523125 | 0.065062048 | −0.167736902 |
| mediated.events | | | | | |

TABLE 20
List of NSCLC subnetwork modules selected by the forward selection algorithm while minimising AIC
metric iteratively. Each table contains HR (95% CI), p, and coefficients of the fit using a multivariate
Cox proportional hazards model. Subnetwork modules were scored using SIMMS's Model N.

| | | 95% CI | 95% CI | | |
|---|---|---|---|---|---|
| Subnetwork module | HR | lower | upper | P | beta |
| X.ID.200165_1.NAME.Hedgehog.signaling. | 1.131406481 | 0.982605474 | 1.30274119 | 0.086151003 | 0.123461532 |
| events.mediated.by.Gli.proteins | | | | | |
| X.ID.200064_1.NAME.Wnt.signaling.network | 1.229959383 | 1.077863346 | 1.403517514 | 0.00211713 | 0.206981147 |
| X.ID.100085_1.NAME.p38.mapk.signaling. | 1.195622898 | 1.050462977 | 1.360841977 | 0.006821505 | 0.178667303 |
| pathway | | | | | |
| X.ID.200211_1.NAME.Alpha.synuclein. | 1.122207437 | 1.013027592 | 1.243154225 | 0.027257085 | 0.115297671 |
| signaling | | | | | |
| X.ID.100046_1.NAME.rb.tumor.suppressor. | 1.175236487 | 0.989406092 | 1.395969575 | 0.065961471 | 0.161469393 |
| checkpoint.signaling.in.response. | | | | | |
| to.dna.damage | | | | | |
| X.ID.200145_2.NAME.Neurotrophic.factor. | 0.899064168 | 0.778071195 | 1.038871998 | 0.149067486 | −0.10640087 |
| mediated.Trk.receptor.signaling | | | | | |

TABLE 21
List of ovarian cancer subnetwork modules selected by the forward selection algorithm while minimising
AIC metric iteratively. Each table contains HR (95% CI), p, and coefficients of the fit using a multivariate
Cox proportional hazards model. Subnetwork modules were scored using SIMMS's Model N.

| | | 95% CI | 95% CI | | |
|---|---|---|---|---|---|
| Subnetwork module | HR | lower | upper | P | beta |
| X.ID.100114_1.NAME.role.of.mal. | 1.339455497 | 1.170291859 | 1.533071443 | 2.21E−05 | 0.292263186 |
| in.rho.mediated.activation.of.srf | | | | | |
| X.ID.200219_5.NAME.TGF.beta. | 1.193037922 | 0.97094367 | 1.465934151 | 0.093073932 | 0.17650293 |
| receptor.signaling | | | | | |
| X.ID.200040_1.NAME.Signaling. | 1.314926697 | 1.128941647 | 1.53155145 | 0.00043369 | 0.27378092 |
| events.mediated.by.PTP1B | | | | | |
| X.ID.100239_1.NAME.adp.ribosylation. | 1.077214206 | 0.926585716 | 1.252329304 | 0.333137871 | 0.07437827 |
| factor | | | | | |
| X.ID.500799_1.NAME.Hormone. | 0.697875861 | 0.577724852 | 0.843015002 | 0.000190408 | −0.359714041 |
| sensitive.lipase..HSL..mediated. | | | | | |
| triacylglycerol.hydrolysis | | | | | |
| X.ID.200199_1.NAME.p53.pathway | 1.14617244 | 1.031015875 | 1.274191109 | 0.011557912 | 0.136428078 |
| X.ID.500097_1.NAME.L1CAM.interactions | 1.282042317 | 1.087762699 | 1.511021205 | 0.003043687 | 0.248454367 |
| X.ID.100159_1.NAME.cell.cycle.. | 0.740081867 | 0.607610053 | 0.901435332 | 0.00277923 | −0.300994468 |
| g2.m.checkpoint | | | | | |
| X.ID.200220_1.NAME.Notch.mediated. | 1.092783091 | 0.932073699 | 1.281202211 | 0.274287752 | 0.088727737 |
| HES.HEY.network | | | | | |
| X.ID.500522_1.NAME.Regulation. | 1.263619861 | 1.051882903 | 1.517978046 | 0.012400878 | 0.233980508 |
| of.gene.expression.in.beta.cells | | | | | |
| X.ID.200207_2.NAME.Trk.receptor. | 0.728414694 | 0.57552193 | 0.921924847 | 0.008382777 | −0.316884758 |
| signaling.mediated.by.PI3K. | | | | | |
| and.PLC.gamma | | | | | |
| X.ID.200012_2.NAME.LPA.receptor. | 1.189496018 | 0.986499169 | 1.434264541 | 0.069126833 | 0.173529703 |
| mediated.events | | | | | |
| X.ID.200031_2.NAME.E2F.transcription. | 1.214816542 | 1.000005341 | 1.47577135 | 0.049993712 | 0.194593072 |
| factor.network | | | | | |
| X.ID.200022_1.NAME.Signaling. | 1.104523862 | 0.982381034 | 1.241853129 | 0.09637916 | 0.099414348 |
| events.mediated.by.HDAC.Class. | | | | | |
| II | | | | | |

TABLE 22
Performance assessment of Model N, E and N + E in respect
of breast cancer. Survival time cut-off represents the survival
time at which patients were dichotomized into naïve
low- and high-risk groups. The naïve grouping was compared
to SIMMS's predicted risk groups to compute confusion table,
sensitivity, specificity and percentage prediction accuracy.

| | Model & | | | |
|---|---|---|---|---|
| | Survival time cutoff | Sensitivity | Specificity | Accuracy |
| Backward | ‘N + E’ 8 yr | 67.55 | 50.97 | 57.07 |
| elimination | N 8 yr | 65.89 | 56.56 | 60.00 |
| | E 8 yr | 59.27 | 50.00 | 53.41 |
| Forward | ‘N + E’ 8 yr | 68.54 | 50.00 | 56.83 |
| selection | N 8 yr | 64.24 | 57.14 | 59.76 |
| | E 8 yr | 56.95 | 50.58 | 52.93 |

TABLE 23
Performance assessment of Model N, E and N + E in respect
of colon cancer. Survival time cut-off represents the survival
time at which patients were dichotomized into naïve
low- and high-risk groups. The naïve grouping was compared
to SIMMS's predicted risk groups to compute confusion table,
sensitivity, specificity and percentage prediction accuracy.

| | Model & | | | |
|---|---|---|---|---|
| | Survival time cutoff | Sensitivity | Specificity | Accuracy |
| Backward | ‘N + E’ 6 yr | 46.59 | 71.05 | 53.97 |
| elimination | N 6 yr | 64.72 | 57.89 | 62.7 |
| | E 6 yr | 34.09 | 60.53 | 42.06 |
| Forward | ‘N + E’ 6 yr | 52.27 | 65.79 | 56.35 |
| selection | N 6 yr | 73.86 | 36.84 | 62.70 |
| | E 6 yr | 36.36 | 44.74 | 38.89 |

TABLE 24
Performance assessment of Model N, E and N + E in respect
of NSCLC. Survival time cut-off represents the survival time
at which patients were dichotomized into naïve low-
and high-risk groups. The naïve grouping was compared
to SIMMS's predicted risk groups to compute confusion table,
sensitivity, specificity and percentage prediction accuracy.

| | Model & | | | |
|---|---|---|---|---|
| | Survival time cutoff | Sensitivity | Specificity | Accuracy |
| Backward | ‘N + E’ 3 yr | 55.96 | 57.21 | 56.77 |
| elimination | N 3 yr | 63.30 | 54.23 | 57.42 |
| | E 3 yr | 43.12 | 54.23 | 50.32 |
| Forward | ‘N + E’ 3 yr | 55.96 | 57.21 | 56.77 |
| selection | N 3 yr | 62.39 | 53.73 | 56.77 |
| | E 3 yr | 43.12 | 60.20 | 54.19 |

TABLE 25
Performance assessment of Model N, E and N + E in respect
of ovarian cancer. Survival time cut-off represents the survival
time at which patients were dichotomized into naïve
low- and high-risk groups. The naïve grouping was compared
to SIMMS's predicted risk groups to compute confusion table,
sensitivity, specificity and percentage prediction accuracy.

| | Model & | | | |
|---|---|---|---|---|
| | Survival time | | | |
| | cutoff | Sensitivity | Specificity | Accuracy |
| Backward | ‘N + E’ 3 yr | 57.3705179 | 52.0504732 | 54.4014085 |
| elimination | N 3 yr | 58.5657371 | 52.3659306 | 55.1056338 |
| | E 3 yr | 59.3625498 | 56.7823344 | 57.9225352 |
| Forward | ‘N + E’ 3 yr | 60.5577689 | 47.9495268 | 53.5211268 |
| selection | N 3 yr | 56.9721116 | 52.0504732 | 54.2253521 |
| | E 3 yr | 49.8007968 | 54.5741325 | 52.4647887 |

### Inter-Platform Validation of SIMMS

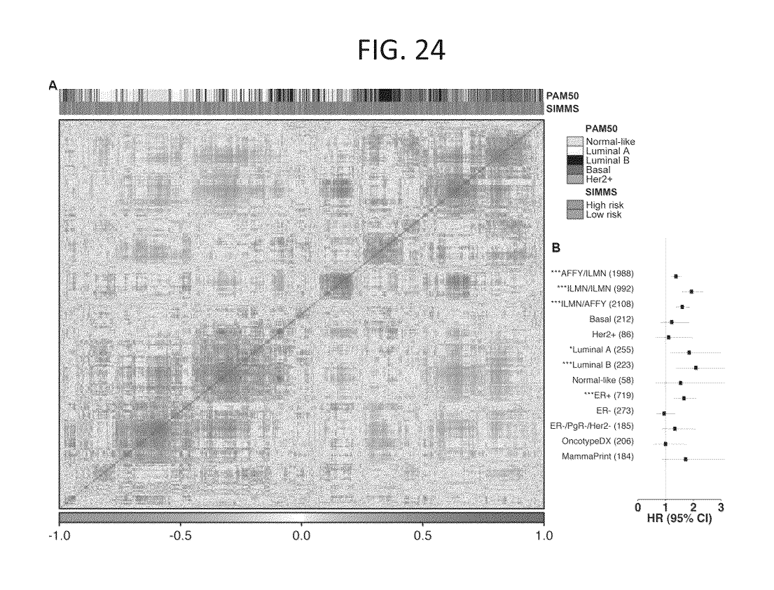

Because SIMMS operates at the level of pathways, it is robust to changes in the genomics platform. The Metabric clinical cohort of 1,988 patient profiles generated using IIlumina microarrays was used to demonstrate this flexibility [85]. The 50-subnetwork breast cancer classifier generated using Affymetrix microarrays (FIG. 24A) successfully validated in the IIlumina-based Metabric cohort (FIG. 24B, AFFY/ILMN row). Further, we used SIMMS to train a classifier on half the Metabric patients (n=996). This classifier not only validated in the other half of the Metabric cohort (FIG. 24B, ILMN/ILMN row; HR=1.93, p=6.97×10⁻¹⁰), but also in the Affymetrix datasets (FIG. 24B, ILMN/AFFY row; FIG. 42). Taken together these results indicate that, although platform changes introduce noise, SIMMS as implemented in application 150 can flexibly use and integrate data from multiple platforms.

Comparison with Existing Pan-Cancer Prognostic Biomarkers

To demonstrate the clinical utility of the biomarkers generated by SIMMS, as implemented in application 150, we conducted coherent performance comparison with previously published colon, NSCLC and ovarian cancer markers. The performance of SIMMS's identified markers was highly competitive and reproducible across a panel of independent patient studies. SIMMS produced the best prognostic marker for colon cancer by a wide margin, and was tied for the best lung and ovarian cancer markers (Table 26). Of note, each of the 15 other biomarkers evaluated used an entirely separate methodology. Overall, these results indicate that functionally-derived subnetworks have excellent prognostic capability, and can be used to identify new biomarkers across a range of human diseases.

TABLE 26
Comparison of colon, NSCLC and ovarian cancer prognostic biomarkers with the SIMMS's identified prognostic
markers. Cox model HR (95% CI) and p values (Wald-test or Logrank-test) are shown for all the models. Only
p value is reported when the HR (95% CI) was not available in the original study. Comparisons were limited
to those studies that were treated as validation cohorts by both previously published biomarkers and SIMMS
except for Smith et al. colon cancer dataset, which was partly used as the training set in the original
biomarker while completely used as a validation set by the SIMMS colon cancer classifier.

| | Validation datasets | | | |
|---|---|---|---|---|
| Colon cancer markers | Smith et al. | TCGA | | |
| SIMMS Model N (FS) | HR = 2.00 (1.16- | HR = 2.76 (1.01- | | |
| | 3.45), p = 0.01 | 7.50), p = 0.05 | | |
| SIMMS Model N (BE) | HR = 2.08 (1.25- | HR = 3.82 (1.52- | | |
| | 3.46), p = 0.005 | 9.58), p = 0.004 | | |
| Oh et al. (CCP) | p = 0.032 | | | |
| Smith et al. | HR = 1.85 (1.07- | HR = 1.39 (0.61- | | |
| | 3.21), p = 0.03 | 3.17), p = 0.44 | | |
| NSCLC markers | Beer et al. | Bild et al.¹ | Shedden et al. (DFCI) | Shedden et al. (MSKCC) |
| SIMMS Model N (FS) | HR = 2.31 (0.95- | HR = 0.98 (0.49- | HR = 3.89 (1.65- | HR = 1.34 (0.68- |
| | 5.59), p = 0.06 | 1.98), p = 0.96 | 9.17), p = 0.002 | 2.66), p = 0.40 |
| SIMMS Model N (BE) | HR = 2.65 (1.05- | HR = 1.01 (0.50- | HR = 3.40 (1.49- | HR = 1.92 (0.96- |
| | 6.69), p = 0.04 | 2.04), p = 0.98 | 7.72), p = 0.004 | 3.84), p = 0.06 |
| Boutros et al. | | HR = 3.3, p = 0.002 | HR = 0.63 (0.22- | HR = 2.04 (0.97- |
| | | | 1.78), p = 0.38 | 4.26), p = 0.06 |
| Chen et al. | p = 0.06 | | | |
| Lau et al. | HR = 1.91 (0.82- | HR = 2.5 (1.40- | HR = 1.36 (0.60- | HR = 1.88 (0.94- |
| | 4.46), p = 0.14 | 4.60), p = 0.004 | 3.05), p = 0.46 | 3.77), p = 0.08 |
| Shedden et al. (C) | | | HR = 1.07 (0.45- | HR = 1.74 (0.87- |
| | | | 2.56), p = 0.878 | 3.47), p = 0.111 |
| Shedden et al. (E) | | | HR = 0.53 (0.18- | HR = 1.44 (0.71- |
| | | | 1.56), p = 0.239 | 2.89), p = 0.301 |
| Shedden et al. (F) | | | HR = 0.98 (0.46- | HR = 2.65 (1.32- |
| | | | 2.08), p = 0.947 | 5.33), p = 0.005 |
| Shedden et al. (G) | | | HR = 1.13 (0.52- | HR = 3.19 (1.50- |
| | | | 2.46), p = 0.751 | 6.78), p = 0.002 |
| Ovarian cancer markers | TCGA | Tothill et al. | | |
| SIMMS Model N (FS) | HR = 1.19 (0.93- | HR = 1.74 (1.17- | | |
| | 1.52), p = 0.17 | 2.57), p = 0.006 | | |
| SIMMS Model N (BE) | HR = 1.20 (0.94- | HR = 2.35 (1.55- | | |
| | 1.54), p = 0.14 | 3.56), p = 5.16 × 10⁻⁵ | | |
| Yoshihara et al. | HR = 1.68 (1.20- | | | |
| | 2.32), p = 0.003 | | | |
| TCGA | | p = 8 × 10⁻⁵ | | |
| Mankoo et al. | | HR = 2.06 (1.11- | | |
| | | 3.30), p = 0.014 | | |
| Wu & Stein | HR = 1.33 (1.04- | HR = 2.43 (1.06- | | |
| | 1.69), p = 0.021 | 5.55), p = 0.036 | | |

¹The validity of this dataset has been much criticised in the literature, with several studies being retracted (PMIDs: 17057710 and 16899777)
Shedden et al. (C, E, F and G) refer to different classifiers trained on gene expression profiles only

To further establish the clinical utility of SIMMS's classifications, we tested for synergy between SIMMS-predicted risk groups and the intrinsic breast cancer subtypes [81] using the Metabric cohort. The prognostic model created on the Metabric training cohort yielded risk-groups with in agreement with the PAM50 intrinsic subtypes (FIG. 24A; F-measure=0.70). The cluster analysis affirmed that the SIMMS identified low-risk group corresponds to the Luminal-A and Normal-like breast cancers, which are bona fide good prognosis subtypes. Likewise, the SIMMS proposed high-risk group largely represented Basal, Her2-positive and Luminal-B patients, which are regarded as poor prognosis subtypes.

However SIMMS can assist in the improved clinical management of breast cancer beyond simply subtyping them. For example, the majority of Basal-like tumours are triple negatives (ER-, PgR-, and Her2-) and vice versa, yet these are heterogeneous diseases with subgroups of patients having differential response to neo-adjuvant therapy [86]. Hence, molecular biomarkers are urgently needed for better management of patient subgroups that do not respond to current therapeutic regimes. To identify such biomarkers, we created subtype-specific SIMMS classifiers for breast cancer subgroups. Despite greatly reduced sample-sizes, SIMMS's classifiers successfully stratified the most heterogeneous groups (i.e. luminal A, luminal B and ER-positive [87]) into good and poor prognosis sub-groups (FIG. 24B), and generated classifiers with the correct trend for other sub-groups.

To further demonstrate clinical utility, SIMMS's classifier was directly compared to two clinically-approved breast cancer biomarkers, Oncotype DX [88] and MammaPrint [89], in 7 independent validation cohorts. Each validation patient was classified using both these clinically-approved biomarkers and the SIMMS-trained breast-cancer classifier created using forward selection (FIG. 23A). We assessed the ability of each biomarker to stratify patients into groups with differential survival using Cox proportional hazards modeling and the Wald test (null hypothesis: HR=1.0). Across the 7 validation cohorts, the SIMMS-derived biomarker yielded the most statistically significant predictions of differential survival in 5 cohorts, while the clinically-used Oncotype DX and MammaPrint biomarkers each performed best in only one (Table 8).

### General, Multimodal Biomarkers

Large-scale disease-specific initiatives are rapidly generating matched genomic, transcriptomic and epigenomic profiling on large cohorts, with detailed clinical annotation [90]. Systematic integration of such data remains challenging, but offers the prospect for enhanced biomarker accuracy. We applied SIMMS to the Metabric dataset to combine copy number aberration (CNA) and mRNA abundance data. The integrated data yielded improved prediction relative to either data-type alone (FIGS. 25A-C). Similarly multimodal prognostic models were created using the ovarian cancer TCGA dataset [68] using matched CNA, mRNA and DNA methylation profiles (FIG. 25D). Thus SIMMS, as for example implemented by biomarker construction/pathway identification application 150 can integrate multiple molecular data types into pathway-based biomarkers.

Such data types may include data reflecting aberration, epigenomic aberration, transcriptomic aberration, proteomic aberration, and metabolic aberration, and more particularly data reflecting somatic point mutation, small indel, mRNA abundance, somatic or germline copy-number status, somatic or germline genomic rearrangements, metabolite abundance, protein abundance, and DNA methylation.

It will be appreciated that any device exemplified herein that executes instructions may include or otherwise have access to computer readable media such as storage media, computer storage media, or data storage devices (removable and/or non-removable) such as, for example, magnetic disks, optical disks, tape, and other forms of computer readable media. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD), blue-ray disks, or other optical storage, magnetic cassettes, magnetic tape, magnetic disk storage or other magnetic storage devices, or any other medium which can be used to store the desired information and which can be accessed by an application, module, or both. Any application or component herein described may be implemented using computer readable/executable instructions that may be stored or otherwise held by such computer readable media.

Furthermore, the described embodiments are capable of being distributed in a computer program product including a physical, non-transitory computer readable medium that bears computer-executable instructions for one or more processors. The medium may be provided in various forms, including one or more diskettes, compact disks, tapes, chips, magnetic and electronic storage media, volatile memory, non-volatile memory and the like. Non-transitory computer-readable media may include all computer-readable media, with the exception being a transitory, propagating signal. The term non-transitory is not intended to exclude computer readable media such as primary memory, volatile memory, RAM and so on, where the data stored thereon may only be temporarily stored. The computer useable instructions may also be in various forms, including compiled and non-compiled code.

It will be appreciated that numerous specific details are set forth in order to provide a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way, but rather as merely describing implementation of the various embodiments described herein. All references herein, including in the following Appendices and Reference List, are hereby incorporated by reference.

### REFERENCES

1. Abe O, Abe R, Enomoto K et al. Effects of chemotherapy and hormonal therapy for early breast cancer on recurrence and 15-year survival: an overview of the randomised trials. Lancet 2005; 365(9472):1687-1717.
2. Dowsett M, Cuzick J, Ingle J et al. Meta-Analysis of Breast Cancer Outcomes in Adjuvant Trials of Aromatase Inhibitors Versus Tamoxifen. Journal of Clinical Oncology 2010; 28(3):509-518.
3. Bartlett J, Canney P, Campbell A et al. Selecting breast cancer patients for chemotherapy: the opening of the UK OPTIMA trial. Clin Oncol (R Coll Radiol) 2013; 25(2):109-116.
4. Cook N R. Use and Misuse of the Receiver Operating Characteristic Curve in Risk Prediction. Circulation 2007; 115(7):928-935.
5. Sotiriou C, Wirapati P, Loi S et al. Comprehensive analysis integrating both clinicopathological and gene expression data in more than 1,500 samples: Proliferation captured by gene expression grade index appears to be the strongest prognostic factor in breast cancer (BC). Journal of Clinical Oncology 2006; 24(18):4S.
6. Afentakis M, Dowsett M, Sestak I et al. Immunohistochemical BAG1 expression improves the estimation of residual risk by IHC4 in postmenopausal patients treated with anastrazole or tamoxifen: a TransATAC study. Breast Cancer Res Treat 2013; 140(2):253-262.
7. Cuzick J, Dowsett M, Pineda S et al. Prognostic Value of a Combined Estrogen Receptor, Progesterone Receptor, Ki-67, and Human Epidermal Growth Factor Receptor 2 Immunohistochemical Score and Comparison With the Genomic Health Recurrence Score in Early. Breast Cancer. Journal of Clinical Oncology 2011; 29(32):4273-4278.
8. Ciriello G, Miller M L, Aksoy B A, Senbabaoglu Y, Schultz N, Sander C. Emerging landscape of oncogenic signatures across human cancers. Nat Genet 2013; 45(10):1127-1133.
9. Stephens P J, Tarpey P S, Davies H et al. The landscape of cancer genes and mutational processes in breast cancer. Nature 2012; 486(7403):400-404.
10. Loi S, Haibe-Kains B, Majjaj S et al. PIK3CA mutations associated with gene signature of low mTORC1 signaling and better outcomes in estrogen receptor-positive breast cancer. Proceedings of the National Academy of Sciences of the United States of America 2010; 107(22):10208-10213.
11. Loi S, Haibe-Kains B, Lallemand F et al. Pik3Ca, Akt1 Mutation and Her2 Amplification Gene Signatures (Gs) Suggest Predominantly Negative Feedback Inhibition of Pi3K/Akt Pathway in Human Breast Cancer (Bc). Annals of Oncology 2009; 20:45.
12. Sotiriou C, Loi S, Haibe-Kains B et al. PIK3CA mutation-associated gene expression signature correlates with deactivation of the PI3K pathway and predicts benefit to endocrine therapy in high-risk ER plus (luminal B) breast cancers (BC). Proceedings of the American Association for Cancer Research Annual Meeting 2009; 50:456.
13. Sabine V S, Crozier C, Brookes C L et al. Mutational analysis of PI3K/AKT Signalling Pathway in Tamoxifen Exemestane Adjuvant Multinational (TEAM) pathology study. Journal of Clinical Oncology 2014.
14. http://cancer.sanger.ac.uk/cancergenome/projects/cosmic/15.
15. Beaver J A, Park B H. The BOLERO-2 trial: the addition of everolimus to exemestane in the treatment of postmenopausal hormone receptor-positive advanced breast cancer. Future Oncol 2012; 8(6):651-657.
16. Gao Q, Patani N, Dunbier A K et al. Effect of Aromatase Inhibition on Functional Gene Modules in Estrogen ReceptorGçôPositive Breast Cancer and Their Relationship with Antiproliferative Response. Clin Cancer Res 2014; 20(9):2485-2494.
17. Beaver J A, Gustin J P, Yi K H et al. PIK3CA and AKT1 Mutations Have Distinct Effects on Sensitivity to Targeted Pathway Inhibitors in an Isogenic Luminal Breast Cancer Model System. Clin Cancer Res 2013; 19(19):5413-5422.
18. Janku F, Wheler J J, Naing A et al. PIK3CA Mutation H1047R Is Associated with Response to PI3K/AKT/mTOR Signaling Pathway Inhibitors in Early-Phase Clinical Trials. Cancer Res 2013; 73(1):276-284.
19. Arnedos M, Scott V, Job B et al. Array CGH and PIK3CA/AKT1 mutations to drive patients to specific targeted agents: A clinical experience in 108 patients with metastatic breast cancer. European journal of cancer (Oxford, England: 1990) 48[15], 2293-2299. 1-10-2012.
20. van de Velde C J H, Putter H, Seynaeve C et al. Results of the first planned analysis of the TEAM (Tamoxifen and exemestane adjuvant multinational) trial in post menopausal patients with hormone-sensitive early breast cancer. Submitted 2009.
21. van de Velde C J H, Rea D, Seynaeve C et al. Adjuvant tamoxifen and exemestane in early breast cancer (TEAM): a randomised phase 3 trial. Lancet 2011; 377(9762):321-331.
22. Bartlett J M S, Bloom K J, Piper T et al. Mammostrat as an Immunohistochemical Multigene Assay for Prediction of Early Relapse Risk in the Tamoxifen Versus Exemestane Adjuvant Multicenter Trial Pathology Study. Journal of Clinical Oncology 2012; 30(36):4477-4484.
23. Bartlett J M S, Brookes C L, Robson T et al. Estrogen Receptor and Progesterone Receptor As Predictive Biomarkers of Response to Endocrine Therapy: A Prospectively Powered Pathology Study in the Tamoxifen and Exemestane Adjuvant Multinational Trial. Journal of Clinical Oncology 2011; 29(12):1531-1538.
24. Bartlett J M S. Biomarkers and patient selection for PIK3inase/AKT/mTOR targeted therapies: Current status and future directions. Clinical Breast Cancer 2010.
25. Bartlett J M S, Going J J, Mallon E A et al. Evaluating HER2 amplification and overexpression in breast cancer. Journal of Pathology 2001; 195(4):422-428.
26. Waggott D, Chu K, Yin S, Wouters B G, Liu F F, Boutros P C. NanoStringNorm: an extensible R package for the pre-processing of NanoString mRNA and miRNA data. Bioinformatics 2012; 28(11):1546-1548.
27. Reeves J R, Going J J, Smith G, Cooke T G, Ozanne B W, Stanton P D. Quantitative radioimmunohistochemical measurements of p185(erbB-2) in frozen tissue sections. J Histochem Cytochem 1996; 44:1251-1259.
28. Wolff A C, Hammond M E, Hicks D G et al. Recommendations for Human Epidermal Growth Factor Receptor 2 Testing in Breast Cancer: American Society of Clinical Oncology/College of American Pathologists Clinical Practice Guideline Update. Journal of Clinical Oncology 2013.
29. Christiansen J, Bartlett J M, Gustayson M et al. Validation of IHC4 algorithms for prediction of risk of recurrence in early breast cancer using both conventional and quantitative IHC approaches. Journal of Clinical Oncology 2012; 30(No 15_suppl).
30. Yarden Y, Pines G. The ERBB network: at last, cancer therapy meets systems biology. Nat Rev Cancer 2012; 12(8):553-563.
31. Tovey S M, Witton C J, Bartlett J M S, Stanton P D, Reeves J R, Cooke T G. Outcome and human epidermal growth factor receptor (HER) 1-4 status in invasive breast carcinomas with proliferation indices evaluated by bromodeoxyuridine labelling. Breast Cancer Res 2004; 6(3):R246-R251.
32. Witton C J, Reeves J R, Going J J, Cooke T G, Bartlett J M S. Expression of the HERI-4 family of receptor tyrosine kinases in breast cancer. Journal of Pathology 2003; 200(3):290-297.
33. Quintayo M A, Munro A F, Thomas J et al. GSK3beta and cyclin D1 expression predicts outcome in early breast cancer patients. Breast Cancer Res Treat 2012; 136(1):161-168.
34. Kirkegaard T, Nielsen K V, Jensen L B et al. Genetic alterations of CCND1 and EMSY in breast cancers. Histopathology 2008; 52(6):698-705.
35. Lundgren K, Brown M, Pineda S et al. Effects of cyclin D1 gene amplification and protein expression on time to recurrence in postmenopausal breast cancer patients treated with anastrozole or tamoxifen: A TransATAC study. Breast Cancer Res 2012; 14(2):R57.
36. Kirkegaard T, Witton C J, Edwards J et al. Molecular alterations in AKT1, AKT2 and AKT3 detected in breast and prostatic cancer by FISH. Histopathology 2010; 56(2):203-211.
37. Kirkegaard T, Witton C J, McGlynn L M et al. AKT activation predicts outcome in breast cancer patients treated with tamoxifen. Journal of Pathology 2005; 207(2):139-146.
38. Perou C M, Sorlie T, Eisen M B et al. Molecular portraits of human breast tumours. Nature 2000; 406(6797):747-752.
39. Paik S, Shak S, Tang G et al. A multigene assay to predict recurrence of tamoxifen-treated, node-negative breast cancer. New Engl J Med 2004; 351(27):2817-2826.
40. Loi S, Michiels S, Baselga J et al. PIK3CA genotype and a PIK3CA mutation-related gene signature and response to everolimus and letrozole in estrogen receptor positive breast cancer. PLoS One 2013; 8(1):e53292.
41. Schemper M, Smith T L. A note on quantifying follow-up in studies of failure time. Control Clin Trials 1996; 17(4):343-346.
42. Cuzick J, Dowsett M, Wale C et al. Prognostic Value of a Combined ER, PgR, Ki67, HER2 Immunohistochemical (IHC4) Score and Comparison with the GHI Recurrence Score —Results from TransATAC. Cancer Res 2009; 69(24):5035.
43. de Bono J S, Ashworth A: Translating cancer research into targeted therapeutics. Nature 2010, 467:543-549.
44. Galvan A, loannidis J P, Dragani T A: Beyond genome-wide association studies: genetic heterogeneity and individual predisposition to cancer. Trends in genetics: TIG 2010, 26:132-141.
45. Veltman J A, Brunner H G: De novo mutations in human genetic disease. Nature reviews Genetics 2012, 13:565-575.
46. McClellan J, King M C: Genetic heterogeneity in human disease. Cell 2010, 141:210-217.
47. Kratz J R, He J, Van Den Eeden S K, Zhu Z H, Gao W, Pham P T, Mulvihill M S, Ziaei F, Zhang H, Su B, et al: A practical molecular assay to predict survival in resected non-squamous, non-small-cell lung cancer: development and international validation studies. Lancet 2012, 379:823-832.
48. Maycox P R, Kelly F, Taylor A, Bates S, Reid J, Logendra R, Barnes M R, Larminie C, Jones N, Lennon M, et al: Analysis of gene expression in two large schizophrenia cohorts identifies multiple changes associated with nerve terminal function. Molecular psychiatry 2009, 14:1083-1094.
49. Ein-Dor L, Zuk O, Domany E: Thousands of samples are needed to generate a robust gene list for predicting outcome in cancer. Proc Natl Acad Sci USA 2006, 103:5923-5928.
50. The Cancer Genome Atlas Research Network: Comprehensive molecular characterization of human colon and rectal cancer. Nature 2012, 487:330-337.
51. Chuang H Y, Lee E, Liu Y T, Lee D, Ideker T: Network-based classification of breast cancer metastasis. Mol Syst Biol 2007, 3:140.
52. Frey B J, Dueck D: Clustering by passing messages between data points. Science 2007, 315:972-976.
53. Gatza M L, Lucas J E, Barry W T, Kim J W, Wang Q, Crawford M D, Datto M B, Kelley M, Mathey-Prevot B, Potti A, Nevins J R: A pathway-based classification of human breast cancer. Proc Natl Acad Sci USA 2010, 107:6994-6999.
54. Jonsson P F, Cayenne T, Zicha D, Bates P A: Cluster analysis of networks generated through homology: automatic identification of important protein communities involved in cancer metastasis. BMC Bioinformatics 2006, 7:2.
55. Platzer A, Perco P, Lukas A, Mayer B: Characterization of protein-interaction networks in tumors. BMC Bioinformatics 2007, 8:224.
56. Pujana M A, Han J D, Starita L M, Stevens K N, Tewari M, Ahn J S, Rennert G, Moreno V, Kirchhoff T, Gold B, et al: Network modeling links breast cancer susceptibility and centrosome dysfunction. Nat Genet 2007, 39:1338-1349.
57. Rambaldi D, Giorgi F M, Capuani F, Ciliberto A, Ciccarelli F D: Low duplicability and network fragility of cancer genes. Trends Genet 2008, 24:427-430.
58. Taylor I W, Linding R, Warde-Farley D, Liu Y, Pesquita C, Faria D, Bull S, Pawson T, Morris Q, Wrana J L: Dynamic modularity in protein interaction networks predicts breast cancer outcome. Nat Biotechnol 2009, 27:199-204.
59. Bild A H, Yao G, Chang J T, Wang Q, Potti A, Chasse D, Joshi M B, Harpole D, Lancaster J M, Berchuck A, et al: Oncogenic pathway signatures in human cancers as a guide to targeted therapies. Nature 2006, 439:353-357.
60. Vaske C J, Benz S C, Sanborn J Z, Earl D, Szeto C, Zhu J, Haussler D, Stuart J M: Inference of patient-specific pathway activities from multi-dimensional cancer genomics data using PARADIGM. Bioinformatics 2010, 26:i237-245.
61. Drier Y, Sheffer M, Domany E: Pathway-based personalized analysis of cancer. Proceedings of the National Academy of Sciences of the United States of America 2013.
62. Subramanian J, Simon R: Gene expression-based prognostic signatures in lung cancer: ready for clinical use? Journal of the National Cancer Institute 2010, 102:464-474.
63. Bachtiary B, Boutros P C, Pintilie M, Shi W, Bastianutto C, Li J H, Schwock J, Zhang W, Penn L Z, Jurisica I, et al: Gene expression profiling in cervical cancer: an exploration of intratumor heterogeneity. Clin Cancer Res 2006, 12:5632-5640.
64. Gerlinger M, Rowan A J, Horswell S, Larkin J, Endesfelder D, Gronroos E, Martinez P, Matthews N, Stewart A, Tarpey P, et al: Intratumor heterogeneity and branched evolution revealed by multiregion sequencing. The New England journal of medicine 2012, 366:883-892.
65. Sotiriou C, Wirapati P, Loi S, Harris A, Fox S, Smeds J, Nordgren H, Farmer P, Praz V, Haibe-Kains B, et al: Gene expression profiling in breast cancer: understanding the molecular basis of histologic grade to improve prognosis. J Natl Cancer Inst 2006, 98:262-272.
66. Musgrove E A, Sutherland R L: Biological determinants of endocrine resistance in breast cancer. Nature reviews Cancer 2009, 9:631-643.
67. The Cancer Genome Atlas Research Network: Comprehensive genomic characterization defines human glioblastoma genes and core pathways. Nature 2008, 455:1061-1068.
68. The Cancer Genome Atlas Research Network: Integrated genomic analyses of ovarian carcinoma. Nature 2011, 474:609-615.
69. Vogelstein B, Kinzler K W: Cancer genes and the pathways they control. Nature medicine 2004, 10:789-799.
70. Irizarry R A, Hobbs B, Collin F, Beazer-Barclay Y D, Antonellis K J, Scherf U, Speed T P: Exploration, normalization, and summaries of high density oligonucleotide array probe level data. Biostatistics 2003, 4:249-264.
71. Dai M, Wang P, Boyd A D, Kostov G, Athey B, Jones E G, Bunney W E, Myers R M, Speed T P, Akil H, et al: Evolving gene/transcript definitions significantly alter the interpretation of GeneChip data. Nucleic Acids Res 2005, 33:e175.
72. Schaefer C F, Anthony K, Krupa S, Buchoff J, Day M, Hannay T, Buetow K H: PID: the Pathway Interaction Database. Nucleic Acids Res 2009, 37:D674-679.
73. Breitling R, Armengaud P, Amtmann A, Herzyk P: Rank products: a simple, yet powerful, new method to detect differentially regulated genes in replicated microarray experiments. FEBS Lett 2004, 573:83-92.
74. Symmans W F, Hatzis C, Sotiriou C, Andre F, Peintinger F, Regitnig P, Daxenbichler G, Desmedt C, Domont J, Marth C, et al: Genomic index of sensitivity to endocrine therapy for breast cancer. J Clin Oncol 2010, 28:4111-4119.
75. Greenman C, Stephens P, Smith R, Dalgliesh G L, Hunter C, Bignell G, Davies H, Teague J, Butler A, Stevens C, et al: Patterns of somatic mutation in human cancer genomes. Nature 2007, 446:153-158.
76. Venet D, Dumont J E, Detours V: Most random gene expression signatures are significantly associated with breast cancer outcome. PLoS computational biology 2011, 7:e1002240.
77. Starmans M H, Fung G, Steck H, Wouters B G, Lambin P: A simple but highly effective approach to evaluate the prognostic performance of gene expression signatures. PLoS One 2011, 6:e28320.
78. Boutros P C, Lau S K, Pintilie M, Liu N, Shepherd F A, Der S D, Tsao M S, Penn L Z, Jurisica I: Prognostic gene signatures for non-small-cell lung cancer. Proceedings of the National Academy of Sciences of the United States of America 2009, 106:2824-2828.
79. Hanahan D, Weinberg R A: Hallmarks of cancer: the next generation. Cell 2011, 144:646-674.
80. Matsushita H, Vesely M D, Koboldt D C, Rickert C G, Uppaluri R, Magrini V J, Arthur C D, White J M, Chen Y S, Shea L K, et al: Cancer exome analysis reveals a T-cell-dependent mechanism of cancer immunoediting. Nature 2012, 482:400-404.
81. Sorlie T, Perou C M, Tibshirani R, Aas T, Geisler S, Johnsen H, Hastie T, Eisen M B, van de Rijn M, Jeffrey S S, et al: Gene expression patterns of breast carcinomas distinguish tumor subclasses with clinical implications. Proceedings of the National Academy of Sciences of the United States of America 2001, 98:10869-10874.
82. Gangadhar T, Schilsky R L: Molecular markers to individualize adjuvant therapy for colon cancer. Nat Rev Clin Oncol 2010, 7:318-325.
83. Lau S K, Boutros P C, Pintilie M, Blackhall F H, Zhu C Q, Strumpf D, Johnston M R, Darling G, Keshavjee S, Waddell T K, et al: Three-gene prognostic classifier for early-stage non small-cell lung cancer. J Clin Oncol 2007, 25:5562-5569.
84. Kobel M, Kalloger S E, Boyd N, McKinney S, Mehl E, Palmer C, Leung S, Bowen N J, Ionescu D N, Rajput A, et al: Ovarian carcinoma subtypes are different diseases: implications for biomarker studies. PLoS Med 2008, 5:e232.
85. Curtis C, Shah S P, Chin S F, Turashvili G, Rueda O M, Dunning M J, Speed D, Lynch A G, Samarajiwa S, Yuan Y, et al: The genomic and transcriptomic architecture of 2,000 breast tumours reveals novel subgroups. Nature 2012, 486:346-352.
86. Perou C M: Molecular stratification of triple-negative breast cancers. Oncologist 2010, 15 Suppl 5:39-48.
87. Network TOGA: Comprehensive molecular portraits of human breast tumours. Nature 2012, 490:61-70.
88. Paik S, Shak S, Tang G, Kim C, Baker J, Cronin M, Baehner F L, Walker M G, Watson D, Park T, et al: A multigene assay to predict recurrence of tamoxifen-treated, node-negative breast cancer. N Engl J Med 2004, 351:2817-2826.
89. van't Veer L J, Dai H, van de Vijver M J, He Y D, Hart A A, Mao M, Peterse H L, van der Kooy K, Marton M J, Witteveen A T, et al: Gene expression profiling predicts clinical outcome of breast cancer. Nature 2002, 415:530-536.
90. Hudson T J, Anderson W, Artez A, Barker A D, Bell C, Bernabe R R, Bhan M K, Calvo F, Eerola I, Gerhard D S, et al: International network of cancer genome projects. Nature 2010, 464:993-998.
91. Wu G, Stein L: A network module-based method for identifying cancer prognostic signatures. Genome biology 2012, 13:R112.
92. Cerami E, Demir E, Schultz N, Taylor B S, Sander C: Automated network analysis identifies core pathways in glioblastoma. PLoS One 2010, 5:e8918.
93. Matthews L, Gopinath G, Gillespie M, Caudy M, Croft D, de Bono B, Garapati P, Hemish J, Hermjakob H, Jassal B, et al: Reactome knowledgebase of human biological pathways and processes. Nucleic Acids Res 2009, 37:D619-622.
94. Croft D, O'Kelly G, Wu G, Haw R, Gillespie M, Matthews L, Caudy M, Garapati P, Gopinath G, Jassal B, et al: Reactome: a database of reactions, pathways and biological processes. Nucleic Acids Res 2011, 39:D691-697.
95. Thiele I, Swainston N, Fleming R M, Hoppe A, Sahoo S, Aurich M K, Haraldsdottir H, Mo M L, Rolfsson O, Stobbe M D, et al: A community-driven global reconstruction of human metabolism. Nat Biotechnol 2013, 31:419-425.
96. Yoshihara K, Tsunoda T, Shigemizu D, Fujiwara H, Hatae M, Fujiwara H, Masuzaki H, Katabuchi H, Kawakami Y, Okamoto A, et al: High-risk ovarian cancer based on 126-gene expression signature is uniquely characterized by downregulation of antigen presentation pathway. Clin Cancer Res 2012, 18:1374-1385.
97. Navab R, Strumpf D, Bandarchi B, Zhu C Q, Pintilie M, Ramnarine V R, Ibrahimov E, Radulovich N, Leung L, Barczyk M, et al: Prognostic gene-expression signature of carcinoma-associated fibroblasts in non-small cell lung cancer. Proc Natl Acad Sci USA 2011, 108:7160-7165.
98. Marisa L, de Reynies A, Duval A, Selves J, Gaub M P, Vescovo L, Etienne-Grimaldi M C, Schiappa R, Guenot D, Ayadi M, et al: Gene expression classification of colon cancer into molecular subtypes: characterization, validation, and prognostic value. PLoS Med 2013, 10:e1001453.
99. Oh S C, Park Y Y, Park E S, Lim J Y, Kim S M, Kim S B, Kim J, Kim S C, Chu I S, Smith J J, et al: Prognostic gene expression signature associated with two molecularly distinct subtypes of colorectal cancer. Gut 2012, 61:1291-1298.
100. Smith J J, Deane N G, Wu F, Merchant N B, Zhang B, Jiang A, Lu P, Johnson J C, Schmidt C, Bailey C E, et al: Experimentally derived metastasis gene expression profile predicts recurrence and death in patients with colon cancer. Gastroenterology 2010, 138:958-968.
101. Chen H Y, Yu S L, Chen C H, Chang G C, Chen C Y, Yuan A, Cheng C L, Wang C H, Terng H J, Kao S F, et al: A five-gene signature and clinical outcome in non-small-cell lung cancer. The New England journal of medicine 2007, 356:11-20.
102. Lau S K, Boutros P C, Pintilie M, Blackhall F H, Zhu C Q, Strumpf D, Johnston M R, Darling G, Keshavjee S, Waddell T K, et al: Three-gene prognostic classifier for early-stage non small-cell lung cancer. Journal of clinical oncology: official journal of the American Society of Clinical Oncology 2007, 25:5562-5569.
103. Shedden K, Taylor J M, Enkemann S A, Tsao M S, Yeatman T J, Gerald W L, Eschrich S, Jurisica I, Giordano T J, Misek D E, et al: Gene expression-based survival prediction in lung adenocarcinoma: a multi-site, blinded validation study. Nature medicine 2008, 14:822-827.
104. Boutros P C, Lau S K, Pintilie M, Liu N, Shepherd F A, Der S D, Tsao M S, Penn L Z, Jurisica I: Prognostic gene signatures for non-small-cell lung cancer. Proceedings of the National Academy of Sciences of the United States of America 2009, 106:2824-2828.
105. Starmans M H, Pintilie M, John T, Der S D, Shepherd F A, Jurisica I, Lambin P, Tsao M S, Boutros P C: Exploiting the noise: improving biomarkers with ensembles of data analysis methodologies. Genome Med 2012, 4:84.
106. Yoshihara K, Tsunoda T, Shigemizu D, Fujiwara H, Hatae M, Masuzaki H, Katabuchi H, Kawakami Y, Okamoto A, Nogawa T, et al: High-risk ovarian cancer based on 126-gene expression signature is uniquely characterized by downregulation of antigen presentation pathway. Clinical cancer research: an official journal of the American Association for Cancer Research 2012, 18:1374-1385.
107. The Cancer Genome Atlas Research Network: Integrated genomic analyses of ovarian carcinoma. Nature 2011, 474:609-615.
108. Mankoo P K, Shen R, Schultz N, Levine D A, Sander C: Time to recurrence and survival in serous ovarian tumors predicted from integrated genomic profiles. PLoS One 2011, 6:e24709.
109. Wu G, Stein L: A network module-based method for identifying cancer prognostic signatures. Genome biology 2012, 13:R112.
110. Paik S, Shak S, Tang G, Kim C, Baker J, Cronin M, Baehner F L, Walker M G, Watson D, Park T, et al: A multigene assay to predict recurrence of tamoxifen-treated, node-negative breast cancer. N Engl J Med 2004, 351:2817-2826.
111. Haibe-Kains B, Schroeder B, Culhane A, Bontempi G, Sotiriou C, Quackenbush J: genefu R/Bioconductor package: Relevant Functions for Gene Expression Analysis, Especially in Breast Cancer. http://compbiodfciharvardedu 2011.
112. van't Veer L J, Dai H, van de Vijver M J, He Y D, Hart A A, Mao M, Peterse H L, van der Kooy K, Marton M J, Witteveen A T, et al: Gene expression profiling predicts clinical outcome of breast cancer. Nature 2002, 415:530-536.
113. The Cancer Genome Atlas Research Network: Comprehensive genomic characterization defines human glioblastoma genes and core pathways. Nature 2008, 455:1061-1068.
114. Bild A H, Yao G, Chang J T, Wang Q, Potti A, Chasse D, Joshi M B, Harpole D, Lancaster J M, Berchuck A, et al: Oncogenic pathway signatures in human cancers as a guide to targeted therapies. Nature 2006, 439:353-357.
115. Chin K, DeVries S, Fridlyand J, Spellman P T, Roydasgupta R, Kuo W L, Lapuk A, Neve R M, Qian Z, Ryder T, et al: Genomic and transcriptional aberrations linked to breast cancer pathophysiologies. Cancer Cell 2006, 10:529-541. 116. Desmedt C, Piette F, Loi S, Wang Y, Lallemand F, Haibe-Kains B, Viale G, Delorenzi M, Zhang Y, d'Assignies M S, et al: Strong time dependence of the 76-gene prognostic signature for node-negative breast cancer patients in the TRANSBIG multicenter independent validation series. Clin Cancer Res 2007, 13:3207-3214.
117. Li Y, Zou L H, Li Q Y, Haibe-Kains B, Tian R Y, Li Y, Desmedt C, Sotiriou C, Szallasi Z, Iglehart J D, et al: Amplification of LAPTM4B and YWHAZ contributes to chemotherapy resistance and recurrence of breast cancer. Nature Medicine 2010, 16:214-U121.
118. Loi S, Haibe-Kains B, Desmedt C, Wirapati P, Lallemand F, Tutt A M, Gillet C, Ellis P, Ryder K, Reid J F, et al: Predicting prognosis using molecular profiling in estrogen receptor-positive breast cancer treated with tamoxifen. BMC Genomics 2008, 9:239.
119. Miller L D, Smeds J, George J, Vega V B, Vergara L, Ploner A, Pawitan Y, Hall P, Klaar S, Liu E T, Bergh J: An expression signature for p53 status in human breast cancer predicts mutation status, transcriptional effects, and patient survival. Proc Natl Acad Sci USA 2005, 102:13550-13555.
120. Pawitan Y, Bjohle J, Amler L, Borg A L, Egyhazi S, Hall P, Han X, Holmberg L, Huang F, Klaar S, et al: Gene expression profiling spares early breast cancer patients from adjuvant therapy: derived and validated in two population-based cohorts. Breast Cancer Res 2005, 7:R953-964.
121. Sabatier R, Finetti P, Cervera N, Lambaudie E, Esterni B, Mamessier E, Tallet A, Chabannon C, Extra J M, Jacquemier J, et al: A gene expression signature identifies two prognostic subgroups of basal breast cancer. Breast Cancer Res Treat 2010.
122. Schmidt M, Bohm D, von Torne C, Steiner E, Puhl A, Pilch H, Lehr H A, Hengstler J G, Kolbl H, Gehrmann M: The humoral immune system has a key prognostic impact in node-negative breast cancer. Cancer Research 2008, 68:5405-5413.
123. Sotiriou C, Wirapati P, Loi S, Harris A, Fox S, Smeds J, Nordgren H, Farmer P, Praz V, Haibe-Kains B, et al: Gene expression profiling in breast cancer: understanding the molecular basis of histologic grade to improve prognosis. J Natl Cancer Inst 2006, 98:262-272.
124. Symmans W F, Hatzis C, Sotiriou C, Andre F, Peintinger F, Regitnig P, Daxenbichler G, Desmedt C, Domont J, Marth C, et al: Genomic index of sensitivity to endocrine therapy for breast cancer. J Clin Oncol 2010, 28:4111-4119.
125. Wang Y, Klijn J G, Zhang Y, Sieuwerts A M, Look M P, Yang F, Talantov D, Timmermans M, Meijer-van Gelder M E, Yu J, et al: Gene-expression profiles to predict distant metastasis of lymph-node-negative primary breast cancer. Lancet 2005, 365:671-679.
126. Zhang Y, Sieuwerts A, McGreevy M, Graham C, Cufer T, Paradiso A, Harbeck N, Span P N, Hicks D G, Crowe J, et al: The 76-Gene Signature Defines High-Risk Patients That Benefit from Adjuvant Tamoxifen Therapy. Cancer Research 2009, 69:598S-599S.
127. Jorissen R N, Gibbs P, Christie M, Prakash S, Lipton L, Desai J, Kerr D, Aaltonen L A, Arango D, Kruhoffer M, et al: Metastasis-Associated Gene Expression Changes Predict Poor Outcomes in Patients with Dukes Stage B and C Colorectal Cancer. Clinical cancer research: an official journal of the American Association for Cancer Research 2009, 15:7642-7651.
128. Loboda A, Nebozhyn M V, Watters J W, Buser C A, Shaw P M, Huang P S, Van′t Veer L, Tollenaar R A, Jackson D B, Agrawal D, et al: EMT is the dominant program in human colon cancer. BMC medical genomics 2011, 4:9.
129. The Cancer Genome Atlas Research Network: Comprehensive molecular characterization of human colon and rectal cancer. Nature 2012, 487:330-337.
130. Beer D G, Kardia S L, Huang C C, Giordano T J, Levin A M, Misek D E, Lin L, Chen G, Gharib T G, Thomas D G, et al: Gene-expression profiles predict survival of patients with lung adenocarcinoma. Nature medicine 2002, 8:816-824.
131. Bhattacharjee A, Richards W G, Staunton J, Li C, Monti S, Vasa P, Ladd C, Beheshti J, Bueno R, Gillette M, et al: Classification of human lung carcinomas by mRNA expression profiling reveals distinct adenocarcinoma subclasses. Proc Natl Acad Sci USA 2001, 98:13790-13795.
132. Lu Y, Lemon W, Liu P Y, Yi Y, Morrison C, Yang P, Sun Z, Szoke J, Gerald W L, Watson M, et al: A gene expression signature predicts survival of patients with stage I non-small cell lung cancer. PLoS Med 2006, 3:e467.
133. Zhu C Q, Ding K, Strumpf D, Weir B A, Meyerson M, Pennell N, Thomas R K, Naoki K, Ladd-Acosta C, Liu N, et al: Prognostic and predictive gene signature for adjuvant chemotherapy in resected non-small-cell lung cancer. Journal of clinical oncology: official journal of the American Society of Clinical Oncology 2010, 28:4417-4424.
134. Bonome T, Levine D A, Shih J, Randonovich M, Pise-Masison C A, Bogomolniy F, Ozbun L, Brady J, Barrett J C, Boyd J, Birrer M J: A gene signature predicting for survival in suboptimally debulked patients with ovarian cancer. Cancer Res 2008, 68:5478-5486.
135. Denkert C, Budczies J, Darb-Esfahani S, Gyorffy B, Sehouli J, Konsgen D, Zeillinger R, Weichert W, Noske A, Buckendahl A C, et al: A prognostic gene expression index in ovarian cancer—validation across different independent data sets. J Pathol 2009, 218:273-280.
136. Konstantinopoulos P A, Spentzos D, Karlan B Y, Taniguchi T, Fountzilas E, Francoeur N, Levine D A, Cannistra S A: Gene expression profile of BRCAness that correlates with responsiveness to chemotherapy and with outcome in patients with epithelial ovarian cancer. Journal of clinical oncology: official journal of the American Society of Clinical Oncology 2010, 28:3555-3561.
137. Tothill R W, Tinker A V, George J, Brown R, Fox S B, Lade S, Johnson D S, Trivett M K, Etemadmoghadam D, Locandro B, et al: Novel molecular subtypes of serous and endometrioid ovarian cancer linked to clinical outcome. Clin Cancer Res 2008, 14:5198-5208.

1.-22. (canceled)

23. A method of prognosing or classifying a patient comprising:
determining mRNA abundance using a sample of a breast cancer tumour of the patient for the group of genes comprising: GSK3B, AKT1S1, RHEB, TSC1, TSC2, RPS6KB1, RPTOR, MTOR, RICTOR, ERBB2, MKI67, ESR1 and PGR, each of said genes associated with at least one node of the PIK3 cell signalling pathway;
constructing an expression profile from the mRNA abundance;
comparing said expression profile to a plurality of reference expression profiles and comparing clinical indicators of the patient to a plurality of reference clinical indicators, wherein the clinical indicators comprise N-stage and tumour size, and wherein each of the plurality of reference expression profiles and each of the reference clinical indicators are associated with a predetermined residual risk of breast cancer; and
selecting the reference expression profile most similar to the expression profile and the reference clinical indicators most similar to the patient clinical indicators, to obtain a residual risk associated with breast cancer.

24. The method of claim 23, wherein the genes further comprise EGFR, ERBB3, and ERBB4.

25. The method of claim 23, wherein the residual risk is expressed as distant metastasis free survival.

26. The method of claim 25, wherein the residual risk is expressed as either low or high risk of breast cancer occurrence.

27. The method of claim 23, further comprising normalizing said mRNA abundance using at least one control.

28. The method of claim 27, wherein said at least one control comprises a plurality of controls.

29. The method of claim 28, wherein at least one of the plurality of controls comprises mRNA abundance of reference genes of a reference patient.

30. The method of claim 28, wherein at least one of the plurality of controls comprises mRNA abundance of reference genes of the patient.

31. The method of claim 23, wherein comparing said expression profile to the plurality of reference expression profiles further comprises:
a) determining dysregulation of each of the at least one nodes by calculating a score proportional to a degree of dysregulation in each of the at least one nodes from said normalized mRNA abundance; and
b) wherein selecting the reference expression profile and the reference clinical indicators further comprises:
i) inputting the dysregulation score into a model trained with a plurality of reference scores and plurality of reference clinical indicators; and
ii) inputting clinical indicators of the patient into the model.

32. The method of claim 23, wherein determining mRNA abundance comprises use of quantitative PCR.

33.-54. (canceled)

55. A computer-implemented method of prognosing or classifying a patient, the method comprising:
a) receiving, at least one processor, data reflecting mRNA abundance determined using a sample of a breast cancer tumour of the patient for the group of genes comprising: GSK3B, AKT1S1, RHEB, TSC1, TSC2, RPS6KB1, RPTOR, MTOR, RICTOR, ERBB2, MKI67, ESR1 and PGR, each of said genes associated with at least one node of the PIK3 cell signalling pathway;
b) constructing, at the at least one processor, an expression profile from the data reflecting mRNA abundance;
c) comparing, at the at least one processor, said expression profile to a plurality of reference expression profiles and comparing clinical indicators of the patient to a plurality of reference clinical indicators, wherein the clinical indicators comprise N-stage and tumour size, and wherein each of the plurality of reference expression profiles and each of the reference clinical indicators are associated with a predetermined residual risk of breast cancer; and
d) selecting, at the at least one processor, the reference expression profile most similar to the expression profile and the reference clinical indicators most similar to the patient clinical indicators, to obtain a residual risk associated with breast cancer.

56. The method of claim 55, wherein the genes further comprise EGFR, ERBB3, and ERBB4.

57. The method of claim 55, wherein the residual risk is expressed as distant metastasis free survival.

58. The method of claim 57, wherein the residual risk is expressed as either low or high risk of breast cancer occurrence.

59. The method of claim 55, further comprising normalizing, at the at least one processor, said mRNA abundance using at least one control.

60. The method of claim 59, wherein said at least one control comprises a plurality of controls.

61. The method of claim 60, wherein at least one of the plurality of controls comprises mRNA abundance of reference genes of a reference patient.

62. The method of claim 60, wherein at least one of the plurality of controls comprises mRNA abundance of reference genes of the patient.

63. The method of claim 55, wherein comparing said expression profile to the plurality of reference expression profiles further comprises:
determining, at the at least one processor, dysregulation of each of the at least one nodes by calculating a score proportional to a degree of dysregulation in each of the at least one nodes from said mRNA abundance; and
wherein selecting the reference expression profile and the reference clinical indicators further comprises:
inputting the dysregulation score into a model trained with a plurality of reference scores and plurality of reference clinical indicators; and
inputting clinical indicators of the patient into the model.

64.-84. (canceled)

85. A device for prognosing or classifying a patient, the device comprising:
at least one processor; and
electronic memory in communication with the at one processor, the electronic memory storing processor-executable code that, when executed at the at least one processor, causes the at least one processor to:
a) receive data reflecting mRNA abundance determined using a sample of a breast cancer tumour of the patient for the group of genes comprising: GSK3B, AKT1S1, RHEB, TSC1, TSC2, RPS6KB1, RPTOR, MTOR, RICTOR, ERBB2, MKI67, ESR1 and PGR, each of said genes associated with at least one node of the PIK3 cell signalling pathway;
b) construct an expression profile from the data reflecting mRNA abundance;
c) compare said expression profile to a plurality of reference expression profiles and comparing clinical indicators of the patient to a plurality of reference clinical indicators, wherein the clinical indicators comprise N-stage and tumour size, and wherein each of the plurality of reference expression profiles and each of the reference clinical indicators are associated with a predetermined residual risk of breast cancer; and
d) select the reference expression profile most similar to the expression profile and the reference clinical indicators most similar to the patient clinical indicators, to obtain a residual risk associated with breast cancer.

86.-93. (canceled)

94. A method of treating a patient, comprising:
a) determining the disease relapse risk of the patient according to the method of claim 1; and
b) selecting a treatment based on the disease relapse risk, and preferably treating the patient according to the treatment.

95. An array comprising one or more polynucleotide probes complementary and hybridizable to an expression product of each of a plurality of genes comprising GSK3B, AKT1S1, RHEB, TSC1, TSC2, RPS6KB1, RPTOR, MTOR, RICTOR, ERBB2, MKI67, ESR1 and PGR.

96. The array of claim 95, wherein the plurality of genes further comprises EGFR, ERBB3, ERBB4.

97.-125. (canceled)
